(19) United States
(12) Patent Application Publication
(10) Pub. No.: US 2013/0129718 A1
(43) Pub. Date: May 23, 2013

(54) PURIFICATION OF ANTI-C-MET ANTIBODIES

(71) Applicant: Genentech, Inc., South San Francisco, CA (US)

(72) Inventors: Marc Wong, San Carlos, CA (US); Jerome Joseph Bill, JR., San Mateo, CA (US); Arick Michael Brown, Pacifica, CA (US); Glen Scott Giese, Belmont, CA (US); Judy Fay-Chen Hsii, Redwood City, CA (US); Amy Lim, Palo Alto, CA (US); Josefine Persson, Half Moon Bay, CA (US); Asha Nandini Radhamohan, Mountain View, CA (US); Maricel Rodriguez, South San Francisco, CA (US)

(73) Assignee: GENENTECH, INC., South San Francisco, CA (US)

(21) Appl. No.: 13/681,980

(22) Filed: Nov. 20, 2012

Related U.S. Application Data
(60) Provisional application No. 61/562,429, filed on Nov. 21, 2011.
(60) Provisional application No. 61/562,925, filed on Nov. 22, 2011.

(51) Int. Cl. C07K 1/14; C07K 1/22; A61K 45/06; C07K 1/34; A61K 39/395; C07K 1/18; C07K 16/32; C07K 1/36

(52) U.S. Cl. CPC C07K 1/14; C07K 16/32; C07K 1/22; C07K 1/36; C07K 1/34; A61K 39/39558; C07K 1/18; A61K 45/06
USPC 424/133.1; 530/387.7; 530/387.3; 424/138.1; 435/375

(57) ABSTRACT

Provided herein are methods of purifying anti-c-met antibodies, compositions and pharmaceutical formulations comprising purified anti-c-met antibodies, and methods of using the same.

### RELATED APPLICATIONS

This application claims priority under 35 USC 119(e) to U.S. provisional patent application No. 61/562,429 filed Nov. 21, 2011 and U.S. provisional patent application No. 61/562,925 filed Nov. 22, 2011, the contents of which are incorporated herein by reference in their entirety.

### SEQUENCE LISTING

This application contains a Sequence Listing submitted via EFS-Web and hereby incorporated by reference in its entirety. Said ASCII copy, created on Nov. 16, 2012, is named Sequence Listing, and is 22,046 bytes in size

### TECHNICAL FIELD

Provided herein are methods of purifying anti-c-met antibodies, compositions and pharmaceutical formulations comprising purified anti-c-met antibodies, and methods of using the same.

### BACKGROUND

Biologics such as therapeutic antibodies are produced from recombinant systems, which comprise complex concentrated mixtures of components, and can therefore be contaminated with components of the host cell system used to manufacture the therapeutic antibody. Frequently, even after multiple purification steps, significant levels of those contaminants may be present. Patient safety necessitates that the contaminants be eliminated or reduced to the lowest levels practical to prevent safety and efficacy problems. Failure to identify and sufficiently remove contaminates can result in reduced drug efficacy or adverse patient reactions such as adverse immune reactions. For example, the outer membrane of Escherichia coli (E. coli) comprises lipopolysacchrides (LPS), which can act as an endotoxin and elicit a strong immune response, high fever, if not removed. The removal of contaminants can have significant cost implications in drug development and manufacture processes.

For E. coli cultured therapeutic antibodies, the contaminants can be components of the growth media and/or host cells used for propagation, DNA or RNA vectors, E. coli proteins (ECP), lipids, and/or LPS. In addition to potentially directly effecting drug efficacy and/or safety, a number of contaminants, including ECP, phospholipids, endotoxins, and DNA/RNA, (including vector sequences), can form complexes with the therapeutic antibody as a result of hydrophobic interactions, metal bridging, and/or charge complexation, which can lead to aggregation of the therapeutic antibody. Further, therapeutic antibodies produced in E. coli accumulate internally in the periplasm, and the cells need to be ruptured to isolate the therapeutic antibody. Host protease activity commonly occurs during the cell disruption and can substantially decrease yield and result in proteolysis of the therapeutic antibody without efficient purification. Multiple rounds of chromatography and purification steps are required to separate the growth media and/or host cell contaminants from the therapeutic antibody.

In addition to the growth media and/or host cell contaminants, the recovery and purification process itself can introduce contaminants depending on the type of adsorbent utilized in the chromatography method. For example, during protein A affinity chromatography, protein A ligand can co-elute with the therapeutic antibody. Further, in the case of protein A, there is some evidence that suggests that protein A may cause adverse physiological events. M. Gomez et al. Nat. Med. 10:842 (2004). The processes of removing contaminants can be extensive, and every step of recovery and purification also results in significant loss of yield and potential introduction of further contaminants.

Despite the importance of removing contaminates, there is no universal purification scheme which will be effective for all polypeptides. Polypeptide properties such as the molecular weight, isoelectric point (pI), hydrophobicity, protease sensitivity, charge properties and distribution, post-translation modifications, and/or solubility vary significantly among polypeptide. These properties can significantly influence the purification scheme and ability to remove contaminants.

Numerous molecules targeted at the HGF/c-met pathway have been reported. These molecules include a portion of the extracellular domain of c-met and anti-c-met antibodies such as those described in U.S. Pat. No. 5,686,292, Martens, T. et al., Clin. Cancer Res. 12 (20 Pt. 1):6144 (2006), U.S. Pat. No. 6,468,529, WO2006/015371, WO2007/063816, and WO2010/045345. Bivalent forms of anti-c-met antibodies have been shown to promote dimerization and lead to activation of c-met (agonistic function), while conversely monovalent antibodies have been shown to inhibit c-met activity (antagonistic function). For treatment of pathological conditions requiring an antagonistic function, bivalency of an anti-c-met antibody could result in an undesirable agonistic effect, and therefore, the monovalent trait is required to ensure an antagonistic activity upon binding of the anti-c-met antibody to the target for treatment of the pathological condition. Fab fragments and one-armed antibodies are examples of monovalent antibodies. One-armed antibodies generally have a longer half-life than Fabs. However, a concern in utilizing a one-armed antibody, which comprises a single light chain and a single heavy chain (as well as an additional Fc region), is the potential failure to maintain the one-armed antibody structure. Aggregation of monovalent antibodies (formation of multimer and oligomers) and/or failure to maintain monovalent structure, rather than a bivalent antibody with two heavy chain and two light chains, during production and purification could lead to an undesirable agonistic effect. Minimization of anti-c-met antibody aggregation and stabilization of the monovalent structure during purification and in the purified product is thus particularly important.

Onartuzumab is an anti-c-met antibody and is the first one-armed antibody to be produced in E. coli. The purification process of onartuzumab is further complicated by the very similar electrostatic properties of onartuzumab and host cell impurities/contaminants since many conventional methods of antibody purification rely on differences in electrostatic properties between the antibody and host cell impurity/contaminant to facilitate separation. Therefore, despite the significant advancements in production and purifications of biologics generally and the development of molecules which target the HGF/c-met pathway, efficient purification methods which minimize contaminants and impurities while retaining antagonistic activity of anti-c-met antibodies, particularly in the one-armed format, are still needed

All references cited herein, including patent applications and publications, are incorporated by reference in their entirety.

### SUMMARY

Provided herein are methods of purifying an anti-c-met antibody and compositions comprising purified anti-c-met antibodies. Provided herein are compositions comprising an anti-c-met antibody, wherein host cell protein (HCP) is present in less than or equal to about 50 ng/mg. Further provided herein are lots (e.g., batches) of compositions comprising an anti-c-met antibody, wherein HCP is present in less than or equal to about 50 ng/mg.

Provided herein are methods of purifying an anti-c-met antibody comprising keeping a composition comprising the anti-c-met antibody at a temperature of greater than 28° C. and a pH between about pH 6 and about pH 8 for more than 6 hours. In some embodiments, the method further comprises centrifuging the composition comprising the anti-c-met antibody. In some embodiments, the method further comprises loading the composition comprising the anti-c-met antibody on protein A resin comprising an agarose matrix (e.g., MabSelect SuRe™ resin) and eluting the anti-c-met antibody.

Provided herein are methods of purifying an anti-c-met antibody comprising loading a composition comprising an anti-c-met antibody on protein A resin comprising an agarose matrix (e.g., MabSelect SuRe™ resin) and eluting the anti-c-met antibody. In some embodiments, the method further comprises loading the composition comprising the anti-c-met antibody on a weak anion exchange resin and recovering the anti-c-met antibody in the flow-through. In some embodiments, the weak anion exchange resin is run in flow-through mode.

Provided herein are methods of purifying an anti-c-met antibody comprising loading a composition comprising an anti-c-met antibody on a weak anion exchange resin and recovering the anti-c-met antibody in the flow-through. In some embodiments, the weak anion exchange resin is run in flow-through mode.

In some embodiments of any of the methods of purification, the method further comprises loading the composition comprising the anti-c-met antibody on a strong cation exchange resin and eluting the anti-c-met antibody.

In some embodiments of any of the methods of purification, the method further comprises loading the composition comprising the anti-c-met antibody on a strong anion exchange resin and eluting the anti-c-met antibody.

In some embodiments of any of the methods of purification, the method further comprises ultrafiltering and/or diafiltering the composition comprising the anti-c-met antibody.

Further provided herein are compositions comprising an anti-c-met antibody purified or obtainable by any of the methods of purification described above. In addition provided herein are lots (e.g., batches) of compositions comprising an anti-c-met antibody purified or obtainable by any of the methods of purification described above.

Provided are also pharmaceutical formulations comprising a composition or lot of any of the compositions described above. In some embodiments, the pharmaceutical formulations are liquid pharmaceutical formulations. In some embodiments, the pharmaceutical formulations are suitable for administration to an individual (e.g., human).

In some embodiments of any of the methods of purifying, compositions, and/or pharmaceutical formulations, the HCP in the composition comprising an anti-c-met antibody is less than or equal to about 50 ng/mg. In some embodiments of any of the methods of purifying, compositions, and/or pharmaceutical formulations, the average HCP in a lot (e.g., batch) of the composition comprising an anti-c-met antibody is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is E. coli cell protein (e.g., ECP) and/or average ECP.

In some embodiments of any of the methods of purifying, compositions, and/or pharmaceutical formulations, the DNA levels in the composition comprising an anti-c-met antibody are less than or equal to about 0.3 pg/mg. In some embodiments of any of the methods of purifying, compositions, and/or pharmaceutical formulations, the average DNA levels in a lot (e.g., batch) of the composition comprising an anti-c-met antibody are less than or equal to about 0.3 pg/mg. In some embodiments, the DNA levels and/or average DNA levels are less than or equal to about any of 0.3 pg/mg, 0.25 pg/mg, 0.2 pg/mg, 0.15 pg/mg, or 0.1 pg/mg. In some embodiments, the DNA levels and/or average DNA levels are between about any of 0.001 pg/mg and 0.3 pg/mg, 0.001 pg/mg and 0.2 pg/mg, 0.001 pg/mg and 0.1 pg/mg, 0.01 pg/mg and 0.3 pg/mg, 0.01 pg/mg and 0.2 pg/mg, or 0.01 pg/mg and 0.1 pg/mg. In some embodiments, the DNA levels and/or average DNA levels are about any of 0.3, 0.25, 0.2, 0.15, or 0.1 pg/mg.

In some embodiments of any of the methods of purifying, compositions, and/or pharmaceutical formulations, the leached protein A (i.e., LpA) in the composition comprising an anti-c-met antibody is less than or equal to about 2 ng/mg. In some embodiments of any of the methods of purifying, compositions, and/or pharmaceutical formulations, the average LpA in a lot (e.g., batch) of the composition comprising an anti-c-met antibody is less than or equal to about 2 ng/mg. In some embodiments, the LpA and/or average LpA is between about any of 0.001 ng/mg and 2 ng/mg, 0.01 ng/mg and 2 ng/mg, 0.1 ng/mg and 2 ng/mg, or 1 ng/mg and 2 ng/mg. In some embodiments, the LpA and/or average LpA is about any of 1, 1.25, 1.5, 1.75, or 2 ng/mg.

In some embodiments of any of the methods of purifying, compositions, and/or pharmaceutical formulations, the Limulus Amebocyte Lysate (i.e., LAL) in the composition comprising an anti-c-met antibody is less than or equal to about 0.01 EU/mg. In some embodiments of any of the methods of purifying, compositions, and/or pharmaceutical formulations, the average LAL in a lot (e.g., batch) of the composition comprising an anti-c-met antibody is less than or equal to about 0.01 EU/mg. In some embodiments, the LAL and/or average LAL is less than or equal to about any of 0.007 EU/mg, 0.006 EU/mg, 0.005 EU/mg, 0.002 EU/mg, or 0.001 EU/mg. In some embodiments, the LAL and/or average LAL is between about any of 0.0001 EU/mg and 0.01 EU/mg, 0.0001 EU/mg and 0.007 EU/mg, 0.0001 EU/mg and 0.006 EU/mg, or 0.0001 EU/mg and 0.005 EU/mg. In some embodiments, the LAL and/or average LAL is about any of 0.01, 0.007, 0.006, 0.005, 0.004, 0.003, or 0.002 EU/mg.

In some embodiments of any of the methods of purifying, compositions, and/or pharmaceutical formulations, the percentage of aggregates in the composition comprising an anti-c-met antibody is less than or equal to about 0.3%. In some embodiments of any of the methods of purifying, compositions, and/or pharmaceutical formulations, the average percentage of aggregates in a lot (e.g., batch) of the composition comprising an anti-c-met antibody is less than or equal to about 0.3%. In some embodiments, the percentage of aggregates and/or average percentage of aggregates is less than or equal to about any of 0.2% or 0.1%. In some embodiments, the percentage of aggregates and/or average percentage of aggregates is between about any of 0.001% and 0.3%, 0.01% and 0.3%, 0.001% and 0.2%, or 0.01% and 0.2%. In some embodiments, the percentage of aggregates and/or average percentage of aggregates is about any of 0.3%, 0.25%, 0.2%, 0.15%, or 0.1%.

In some embodiments of any of the methods of purifying, compositions, and/or pharmaceutical formulations, the percentage of monomer in the composition comprising an anti-c-met antibody is greater than or equal to about 99.5%. In some embodiments of any of the methods of purifying, compositions, and/or pharmaceutical formulations, the average percentage monomer in a lot (e.g., batch) of the composition comprising an anti-c-met antibody is greater than or equal to about 99.5%. In some embodiments, the percentage of monomer and/or average percentage of monomer is greater than or equal to about any of 99.6%, 99.7%, 99.8%, or 99.9%. In some embodiments, the percentage of monomer and/or average percentage of monomer is between about any of 99.5% and 99.999%, 99.5% and 99.99%, 99.6% and 99.999%, 99.6% and 99.99%, 99.7% and 99.999%, 99.7% and 99.99%, 99.8% and 99.999%, 99.8% and 99.99%, or 99.9% and 99.999%, 99.9% and 99.99%. In some embodiments, the percentage of monomer and/or average percentage of monomer is about any of 99.5%, 99.6%, 99.7%, 99.8%, or 99.9%.

In some embodiments of any of the methods of purifying, compositions, and/or pharmaceutical formulations, the percentage of fragments in the composition comprising an anti-c-met antibody is less than or equal to about 0.3%. In some embodiments of any of the methods of purifying, compositions, and/or pharmaceutical formulations, the average percentage of fragments in a lot (e.g., batch) of the composition comprising an anti-c-met antibody is less than or equal to about 0.3%. In some embodiments, the percentage of fragments and/or average percentage of fragments is less than or equal to about any of 0.2% or 0.1%. In some embodiments, the percentage of fragments and/or average percentage of fragments is between about any of 0.001% and 0.3%, 0.01% and 0.3%, 0.001% and 0.2%, or 0.01% and 0.2%. In some embodiments, the percentage of fragments and/or average percentage of fragments is about any of 0.3%, 0.25%, 0.2%, 0.15%, 0.1%, or 0%. In some embodiments, fragments are not detectable.

In some embodiments of any of the methods of purifying, compositions, and/or pharmaceutical formulations, the percentage of acidic variants in the composition comprising an anti-c-met antibody is less than or equal to about 20%. In some embodiments of any of the methods of purifying, compositions, and/or pharmaceutical formulations, the average percentage of acidic variants in a lot (e.g., batch) of the composition comprising an anti-c-met antibody is less than or equal to about 20%. In some embodiments, the percentage of acidic variants and/or average percentage of acidic variants is less than or equal to about any of 20%, 18.5%, 17.5%, 15%, 12.5%. In some embodiments, the percentage of acidic variants and/or average percentage of acidic variants is between about any of 1% and 20%, 5% and 20%, or 10% and 20%. In some embodiments, the percentage of acidic variants and/or average percentage of acidic variants is about any of 20%, 18.5%, 17.5%, 15%, or 12.5%.

In some embodiments of any of the methods of purifying, compositions, and/or pharmaceutical formulations, the percentage of main peak in the composition comprising an anti-c-met antibody is greater than or equal to about 75%. In some embodiments of any of the methods of purifying, compositions, and/or pharmaceutical formulations, the average percentage of main peak in a lot (e.g., batch) of the composition comprising an anti-c-met antibody is greater than or equal to about 75%. In some embodiments, the percentage of main peak and/or average percentage of main peak greater than or equal to about any of 77.5%, 80%, 82.5%, or 85%. In some embodiments, the percentage of main peak and/or average percentage of main peak is between about any of 75% and 95%, 77.5% and 95%, 80% and 95%, 82.5% and 95%, or 85% and 95%. In some embodiments, the percentage of main peak and/or average percentage of main peak is about any of 75%, 77.5%, 80%, 82.5%, or 85%.

In some embodiments of any of the methods of purifying, compositions, and/or pharmaceutical formulations, the percentage of basic variants in the composition comprising an anti-c-met antibody is less than or equal to about 2.0%. In some embodiments of any of the methods of purifying, compositions, and/or pharmaceutical formulations, the average percentage of basic variants in a lot (e.g., batch) of the composition comprising an anti-c-met antibody is less than or equal to about 2.0%. In some embodiments, the percentage of basic variants and/or average percentage of basic variants is less than or equal to about any of 1.5%, 1.25%, 1.1%, or 1%. In some embodiments, the percentage of basic variants and/or average percentage of basic variants is between about any of 0.001% and 2%, 0.01% and 2%, 0.001% and 1.5%, or 0.01% and 1.5%, 0.001% and 1.0%, or 0.01% and 1.0%. In some embodiments, the percentage of basic variants and/or average percentage of basic variants is about any of 2%, 1.5%, 1.25%, 1.1%, or 1%.

For example, provided are compositions and/or lots (e.g., batches) comprising a composition comprising an anti-c-met antibody, wherein HCP is present in less than or equal to about 50 ng/mg, the DNA levels in the composition comprising an anti-c-met antibody are less than or equal to about 0.3 pg/mg, the LpA in the composition comprising an anti-c-met antibody is less than or equal to about 2 ng/mg, the Limulus Amebocyte Lysate (LAL) in the composition comprising an anti-c-met antibody is less than or equal to about 0.01 EU/mg, the percentage of aggregates in the composition comprising an anti-c-met antibody is less than or equal to about 0.3%, the percentage of monomer in the composition comprising an anti-c-met antibody is greater than or equal to about 99.5%, the percentage of fragments in the composition comprising an anti-c-met antibody is less than or equal to about 0.3%, the percentage of acidic variants in the composition comprising an anti-c-met antibody is less than or equal to about 20%, the percentage of main peak in the composition comprising an anti-c-met antibody is greater than or equal to about 75%, and the percentage of basic variants in the composition comprising an anti-c-met antibody is less than or equal to about 2.0%. In addition, provided herein are composition and/or lots (e.g., batches) comprising a composition comprising an anti-c-met antibody, wherein HCP is present in less than or equal to about 15 ng/mg, the DNA levels in the composition comprising an anti-c-met antibody are less than or equal to about 0.3 pg/mg, the LpA in the composition comprising an anti-c-met antibody is less than or equal to about 2 ng/mg, the Limulus Amebocyte Lysate (LAL) in the composition comprising an anti-c-met antibody is less than or equal to about 0.01 EU/mg, the percentage of aggregates in the composition comprising an anti-c-met antibody is less than or equal to about 0.3%, the percentage of monomer in the composition comprising an anti-c-met antibody is greater than or equal to about 99.5%, the percentage of fragments in the composition comprising an anti-c-met antibody is less than or equal to about 0.3%, the percentage of acidic variants in the composition comprising an anti-c-met antibody is less than or equal to about 20%, the percentage of main peak in the composition comprising an anti-c-met antibody is greater than or equal to about 75%, and the percentage of basic variants in the composition comprising an anti-c-met antibody is less than or equal to about 2.0%.

In some embodiments of any of the methods of purifying, compositions, and/or pharmaceutical formulations, the anti-c-met antibody is an antibody described in Section IV. In some embodiments of any of the methods of purifying, compositions, and/or pharmaceutical formulations, the anti-c-met antibody is about 100 kDa. In some embodiments of any of the methods of purifying, compositions, and/or pharmaceutical formulations, the anti-c-met antibody has a pI of about 8.2, about 8.3, and/or about 8.4. In some embodiments of any of the methods of purifying, compositions, and/or pharmaceutical formulations, the anti-c-met antibody comprises a single antigen binding arm capable of binding to c-met. In some embodiments of any of the methods of purifying, compositions, and/or pharmaceutical formulations, the anti-c-met antibody is monovalent. In some embodiments of any of the methods of purifying, compositions, and/or pharmaceutical formulations, the anti-c-met antibody is onartuzumab.

In some embodiments of any of the methods of purifying, compositions, and/or pharmaceutical formulations, the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6). In some embodiments, the anti-c-met antibody comprises (a) a heavy chain variable domain comprising the sequence: EVQLVESGGGLVQPGGSLRLSCAASGYTFTSYWLHWVRQAPGKGLEWVGMIDPSNSDTRFNPN FKDRFTISADTSKNTAYLQMNSLRAEDTAVYYCATYRSYVTPLDYWGQGTLVTVSS (SEQ ID NO:19) and (b) a light chain variable domain comprising the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:20). In some embodiments, the anti-c-met antibody is monovalent. In some embodiments, the anti-c-met antibody is an anti-c-met antibody fragment. In some embodiments, the anti-c-met antibody is a one-armed antibody. In some embodiments, the anti-c-met antibody comprises a single antigen binding arm and comprises a Fc region, wherein the Fc region comprises a first and a second Fc polypeptide, and wherein the first and second Fc polypeptides are present in a complex. In some embodiments, the first and second Fc polypeptides form a Fc region that increases stability of said antibody fragment compared to a Fab molecule comprising said antigen binding arm. In some embodiments, the anti-c-met antibody comprises (a) a first polypeptide comprising the amino acid sequence of SEQ ID NO:19, a CH1 sequence, and a first Fc polypeptide and (b) a second polypeptide comprising the amino acid sequence of SEQ ID NO:20 and CL1 sequence. In some embodiments, the anti-c-met antibody further comprises (c) a third polypeptide comprising a second Fc polypeptide. In some embodiments, the first Fc polypeptide comprises the Fc sequence depicted in FIG. 1 (SEQ ID NO: 17) and the second Fc polypeptide comprises the Fc sequence depicted in FIG. 2 (SEQ ID NO: 18). In some embodiments, the anti-c-met antibody is onartuzumab. In some embodiment, anti-c-met antibody binds the same epitope as onartuzumab.

Further provided herein are methods of inhibiting c-met activated cell proliferation, said method comprising contacting a cell or tissue with an effective amount of a composition, lot, and/or pharmaceutical formulation described above.

Provided herein are methods of modulating a disease associated with dysregulation of the HGF/c-met signaling axis, said method comprising administering to a subject an effective amount of a composition, lot, and/or pharmaceutical formulation described herein.

Provided herein are also methods of treating a subject having a proliferative disorder, said method comprising administering to the subject an effective amount of a composition, lot, and/or pharmaceutical formulation described above.

In some embodiments of any of the methods, the proliferative disorder is cancer. In some embodiments, the cancer is lung cancer (e.g., non-small cell lung cancer (NSCLC)), glioblastoma, pancreatic cancer, sarcoma, renal cell carcinoma, hepatocellular carcinoma, gastric cancer, colorectal cancer, and/or breast cancer. In some embodiments of any of the methods, the method further comprises administration of a second therapeutic agent. In some embodiments of any of the methods, the cell, tissue, disease associated with dysregulation of the HGF/c-met signaling axis, the proliferative and/or the cancer is characterized by c-met expression or activity. In some embodiments, c-met expression is c-met over-expression.

In addition, provided herein are articles of manufacture comprising a container with a composition, lot, or pharmaceutical formulation described above contained therein. Further provided herein are methods of making the article of manufacture.

Provided herein is a composition comprising an anti-c-met antibody, wherein host cell protein (HCP) is present in less than or equal to about 50 ng/mg, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

Also provided herein is a composition comprising an anti-c-met antibody, wherein HCP is present in less than or equal to about 50 ng/mg, the DNA levels in the composition comprising an anti-c-met antibody are less than or equal to about 0.3 pg/mg, the LpA in the composition comprising an anti-c-met antibody is less than or equal to about 2 ng/mg, the Limulus Amebocyte Lysate (LAL) in the composition comprising an anti-c-met antibody is less than or equal to about 0.01 EU/mg, the percentage of aggregates in the composition comprising an anti-c-met antibody is less than or equal to about 0.3%, the percentage of monomer in the composition comprising an anti-c-met antibody is greater than or equal to about 99.5%, the percentage of fragments in the composition comprising an anti-c-met antibody is less than or equal to about 0.3%, the percentage of acidic variants in the composition comprising an anti-c-met antibody is less than or equal to about 20%, the percentage of main peak in the composition comprising an anti-c-met antibody is greater than or equal to about 75%, and the percentage of basic variants in the composition comprising an anti-c-met antibody is less than or equal to about 2.0%, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

Also provided herein is a composition comprising an anti-c-met antibody, wherein HCP is present in less than or equal to about 15 ng/mg, the DNA levels in the composition comprising an anti-c-met antibody are less than or equal to about 0.3 pg/mg, the LpA in the composition comprising an anti-c-met antibody is less than or equal to about 2 ng/mg, the Limulus Amebocyte Lysate (LAL) in the composition comprising an anti-c-met antibody is less than or equal to about 0.01 EU/mg, the percentage of aggregates in the composition comprising an anti-c-met antibody is less than or equal to about 0.3%, the percentage of monomer in the composition comprising an anti-c-met antibody is greater than or equal to about 99.5%, the percentage of fragments in the composition comprising an anti-c-met antibody is less than or equal to about 0.3%, the percentage of acidic variants in the composition comprising an anti-c-met antibody is less than or equal to about 20%, the percentage of main peak in the composition comprising an anti-c-met antibody is greater than or equal to about 75%, and the percentage of basic variants in the composition comprising an anti-c-met antibody is less than or equal to about 2.0%, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

Also provided herein is a method of purifying an anti-c-met antibody comprising keeping a composition comprising the anti-c-met antibody at a temperature of greater than 28° C. and a pH between about pH 6 and about pH 8 for more than 6 hours, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex. In some embodiments, the method further comprises centrifuging the composition comprising the anti-c-met antibody. In some embodiments, the method further comprises loading the composition comprising the anti-c-met antibody on MabSelect SuRe resin and eluting the anti-c-met antibody.

Also provided herein is a method of purifying an anti-c-met antibody comprising loading a composition comprising an anti-c-met antibody on MabSelect SuRe resin and eluting the anti-c-met antibody, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

In some embodiments, the method further comprises loading the composition comprising the anti-c-met antibody on a weak anion exchange resin and recovering the anti-c-met antibody in the flow-through. In some embodiments, the weak anion exchange resin is run in flow-through mode.

Also provided herein is a method of purifying an anti-c-met antibody comprising loading a composition comprising an anti-c-met antibody on a weak anion exchange resin and recovering the anti-c-met antibody in the flow-through, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex. In some embodiments, the weak anion exchange resin is run in flow-through mode.

In some embodiments, the method further comprises loading the composition comprising the anti-c-met antibody on a strong cation exchange resin and eluting the anti-c-met antibody. In some embodiments, the method further comprises loading the composition comprising the anti-c-met antibody on a strong anion exchange resin and eluting the anti-c-met antibody. In some embodiments, the method further comprises ultrafiltering and/or diafiltering the composition comprising the anti-c-met antibody.

Also provided herein is a composition comprising an anti-c-met antibody purified or obtainable by any of the methods of claims 4-14, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

In some embodiments of the compositions of the invention, host cell protein (HCP) is present in less than or equal to about 50 ng/mg. In some embodiments, the HCP is present in between about 1 ng/mg and 15 ng/mg. In some embodiments, the HCP is E. coli protein (ECP).

In some embodiments of the composition and methods of the invention, the anti-c-met antibody comprises (a) a heavy chain variable domain comprising the sequence: EVQLVESGGGLVQPGGSLRLSCAASGYTFTSYWLHWVRQAPGKGLEWVGMIDPSNSDTRFNPN FKDRFTISADTSKNTAYLQMNSLRAEDTAVYYCATYRSYVTPLDYWGQGTLVTVSS (SEQ ID NO:19) and (b) a light chain variable domain comprising the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:20). In some embodiments, the Fc region increases stability of said antibody fragment compared to a Fab molecule comprising said antigen binding arm. In some embodiments, the first Fc polypeptide comprises the Fc sequence depicted in FIG. 1 (SEQ ID NO: 17) and the second Fc polypeptide comprises the Fc sequence depicted in FIG. 2 (SEQ ID NO: 18). In some embodiments, the anti-c-met antibody is onartuzumab. In some embodiments, the anti-c-met antibody binds the same epitope as onartuzumab. In some embodiments, the anti-c-met antibody has a pI of between about 8.0 and about 8.5. In some embodiments, the anti-c-met antibody is monovalent. In some embodiments, the anti-c-met antibody is an anti-c-met antibody fragment. In some embodiments, the anti-c-met antibody is a one-armed antibody.

### DESCRIPTION OF THE DRAWINGS

FIG. 1 depicts the general structures of short half-life and long half-life agonists and antagonists of c-met.

FIG. 2 depicts amino acid sequences of the framework (FR), hypervariable region (HVR), first constant domain (CL or CH1) and Fc region (Fc) of onartuzumab (MetMAb or OA5D5.v2). The Fc sequence depicted comprises “hole” (cavity) mutations T366S, L368A and Y407V, as described in WO 2005/063816.

FIG. 3 depicts sequence of an Fc polypeptide comprising “knob” (protuberance) mutation T366W, as described in WO 2005/063816. In some embodiments, an Fc polypeptide comprising this sequence forms a complex with an Fc polypeptide comprising the Fc sequence of FIG. 1 to generate an Fc region.

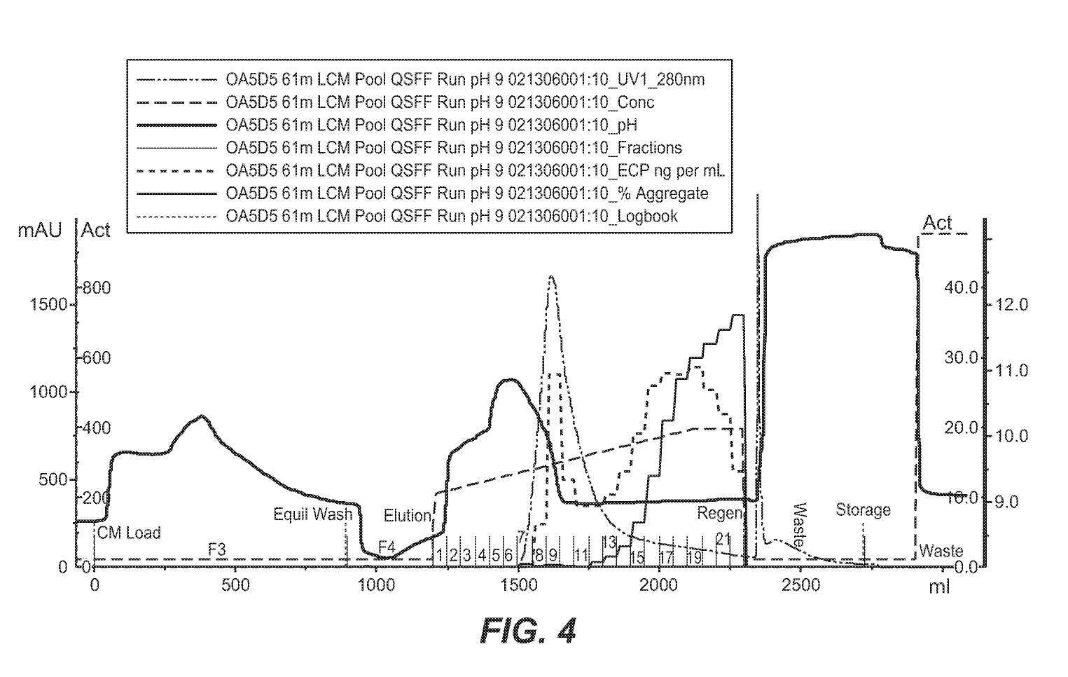

FIG. 4 depicts a chromatogram of weak CE resin pool (CM Sepharose FF) comprising onartuzumab loaded onto a strong AE resin (Q Sepharose FF) run under the gradient elution conditions.

FIG. 5A depicts the contour plot results of robot screen for Capto DEAE and onartuzumab (MetMAb) log 10 KPi (x-axis pH and y-axis ionic strength and box for operating window). FIG. 5B depicts the contour plot results of robot screen for Capto DEAE and ECP ng/mL (x-axis pH and y-axis ionic strength and blue box for operating window).

FIGS. 6A and B depict chromatograms of Capto DEAE equilibration/wash buffers using (A) Tris, NaCl equilibration/wash buffer and (B) glycine, phosphate, Tris (GPT) equilibration/wash buffer.

FIG. 7 depicts a fractional factorial multi-variate DOE performed on the Q Sepharose Fast Flow final chromatography step (x-axis conductivity mS/cm and y-axis pH).

### DETAILED DESCRIPTION

Provided herein are methods of purifying an anti-c-met antibody and compositions comprising purified anti-c-met antibodies. In some embodiments, the anti-c-met antibody is an antagonist anti-c-met antibody. In some embodiments, the anti-c-met antibody is a monovalent anti-c-met antibody (e.g., one-armed antibody). In addition, articles of manufacture comprising the purified anti-c-met antibody and uses of the compositions comprising purified anti-c-met antibody are provided.

### I. DEFINITIONS

As used herein, the terms “contaminant” or “impurity” are used interchangeably and refer to a material that is different from the desired antibody monomer product. The impurities include, but are not limited to, an antibody variant (e.g., acidic or basic antibody variant), antibody fragments, polyethyleneimine (i.e., PEI), aggregates, or derivatives of the desired antibody monomer, leached protein A, host cell impurities (e.g., ECP), lipid, nucleic acid, and/or endotoxin.

As used herein, the terms “host cell impurity” or “host cell contaminant” refer to any proteinaceous contaminant or by-product introduced by the host cell line, cell cultured fluid, and/or cell culture. Examples include, but are not limited to, Chinese Hamster Ovary Protein (CHOP), E. Coli Protein (ECP), yeast protein, simian COS protein, or myeloma cell protein (e.g., NS0 protein (mouse plastocytoma cells derived from a BALB/c mouse)). In some embodiments, the host cell impurity is ECP.

A “host cell” includes an individual cell or cell culture that can be or has been a recipient for vector(s) for incorporation of polynucleotide inserts to produce the antibody. Host cells include progeny of a single host cell, and the progeny may not necessarily be completely identical (in morphology or in genomic DNA complement) to the original parent cell due to natural, accidental, or deliberate mutation. In some embodiments, the host cell is E. coli.

As used herein, the term “monomer(s)” refers to a single unit of an antibody. For example, in the case of a one-armed antibody, a monomer consists of a) a polypeptide comprising a heavy chain and a first Fc region, b) a polypeptide comprising a light chain, and c) a polypeptide comprising a second Fc region.

As used herein, the term “aggregate(s)” refers to any multimers of an antibody or fragments thereof. For example, an aggregate can be a dimer, trimer, tetramer, or a multimer greater than a tetramer, etc.

A “buffer” is a buffered solution that resists changes in pH by the action of its acid-base conjugate components. Various buffers which can be employed depending, for example, on the desired pH of the buffer are described in Buffers. A Guide for the Preparation and Use of Buffers in Biological Systems, Mohan, C., Calbiochem Corporation (2007).

The “pH” of a solution measures the acidity or alkalinity relative to the ionization of a water sample.

The “pI” or “isoelectric point” of a molecule such as an antibody refers to the pH at which the molecule contains an equal number of positive and negative charges. The pI can be calculated from the net charge of the amino acid residues of the molecule (e.g., antibody) or can be determined by isoelectric focusing.

The term “conductivity” refers to the ability of a solution to conduct an electric current between two electrodes. The basic unit of conductivity is the siemens (S), formerly called the mho. Conductivity is commonly expressed in units of mS/cm. Since the charge on ions in solution facilities the conductance of electrical current, the conductivity of a solution is proportional to its ion concentration.

The “flow rate” is usually described as resin volumes per hour (CV/h).

The “load density” is often expressed as grams of composition processed per liter of resin.

By “binding” a molecule (e.g., antibody or contaminant) to a resin is meant exposing the molecule (e.g., antibody or contaminant) to the resin under appropriate conditions (e.g., pH and/or conductivity) such that the molecule (e.g., antibody or contaminant) is reversibly immobilized in or on the resin.

By “washing” the resin is meant passing an appropriate buffer through or over the resin.

By “eluting” a molecule (antibody or contaminant) from a resin is meant to remove the molecule therefrom.

“Flow-through” refers to binding of a first molecule (e.g., antibody or contaminant) to the resin while a second molecule (e.g., antibody or contaminant) is unretained.

The “equilibration buffer” herein is that used to prepare the resin for loading of a composition comprising the molecule of interest (e.g., antibody).

The “wash buffer” is used herein to refer to the buffer that is passed over the resin following loading and prior to elution of the molecule of interest (e.g., antibody).

The term “load density” or “loading density” is the density of the molecule of interest (e.g., antibody) (g) per liter of chromatography resin or the density of the molecule of interest (e.g., antibody) per liter of membrane/filter volume (L). In some embodiments, the loading density is measured in g/L.

The phrase “ion exchange chromatography” refers to a separation technique in which compounds are separated based on their net charge.

By “purifying” an antibody from a composition comprising the antibody and one or more contaminants is meant increasing the degree of purity of the antibody in the composition by removing (completely or partially) at least one contaminant from the composition.

An “anti-c-met antibody” and “an antibody that binds to c-met” refer to an antibody that is capable of binding c-met with sufficient affinity such that the antibody is useful as a diagnostic and/or therapeutic agent in targeting c-met. In some embodiments, the extent of binding of an anti-c-met antibody to an unrelated, non-c-met protein is less than about 10% of the binding of the antibody to c-met as measured, e.g., by a radioimmunoassay (RIA). In some embodiments, an antibody that binds to c-met has a dissociation constant (Kd) of ≦1 μM, ≦100 nM, ≦10 nM, ≦1 nM, ≦0.1 nM, ≦0.01 nM, or ≦0.001 nM (e.g., 10⁻⁸ M or less, e.g. from 10⁻⁸M to 10⁻¹³ M, e.g., from 10⁻⁹ M to 10⁻¹³ M). In some embodiments, an anti-c-met antibody binds to an epitope of c-met that is conserved among c-met from different species.

The term “antibody” is used in the broadest sense and specifically covers monoclonal antibodies (including full length monoclonal antibodies), polyclonal antibodies, multispecific antibodies (e.g., bispecific antibodies), monovalent antibodies, multivalent antibodies, and antibody fragments so long as they exhibit the desired biological activity (e.g., Fab and/or single-armed antibodies).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called α, δ, ε, γ, and μ, respectively.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full length antibody,” “intact antibody,” and “whole antibody” are used herein interchangeably to refer to an antibody having a structure substantially similar to a native antibody structure or having heavy chains that contain an Fc region as defined herein.

A “blocking” antibody or an “antagonist” antibody is one which significantly inhibits (either partially or completely) a biological activity of the antigen it binds.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “hypervariable region” or “HVR,” as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991).) With the exception of CDR1 in VH, CDRs generally comprise the amino acid residues that form the hypervariable loops. CDRs also comprise “specificity determining residues,” or “SDRs,” which are residues that contact antigen. SDRs are contained within regions of the CDRs called abbreviated-CDRs, or a-CDRs. Exemplary a-CDRs (a-CDR-L1, a-CDR-L2, a-CDR-L3, a-CDR-H1, a-CDR-H2, and a-CDR-H3) occur at amino acid residues 31-34 of L1, 50-55 of L2, 89-96 of L3, 31-35B of H1, 50-58 of H2, and 95-102 of H3. (See Almagro and Fransson, Front. Biosci. 13:1619-1633 (2008).) Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The phrase “N-terminally truncated heavy chain”, as used herein, refers to a polypeptide comprising parts but not all of a full length immunoglobulin heavy chain, wherein the missing parts are those normally located on the N terminal region of the heavy chain. Missing parts may include, but are not limited to, the variable domain, CH1, and part or all of a hinge sequence. Generally, if the wild type hinge sequence is not present, the remaining constant domain(s) in the N-terminally truncated heavy chain would comprise a component that is capable of linkage to another Fc sequence (i.e., the “first” Fc polypeptide as described herein). For example, said component can be a modified residue or an added cysteine residue capable of forming a disulfide linkage.

The term “Fc region”, as used herein, generally refers to a dimer complex comprising the C-terminal polypeptide sequences of an immunoglobulin heavy chain, wherein a C-terminal polypeptide sequence is that which is obtainable by papain digestion of an intact antibody. The Fc region may comprise native or variant Fc sequences. Although the boundaries of the Fc sequence of an immunoglobulin heavy chain may vary, the human IgG heavy chain Fc sequence is usually defined to stretch from an amino acid residue at about position Cys226, or from about position Pro230, to the carboxyl-terminus of the Fc sequence. However, the C-terminal lysine (Lys447) of the Fc sequence may or may not be present. The Fc sequence of an immunoglobulin generally comprises two constant domains, a CH2 domain and a CH3 domain, and optionally comprises a CH4 domain. By “Fc polypeptide” herein is meant one of the polypeptides that make up an Fc region. An Fc polypeptide may be obtained from any suitable immunoglobulin, such as IgG1, IgG2, IgG3, or IgG4 subtypes, IgA, IgE, IgD or IgM. In some embodiments, an Fc polypeptide comprises part or all of a wild type hinge sequence (generally at its N terminus). In some embodiments, an Fc polypeptide does not comprise a functional or wild type hinge sequence.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, e.g., Daëron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

The “hinge region,” “hinge sequence”, and variations thereof, as used herein, includes the meaning known in the art, which is illustrated in, for example, Janeway et al., Immuno Biology: the immune system in health and disease, (Elsevier Science Ltd., NY) (4th ed., 1999); Bloom et al., Protein Science (1997), 6:407-415; Humphreys et al., J. Immunol. Methods (1997), 209:193-202.

Unless indicated otherwise, the expression “multivalent antibody” is used throughout this specification to denote an antibody comprising three or more antigen binding sites. The multivalent antibody is preferably engineered to have the three or more antigen binding sites and is generally not a native sequence IgM or IgA antibody.

An “Fv” fragment is an antibody fragment which contains a complete antigen recognition and binding site. This region consists of a dimer of one heavy and one light chain variable domain in tight association, which can be covalent in nature, for example in scFv. It is in this configuration that the three HVRs of each variable domain interact to define an antigen binding site on the surface of the V_{H}-V_{L} dimer Collectively, the six HVRs or a subset thereof confer antigen binding specificity to the antibody. However, even a single variable domain (or half of an Fv comprising only three HVRs specific for an antigen) has the ability to recognize and bind antigen, although usually at a lower affinity than the entire binding site.

The “Fab” fragment contains a variable and constant domain of the light chain and a variable domain and the first constant domain (CH1) of the heavy chain. F(ab′)₂ antibody fragments comprise a pair of Fab fragments which are generally covalently linked near their carboxy termini by hinge cysteines between them. Other chemical couplings of antibody fragments are also known in the art.

The phrase “antigen binding arm”, as used herein, refers to a component part of an antibody fragment that has an ability to specifically bind a target molecule of interest. Generally and preferably, the antigen binding arm is a complex of immunoglobulin polypeptide sequences, e.g., HVR and/or variable domain sequences of an immunoglobulin light and heavy chain.

“Single-chain Fv” or “scFv” antibody fragments comprise the V_{H} and V_{L} domains of antibody, wherein these domains are present in a single polypeptide chain. Generally the Fv polypeptide further comprises a polypeptide linker between the V_{H} and V_{L} domains, which enables the scFv to form the desired structure for antigen binding. For a review of scFv, see Pluckthun in The Pharmacology of Monoclonal Antibodies, Vol 113, Rosenburg and Moore eds. Springer-Verlag, New York, pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

The expression “linear antibodies” refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “chimeric” antibody refers to an antibody in which a portion of the heavy and/or light chain is derived from a particular source or species, while the remainder of the heavy and/or light chain is derived from a different source or species.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more HVRs, compared to a parent antibody which does not possess such alterations, such alterations resulting in an improvement in the affinity of the antibody for antigen.

An antibody having a “biological characteristic” of a designated antibody is one which possesses one or more of the biological characteristics of that antibody which distinguish it from other antibodies that bind to the same antigen.

A “functional antigen binding site” of an antibody is one which is capable of binding a target antigen. The antigen binding affinity of the antigen binding site is not necessarily as strong as the parent antibody from which the antigen binding site is derived, but the ability to bind antigen must be measurable using any one of a variety of methods known for evaluating antibody binding to an antigen. Moreover, the antigen binding affinity of each of the antigen binding sites of a multivalent antibody herein need not be quantitatively the same. For the multimeric antibodies herein, the number of functional antigen binding sites can be evaluated using ultracentrifugation analysis as described in Example 2 of U.S. Patent Application Publication No. 20050186208. According to this method of analysis, different ratios of target antigen to multimeric antibody are combined and the average molecular weight of the complexes is calculated assuming differing numbers of functional binding sites. These theoretical values are compared to the actual experimental values obtained in order to evaluate the number of functional binding sites.

A “species-dependent antibody” is one which has a stronger binding affinity for an antigen from a first mammalian species than it has for a homologue of that antigen from a second mammalian species. Normally, the species-dependent antibody “binds specifically” to a human antigen (i.e. has a binding affinity (K_{d}) value of no more than about 1×10⁻⁷ M, preferably no more than about 1×10⁻⁸ M and most preferably no more than about 1×10⁻⁹ M) but has a binding affinity for a homologue of the antigen from a second nonhuman mammalian species which is at least about 50 fold, or at least about 500 fold, or at least about 1000 fold, weaker than its binding affinity for the human antigen. The species-dependent antibody can be any of the various types of antibodies as defined above. In some embodiments, the species-dependent antibody is a humanized or human antibody.

The term “substantially similar” or “substantially the same,” as used herein, refers to a sufficiently high degree of similarity between two numeric values (for example, one associated with an antibody and the other associated with a reference/comparator antibody), such that one of skill in the art would consider the difference between the two values to be of little or no biological and/or statistical significance within the context of the biological characteristic measured by said values (e.g., Kd values).

The phrase “substantially reduced” or “substantially different,” as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values).

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “pharmaceutical formulation” refers to preparations which are in such form as to permit the biological activity of the active compound(s) to be effective, and which contain no additional components which are toxic to the subjects to which the formulation is administered. “Pharmaceutically acceptable” excipients (vehicles, additives) are those which can reasonably be administered to a subject to provide an effective dose of the active compound.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

A “disorder” is any condition that would benefit from treatment with a substance/molecule or method described herein. This includes chronic and acute disorders or diseases including those pathological conditions which predispose the mammal to the disorder in question. Non-limiting examples of disorders to be treated herein include malignant and benign tumors; non-leukemias and lymphoid malignancies; neuronal, glial, astrocytal, hypothalamic and other glandular, macrophagal, epithelial, stromal and blastocoelic disorders; and inflammatory, immunologic and other angiogenesis-related disorders.

The terms “cell proliferative disorder” and “proliferative disorder” refer to disorders that are associated with some degree of abnormal cell proliferation. In one embodiment, the cell proliferative disorder is cancer.

“Tumor”, as used herein, refers to all neoplastic cell growth and proliferation, whether malignant or benign, and all pre-cancerous and cancerous cells and tissues. The terms “cancer”, “cancerous,” “cell proliferative disorder,” “proliferative disorder,” and “tumor” are not mutually exclusive as referred to herein.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer. In some embodiments, the cancer is triple-negative (ER-, PR-, HER2-) cancer. In some embodiments, the cancer is triple-negative metastatic breast cancer, including any histologically confirmed triple-negative (ER-, PR-, HER2-) adenocarcinoma of the breast with locally recurrent or metastatic disease, e.g., where the locally recurrent disease is not amenable to resection with curative intent.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis is a sequential process, contingent on tumor cells breaking off from the primary tumor, traveling through the bloodstream, and stopping at a distant site. At the new site, the cells establish a blood supply and can grow to form a life-threatening mass. Both stimulatory and inhibitory molecular pathways within the tumor cell regulate this behavior, and interactions between the tumor cell and host cells in the distant site are also significant.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result.

A “therapeutically effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

The term “anti-cancer therapy” refers to a therapy useful in treating cancer. Examples of anti-cancer therapeutic agents include, but are limited to, e.g., chemotherapeutic agents, growth inhibitory agents, cytotoxic agents, agents used in radiation therapy, anti-angiogenesis agents, apoptotic agents, anti-tubulin agents, and other agents to treat cancer, anti-CD20 antibodies, platelet derived growth factor inhibitors (e.g., Gleevec™ (Imatinib Mesylate)), a COX-2 inhibitor (e.g., celecoxib), interferons, cytokines, antagonists (e.g., neutralizing antibodies) that bind to one or more of the following targets PDGFR-beta, BlyS, APRIL, BCMA receptor(s), TRAIL/Apo2, and other bioactive and organic chemical agents, etc. Combinations thereof are also included.

An “immunoconjugate” is an antibody conjugated to one or more heterologous molecule(s), including but not limited to a cytotoxic agent.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “chemotherapeutic agent” refers to a chemical compound useful in the treatment of cancer. Examples of chemotherapeutic agents include alkylating agents such as thiotepa and cyclosphosphamide (CYTOXAN®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Nicolaou et al., Angew. Chem. Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®), liposomal doxorubicin TLC D-99 (MYOCET®), pegylated liposomal doxorubicin (CAELYX®), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoid, e.g., paclitaxel (TAXOL®), albumin-engineered nanoparticle formulation of paclitaxel (ABRAXANE™), and docetaxel (TAXOTERE®); chloranbucil; 6-thioguanine; mercaptopurine; methotrexate; platinum agents such as cisplatin, oxaliplatin (e.g., ELOXATIN®), and carboplatin; vincas, which prevent tubulin polymerization from forming microtubules, including vinblastine (VELBAN®), vincristine (ONCOVIN®), vindesine (ELDISINE®, FILDESIN®), and vinorelbine (NAVELBINE®); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); BAY439006 (sorafenib; Bayer); SU-11248 (sunitinib, SUTENT®, Pfizer); perifosine, COX-2 inhibitor (e.g. celecoxib or etoricoxib), proteosome inhibitor (e.g. PS341); bortezomib (VELCADE®); CCI-779; tipifarnib (R11577); orafenib, ABT510; Bcl-2 inhibitor such as oblimersen sodium (GENASENSE®); pixantrone; EGFR inhibitors (see definition below); tyrosine kinase inhibitors (see definition below); serine-threonine kinase inhibitors such as rapamycin (sirolimus, RAPAMUNE®); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX®), 4-hydroxytamoxifen, toremifene (FARESTON®), idoxifene, droloxifene, raloxifene (EVISTA®), trioxifene, keoxifene, and selective estrogen receptor modulators (SERMs) such as SERM3; pure anti-estrogens without agonist properties, such as fulvestrant (FASLODEX®), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN®), and nonsteroidal aromatase inhibitors such as anastrazole (ARIMIDEX®), letrozole (FEMARA®) and aminoglutethimide, and other aromatase inhibitors include vorozole (RIVISOR®), megestrol acetate (MEGASE®), fadrozole, and 4(5)-imidazoles; lutenizing hormone-releasing hormone agonists, including leuprolide (LUPRON® and ELIGARD®), goserelin, buserelin, and tripterelin; sex steroids, including progestines such as megestrol acetate and medroxyprogesterone acetate, estrogens such as diethylstilbestrol and premarin, and androgens/retinoids such as fluoxymesterone, all transretionic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

The term “prodrug” as used in this application refers to a precursor or derivative form of a pharmaceutically active substance that is less cytotoxic to tumor cells compared to the parent drug and is capable of being enzymatically activated or converted into the more active parent form. See, e.g., Wilman, “Prodrugs in Cancer Chemotherapy” Biochemical Society Transactions, 14, pp. 375-382, 615th Meeting Belfast (1986) and Stella et al., “Prodrugs: A Chemical Approach to Targeted Drug Delivery,” Directed Drug Delivery, Borchardt et al., (ed.), pp. 247-267, Humana Press (1985). The prodrugs include, but are not limited to, phosphate-containing prodrugs, thiophosphate-containing prodrugs, sulfate-containing prodrugs, peptide-containing prodrugs, D-amino acid-modified prodrugs, glycosylated prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs which can be converted into the more active cytotoxic free drug. Examples of cytotoxic drugs that can be derivatized into a prodrug form for use include, but are not limited to, those chemotherapeutic agents described above.

A “growth inhibitory agent” when used herein refers to a compound or composition which inhibits growth of a cell (e.g., a cell whose growth is dependent upon HGF/c-met activation either in vitro or in vivo). Thus, the growth inhibitory agent may be one which significantly reduces the percentage of HGF/c-met-dependent cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

By “radiation therapy” is meant the use of directed gamma rays or beta rays to induce sufficient damage to a cell so as to limit its ability to function normally or to destroy the cell altogether. It will be appreciated that there will be many ways known in the art to determine the dosage and duration of treatment. Typical treatments are given as a one time administration and typical dosages range from 10 to 200 units (Grays) per day.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

The term “package insert” is used to refer to instructions customarily included in commercial packages of therapeutic products, that contain information about the indications, usage, dosage, administration, combination therapy, contraindications and/or warnings concerning the use of such therapeutic products.

It is understood that aspect and embodiments of the invention described herein include “consisting” and/or “consisting essentially of” aspects and embodiments.

As used herein, the singular form “a”, “an”, and “the” includes plural references unless indicated otherwise.

As is understood by one skilled in the art, reference to “about” a value or parameter herein includes (and describes) embodiments that are directed to that value or parameter per se. For example, description referring to “about X” includes description of “X”.

### II. METHOD OF PURIFICATION AND PURIFIED COMPOSITIONS

Provided herein are methods of purifying an anti-c-met antibody and compositions comprising a purified anti-c-met antibody. In some embodiments, the anti-c-met antibody is produced in E. coli. In some embodiments, the anti-c-met antibody is onartuzumab.

In particular, provided herein are methods of purifying a composition comprising an anti-c-met antibody comprising keeping a composition comprising the anti-c-met antibody at a temperature of greater than 28° C. and a pH between about pH 6 and about pH 8 for more than 6 hours. The keeping of a composition comprising the anti-c-met antibody at a temperature of greater than 28° C. and a pH between about pH 6 and about pH 8 for more than 6 hours is referred to herein as the “flocculation step.” In some embodiments, the composition comprising the anti-c-met antibody further comprises a cationic polymer. In some embodiments, the cationic polymer is PEI. In some embodiments, the PEI concentration (in the composition) is 0.1% (v/v), 0.1% (v/v), 0.2% (v/v), 0.25% (v/v), 0.3% (v/v), 0.35% (v/v), 0.4% (v/v), 0.45% (v/v), or 0.5% (v/v). In some embodiment, the PEI concentration is about any of 0.1%-0.4% (v/v), 0.2%-0.6% (v/v), 0.2%-0.4% (v/v). In some embodiments, the PEI concentration is about 0.2% (v/v). In some embodiments, the PEI concentration is about 0.4% (v/v). For example, provided herein are methods of purifying a composition comprising an anti-c-met antibody and PEI comprising keeping a composition comprising the anti-c-met antibody at a temperature of greater than 28° C. and a pH between about pH 6 and about pH 8 for more than 6 hours. In some embodiments, the method further comprises a) centrifugation and/or b) dilution and centrifugation and/or c) dilution, centrifugation and filtration.

In some embodiments, the composition comprising the anti-c-met antibody in the flocculation step is kept at a temperature between about any of 28° C.-32° C., 28° C.-31° C., 28° C.-30° C., 29° C.-32° C., 29° C.-31° C., 28° C.-34° C., 28° C.-35° C., 30° C.-34° C., 30° C.-35° C. In some embodiments, the composition comprising the anti-c-met antibody in the flocculation step is kept at a temperature of about any of 28° C., 29° C., 30° C., 31° C., 32° C., 33° C., 34° C., 35° C., or 36° C.

In some embodiments, the composition comprising the anti-c-met antibody in the flocculation step is at a pH between about any of 6-7, 6-7.5, 6.5-8, 6.5-7.5, or 6.5-7. In some embodiments, the composition comprising the anti-c-met antibody in the flocculation step is at a pH between about any of 6, 6.2, 6.4, 6.5, 6.6, 6.8, 7, 7.2, 7.4, 7.5, 7.6, 7.8, or 8.

In some embodiments, the composition comprising the anti-c-met antibody in the flocculation step is kept at a temperature described above and/or pH described above for greater than about any of 6.5, 7, 8, 9, 10, 11, 12, 13, 14, 15, 16, 17, 18, 19, or 20 hours. In some embodiments, the composition comprising the anti-c-met antibody in the flocculation step is kept at a temperature described above and/or pH described above for about any of 6.5, 7, 8, 9, 10, 11, 12, 13, 14, 15, 16, 17, 18, 19, or 20 hours. In some embodiments, the composition comprising the anti-c-met antibody in the flocculation step is kept at a temperature described above and/or pH described above for between about any of 6-48, 6-24, 6-20, 6-12, 6-15, 6-16, 6-18, 6-10, or 6-8 hours. In some embodiments, the composition comprising the anti-c-met antibody in the flocculation step is kept at a temperature described above and/or pH described above for about any of 6.5, 7, 8, 9, 10, 11, 12, 13, 14, 15, 16, 17, 18, 19, or 20 hours. In some embodiments, the composition comprising the anti-c-met antibody further comprises a cationic polymer. In some embodiments, the cationic polymer is PEI. In some embodiments, the PEI concentration (in the composition) is 0.1% (v/v), 0.1% (v/v), 0.2% (v/v), 0.25% (v/v), 0.3% (v/v), 0.35% (v/v), 0.4% (v/v), 0.45% (v/v), or 0.5% (v/v). In some embodiment, the PEI concentration is about any of 0.1%-0.4% (v/v), 0.2%-0.6% (v/v), 0.2%-0.4% (v/v). In some embodiments, the PEI concentration is about 0.2% (v/v). In some embodiments, the PEI concentration is about 0.4% (v/v).

In some embodiments, the composition comprising the anti-c-met antibody in the flocculation step is kept at a temperature of about 28° C., and a pH of about 6, for about 12, 14, 16, 18, 20 or 22 hours. In some embodiments, the composition comprising the anti-c-met antibody in the flocculation step is kept at a temperature of about 30° C., and a pH of about 6, for about 12, 14, 16, 18, 20 or 22 hours. In some embodiments, the composition comprising the anti-c-met antibody in the flocculation step is kept at a temperature of about 34° C., and a pH of about 6, for about 12, 14, 16, 18, 20 or 22 hours. In some embodiments, the composition comprising the anti-c-met antibody further comprises a cationic polymer. In some embodiments, the cationic polymer is PEI. In some embodiments, the PEI concentration (in the composition) is 0.1% (v/v), 0.1% (v/v), 0.2% (v/v), 0.25% (v/v), 0.3% (v/v), 0.35% (v/v), 0.4% (v/v), 0.45% (v/v), or 0.5% (v/v). In some embodiment, the PEI concentration is about any of 0.1%-0.4% (v/v), 0.2%-0.6% (v/v), 0.2%-0.4% (v/v). In some embodiments, the PEI concentration is about 0.2% (v/v). In some embodiments, the PEI concentration is about 0.4% (v/v). In some embodiments, the cationic polymer is PEI at a concentration of about 0.6% (v/v).

In some embodiments, the composition comprising the anti-c-met antibody and a cationic polymer in the flocculation step is kept at a temperature of about 28° C., and a pH of about 6, for about 12, 14, 16, 18, 20 or 22 hours. In some embodiments, the composition comprising the anti-c-met antibody and a cationic polymer in the flocculation step is kept at a temperature of about 30° C., and a pH of about 6, for about 12, 14, 16, 18, 20 or 22 hours. In some embodiments, the composition comprising the anti-c-met antibody and a cationic polymer in the flocculation step is kept at a temperature of about 34° C., and a pH of about 6, for about 12, 14, 16, 18, 20 or 22 hours. In some embodiments, the cationic polymer is PEI at a concentration of about 0.2% (v/v). In some embodiments, the cationic polymer is PEI at a concentration of about 0.4% (v/v). In some embodiments, the cationic polymer is PEI at a concentration of about 0.6% (v/v).

In some embodiments, the composition comprising the anti-c-met antibody and a cationic polymer in the flocculation step is kept at a temperature of about 28° C., and a pH of about 6, for greater than or equal to about 16 or 20 hours. In some embodiments, the composition comprising the anti-c-met antibody and a cationic polymer in the flocculation step is kept at a temperature of about 30° C., and a pH of about 6, for greater than or equal to about 16 or 20 hours. In some embodiments, the composition comprising the anti-c-met antibody and a cationic polymer in the flocculation step is kept at a temperature of about 34° C., and a pH of about 6, for greater than or equal to about 16 or 20 hours. In some embodiments, the cationic polymer is PEI at a concentration of about 0.2% (v/v). In some embodiments, the cationic polymer is PEI at a concentration of about 0.4% (v/v). In some embodiments, the cationic polymer is PEI at a concentration of about 0.6% (v/v).

The use of the flocculation step in the purification of an anti-c-met antibody may result in one or more improvements provided below. In some embodiments, keeping the composition comprising the anti-c-met antibody at a temperature of greater than 28° C. and a pH between about pH 6 and about pH 8 for more than 6 hours improves flocculation effectiveness (e.g., compared to a method of purification in the absence of the flocculation step). In some embodiments, keeping the composition comprising the anti-c-met antibody at a temperature of greater than 28° C. and a pH between about pH 6 and about pH 8 for more than 6 hours leads to better centrifugation separation (e.g., compared to a method of purification in the absence of the flocculation step). In some embodiments, keeping the composition comprising the anti-c-met antibody at a temperature of greater than 28° C. and a pH between about pH 6 and about pH 8 for more than 6 hours leads to better centrate and/or protein A pool stability (e.g., compared to a method of purification in the absence of the flocculation step). In some embodiments, keeping the composition comprising the anti-c-met antibody at a temperature of greater than 28° C. and a pH between about pH 6 and about pH 8 for more than 6 hours results in improved stability such that the centrate and/or protein A pools can be held at 15° C.-25° C. (e.g., about any of 15° C., 20° C., or 25° C.) In some embodiments, keeping the composition comprising the anti-c-met antibody at a temperature of greater than 28° C. and a pH between about pH 6 and about pH 8 for more than 6 hours improves filtration for centrate, protein A load, and/or later chromatography steps (e.g., compared to a method of purification in the absence of the flocculation step). In some embodiments, keeping the composition comprising the anti-c-met antibody at a temperature of greater than 28° C. and a pH between about pH 6 and about pH 8 for more than 6 hours reduces impurities including, but not limited to, DNA and HCP, such as ECP, (e.g., compared to a method of purification in the absence of the flocculation step). In some embodiments, keeping the composition comprising the anti-c-met antibody at a temperature of greater than 28° C. and a pH between about pH 6 and about pH 8 for more than 6 hours allows for additional dilution(s) to reduce percent solids content (e.g., compared to a method of purification in the absence of the flocculation step). In some embodiments, the additional dilution(s) improve centrifuge yield (e.g., compared to the same method in the absence of the flocculation step). In some embodiments, keeping the composition comprising the anti-c-met antibody at a temperature of greater than 28° C. and a pH between about pH 6 and about pH 8 for more than 6 hours increases centrifuge flow rate (e.g., compared to the same method in the absence of the flocculation step). In some embodiments, the increase in centrifuge flow rate allows for shorter processing time and substantially equivalent separation (e.g., compared to the same method in the absence of the flocculation step). In some embodiments, keeping the composition comprising the anti-c-met antibody at a temperature of greater than 28° C. and a pH between about pH 6 and about pH 8 for more than 6 hours improves flocculation effectiveness (e.g., compared to a method of purification in the absence of the flocculation step). In some embodiments, the composition comprising the anti-c-met antibody further comprises a cationic polymer. In some embodiments, the cationic polymer is PEI. In some embodiments, the PEI concentration (in the composition) is 0.1% (v/v), 0.1% (v/v), 0.2% (v/v), 0.25% (v/v), 0.3% (v/v), 0.35% (v/v), 0.4% (v/v), 0.45% (v/v), or 0.5% (v/v). In some embodiment, the PEI concentration is about any of 0.1%-0.4% (v/v), 0.2%-0.6% (v/v), 0.2%-0.4% (v/v). In some embodiments, the PEI concentration is about 0.2% (v/v). In some embodiments, the PEI concentration is about 0.4% (v/v).

In some embodiments, the use of the flocculation step in the purification of an anti-c-met antibody may result in any one or more of the improvements when the composition comprising the anti-c-met antibody is kept at a temperature of 30° C. or greater and a pH of about pH 6 for more than 6 hours, e.g., for about 10, 12, 14, 16, 18, 20, 22, or 24 hours. In some embodiments, the composition is kept at a temperature of 30° C. or greater and a pH of about pH 6 for about 16 hours or longer. In some embodiments, the composition is kept at a temperature of 30° C. or greater and a pH of about pH 6 for about 10 hours or longer. In some embodiments, the composition is kept at a temperature of 30° C. or greater and a pH of about pH 6 for about 12 hours or longer. In some embodiments, the composition comprising the anti-c-met antibody further comprises a cationic polymer. In some embodiments, the cationic polymer is PEI. In some embodiments, the PEI concentration (in the composition) is 0.1% (v/v), 0.1% (v/v), 0.2% (v/v), 0.25% (v/v), 0.3% (v/v), 0.35% (v/v), 0.4% (v/v), 0.45% (v/v), or 0.5% (v/v). In some embodiment, the PEI concentration is about any of 0.1%-0.4% (v/v), 0.2%-0.6% (v./v), 0.2%-0.4% (v/v). In some embodiments, the PEI concentration is about 0.2% (v/v). In some embodiments, the PEI concentration is about 0.4% (v/v).

In some embodiments, the method further comprises centrifugation. In some embodiments, the method further comprises affinity chromatography (e.g., protein A affinity chromatography) such as those described below. In some embodiments, the method further comprises one or more ion-exchange chromatography steps such as any of those described below. In some embodiments, the method further comprises ultrafiltration and/or diafiltration. The steps of the method of purifying the anti-c-met antibody can be completed in any order. In some embodiments, the method comprises a) the flocculation step and centrifugation (e.g., 6000 rpm, 20 lpm, Q/σ=6×10⁻³ L/hr/m²) followed by b) affinity chromatography (e.g., protein A affinity chromatography).

In some embodiments, the method further comprises filtration (e.g., after centrifugation). In some embodiments, filtration is depth filtration.

In some embodiments, the composition comprising the anti-c-met antibody is generated by homogenization of a cell culture. In some embodiments, the cell culture is E coli cell culture. In some embodiment, the cell culture is homogenized, whereby the resulting composition comprising the anti-c-met antibody comprises about 8-20 percent solids.

In addition, provided herein are methods of purifying a composition comprising an anti-c-met antibody using affinity chromatography (e.g., protein A affinity chromatograph). In some embodiments, the method comprises loading a composition comprising the anti-c-met antibody on protein A resin. In some embodiments, the method comprises loading a composition comprising the anti-c-met antibody on protein A resin and eluting the anti-c-met antibody.

Examples of protein A resins include, but are not limited to MabSelect™, MabSelect Sure™ Prosep vA, Prosep Ultra-Plus, and/or POROS MabCapture A. In some embodiments, the protein A resin comprises an agarose matrix. In some embodiments, the protein A resin comprising an agarose matrix is MabSelect SuRe™ and MabSelect™. In some embodiments, the protein A resin is MabSelect SuRe™ resin (GE Healthcare (Piscataway, N.J.); a resin comprising an alkali-tolerant protein A-derived ligand bound to an agarose matrix). For example, in some embodiments, the method comprises loading a composition comprising the anti-c-met antibody on MabSelect SuRe™ resin and eluting the anti-c-met antibody.

In some embodiments, the flow rate for protein A affinity chromatography is between about any of 5-40 CV/hour, 15-40 CV/hour, 20-40 CV/hour, or 25-40 CV/hour.

The protein A resin can be equilibrated with an equilibration buffer, and the unpurified and/or partially purified anti-c-met antibodies comprising various impurities (e.g., harvested cell proteins (e.g., ECP)) can then be loaded onto the equilibrated resin. As the anti-c-met antibodies flow through the resin, the anti-c-met antibodies and various impurities are adsorbed to the immobilized protein A. The wash buffers can be used to remove some impurities, such as host cell impurities, but not anti-c-met antibodies. The anti-c-met antibodies are eluted from the resin with the elution buffer.

The equilibration buffer for protein A affinity chromatography may comprise Tris and a salt. Examples of useful salts include, but are not limited to, sodium chloride, sodium sulfate, magnesium sulfate, and/or potassium chloride. In some embodiments, the salt is potassium chloride. In some embodiments, the salt is sodium chloride. In some embodiments, the concentration of Tris in the equilibration buffer is between about 0.01 M and about 0.1 M. For example, in some embodiments, the concentration of Tris is about any of 0.01 M, 0.025 M, 0.05 M, 0.075 M, or 0.1 M. In some embodiments, the concentration of salt is between about 0.01 M and about 0.1 M. For example, in some embodiments, the concentration of salt is about any of 0.01 M, 0.025 M, 0.05 M, 0.075 M, or 0.1 M. In some embodiments, the pH of the equilibration buffer is about any of 7.1, 7.3, 7.5, 7.7, or 7.9.

The wash buffer for protein A affinity chromatography may comprise a buffer. Examples of useful buffers include, but are not limited to, arginine buffers, acetate buffers, citrate buffers, and/or phosphate buffers. In some embodiments, the buffer is a phosphate buffer. In some embodiments, the phosphate buffer is potassium phosphate. In some embodiments, the phosphate buffer is sodium phosphate. In some embodiments, the concentration of phosphate buffer is between about 0.1 M and about 1.0 M. For example, in some embodiments, the concentration of phosphate buffer is about any of 0.2 M, 0.4 M, 0.6 M, 0.8 M, or 0.1 M. In some embodiments, the pH of the wash buffer is about any of 7.0, 7.25, 7.5, 7.75, or 8.0.

The elution buffer for protein A affinity chromatography may comprise a buffer. Examples of useful buffers include, but are not limited to, arginine buffers, acetate buffers, citrate buffers, and/or phosphate buffers. In some embodiments, the buffer is a phosphate buffer. In some embodiments, the phosphate buffer is potassium phosphate. In some embodiments, the phosphate buffer is sodium phosphate. In some embodiments, the phosphate buffer is glycine phosphate. In some embodiments, the concentration of phosphate buffer is between about 0.01 M and about 0.1 M. For example, in some embodiments, the concentration of phosphate buffer is about any of 0.01 M, 0.025 M, 0.05 M, 0.075 M, or 0.1 M. In some embodiments, the pH of the elution buffer is about any of 3.1, 3.3, 3.5, or 3.7. In some embodiments, the conductivity of the elution buffer is between about 0.9 mS/cm and about 1.1 mS/cm. In some embodiments, the conductivity of the elution buffer is about any of 0.9 mS/cm, 1.0 mS/cm, or 1.1 mS/cm. For example, in some embodiments, the method comprises loading a composition comprising the anti-c-met antibody on a protein A affinity resin (e.g., MabSelect SuRe™ resin) and eluting the anti-c-met antibody with an elution buffer, wherein the elution buffer comprises a glycine phosphate at a concentration of about 0.075 M and conductivity of between about 0.9 mS/cm and about 1.1 mS/cm. MabSelect SuRe™ resin is a highly cross-linked agarose matrix coupled via epoxy activation to an alkali-tolerant recombinant protein A ligand.

In some embodiments, the method further comprises a flocculation step such as those described above. In some embodiments, the method further comprises centrifugation. In some embodiments, the method further comprises one or more ion-exchange chromatography steps such as any of those described herein. In some embodiments, the method further comprises ultrafiltration and/or diafiltration. The steps of the method of purifying the anti-c-met antibody can be completed in any order. In some embodiments, the method comprises a) the flocculation step and centrifugation followed by b) protein A affinity chromatography (e.g., MabSelect SuRe™ resin) followed by c) one or more ion-exchange chromatography. In some embodiments, the anti-c-met antibody is produced in E. coli. In some embodiments, the anti-c-met antibody is onartuzumab.

In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading a composition comprising the anti-c-met antibody on a protein A affinity resin (e.g., MabSelect SuRe™ resin), and d) eluting the anti-c-met antibody from the protein A affinity resin, wherein the HCP (e.g., average HCP) is reduced to less than 1,800 ng/mg. In some embodiments, the HCP (e.g., average HCP) is reduced to less than about any of 1,700 ng/mg, 1,600 ng/mg, 1,500 ng/mg, 1,400 ng/mg, 1,300 ng/mg, 1,200 ng/mg, 1,100 ng/mg, or 1,000 ng/mg. In some embodiments, the HCP (e.g., average HCP) is reduced to between about 800 ng/mg and about 1,200 ng/mg or between about 900 ng/mg and about 1,100 ng/mg. In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading a composition comprising the anti-c-met antibody on MabSelect SuRe™ resin, and d) eluting the anti-c-met antibody from the protein A affinity resin, and wherein the HCP (e.g., average HCP) is reduced by greater than about any of 40%, 35%, 30%, 25%, or 20% compared to the same method of purification in the absence of the flocculation step and/or the same method of purification in the absence of the flocculation step and Prosep vA as the protein A affinity chromatography resin. In some embodiments, the anti-c-met antibody is produced in E. coli. In some embodiments, the HCP and/or average HCP is ECP and/or average ECP. In some embodiments, the anti-c-met antibody is onartuzumab.

In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading a composition comprising the anti-c-met antibody on a protein A affinity resin (e.g., MabSelect SuRe™ resin), and d) eluting the anti-c-met antibody from the protein A affinity resin, and wherein the PEI after protein A affinity chromatography is reduced to less than about any of 50 μg/mL, 45 μg/mL, 40 μg/mL, 35 μg/mL, or 30 μg/mL. In some embodiments, the PEI after protein A affinity chromatography is undetectable. In some embodiments, the protein A affinity resin is an agarose matrix. In some embodiments, the anti-c-met antibody is produced in E. coli. In some embodiments, the anti-c-met antibody is onartuzumab.

Further provided herein are methods of purifying a composition comprising an anti-c-met antibody comprising one or more ion exchange chromatography steps. In some embodiments, the ion exchange chromatography is anion exchange (AE) chromatography. In some embodiments, the ion exchange chromatography is cation exchange (CE) chromatography.

Provided herein, for example, are methods of purifying a composition comprising an anti-c-met antibody comprising loading a composition comprising the anti-c-met antibody on a weak AE resin and recovering the anti-c-met antibody in the flow-through. In some embodiments, the weak AE resin is run in flow-through mode. In some embodiments, the anti-c-met antibody is produced in E. coli. In some embodiments, the anti-c-met antibody is onartuzumab.

Weak AE resins generally contain a tertiary or secondary amine functional group, such as DEAE (diethylaminoethyl). Examples of weak AE resins are known in the art and include, but are not limited to, DEAE Sepharose Fast Flow, Capto DEAE, POROS D, Toyopearl DEAE 650C, Toyopearl DEAE 650M, Toyopearl DEAE 650S, TSKgel DEAE 5PW 30, and/or TSKgel DEAE 5PW 20. In some embodiments, the weak AE resin is Capto DEAE (a weak diethylaminoethyl anion exchanger attached to a chemically modified, high-flow agarose matrix). In some embodiments, the weak AE resin is DEAE Sepharose Fast Flow.

In some embodiments, the flow rate for the weak AE chromatography is about any of 100 cm/hour, 125 cm/hour, 150 cm/hour, 175 cm/hour, 250 cm/hour, 500 cm/hour, 750 cm/hour, 1000 cm/hour, 1250 cm/hour, or 1400 cm/hour.

The weak AE resin can be equilibrated with an equilibration buffer, and the unpurified or partially purified anti-c-met antibodies comprising various impurities (e.g., harvested cell proteins (e.g., ECP)) can then be loaded onto the equilibrated resin. As the anti-c-met antibodies flow through the resin, the impurities are adsorbed to the weak AE resin while the anti-c-met antibodies are present in the flow-through.

The equilibration buffer for the weak AE chromatography includes, but is not limited to, Tris buffers, glycine buffers, CAPSO, CAPS, CHES, TAPS, and/or phosphate buffers. In some embodiments, the equilibration buffer for the weak AE chromatography comprises Tris and a salt. Examples of salts useful in the equilibration buffer include, but are not limited to, sodium chloride, sodium sulfate, magnesium sulfate, and/or potassium chloride. In some embodiments, the salt is potassium chloride. In some embodiments, the salt is sodium chloride. In some embodiments, the equilibration buffer for the weak AE chromatography comprises glycine, phosphate, and Tris. In some embodiments, the concentration of Tris in the equilibration buffer is between about 0.01 M and about 0.15 M or between about 0.01 M and about 0.1M. For example, in some embodiments, the concentration of Tris is about any of 0.01 M, 0.025 M, 0.05 M, 0.075 M, or 0.1M. In some embodiments, the concentration of salt is between about 0.001 M and 0.01 M. For example, in some embodiments, the concentration of salt is about any of 0.001 M, 0.0025 M, 0.005 M, 0.0075 M, or 0.01 M. In some embodiments, the concentration of glycine is between about 25-100 mM. In some embodiments, the concentration of phosphoric acid is about any of 2.5 mM, 5.0 mM, 7.5 mM, or 10.0 mM. In some embodiments, the concentration of phosphoric acid is between about 2.5-10.0 mM. In some embodiments, the concentration of glycine is about any of 25 mM, 50 mM, 75 mM, or 100 mM. In some embodiments, the pH of the equilibration buffer is higher than the pI of the polypeptide of interest (e.g., anti-c-met antibody). In some embodiments, the pH of the equilibration buffer is between about 8.7 and about 9.1. In some embodiments, the pH of the equilibration buffer is about any of 8.7, 8.8, 8.9, or 9.0. In some embodiments, the pH higher than the pI of the polypeptide of interest (e.g., anti-c-met antibody) causes a net negative charge on the polypeptide of interest. In some embodiments, the net negative charge on the polypeptide of interest (e.g., anti-c-met antibody) results in an attractive force between the polypeptide of interest and the weak anion resin. In some embodiments, the polypeptide of interest (e.g., anti-c-met antibody) has a pI of between about 8.2 and 8.4 (e.g., about, 8.2, about 8.3, and/or about 8.4).

In some embodiments, the method further comprises a flocculation step such as described above. In some embodiments, the method further comprises centrifugation. In some embodiments, the method further comprises protein A affinity chromatography as described above. In some embodiments, the method further comprises one or more additional ion-exchange chromatography steps such as any of those described herein. In some embodiments, the method further comprises ultrafiltration and/or diafiltration. In some embodiments, the method comprises a) a flocculation step, b) a centrifugation step followed by c) affinity chromatography (e.g., protein A affinity chromatography) followed by d) weak anion exchange chromatography. In some embodiments, provided herein methods of purifying a composition comprising an anti-c-met antibody comprising a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), and d) eluting the anti-c-met antibody from the protein A affinity resin, d) loading the composition comprising the anti-c-met antibody on a weak AE resin (e.g., DEAE Sepharose Fast Flow or Capto DEAE) and e) recovering the anti-c-met antibody in the flow-through from the weak AE resin. The steps of the method of purifying the anti-c-met antibody can be completed in any order. In some embodiments, the steps are done sequentially. In some embodiments, the anti-c-met antibody is produced in E. coli. In some embodiments, the anti-c-met antibody is onartuzumab.

In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE) and f) recovering the anti-c-met antibody in the flow-through from the weak AE resin, and wherein the HCP (e.g., average HCP) is reduced to less than about 200 ng/mg. In some embodiments, the HCP (e.g., average HCP) is reduced to less than or equal to about any of 300 ng/mg, 275 ng/mg, 250 ng/mg, 225 ng/mg, 200 ng/mg, 190 ng/mg, 180 ng/mg, or 170 ng/mg. In some embodiments, the HCP (e.g., average HCP) is reduced to between about 150 ng/mg and about 190 ng/mg or between about 160 ng/mg and about 180 ng/mg. In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE) and f) recovering the anti-c-met antibody in the flow-through from the weak AE resin, and wherein the HCP (e.g., average HCP) is reduced by greater than about 75%, 70%, 65%, 60%, or 55% compared to the same method in the absence of the flocculation step, Prosep vA as the protein A affinity chromatography resin, and/or a weak CE resin (e.g., CM Sepharose). In some embodiments, the steps are done sequentially. In some embodiments, the anti-c-met antibody is produced in E. coli. In some embodiments, the HCP and/or average HCP is ECP and/or average ECP. In some embodiments, the anti-c-met antibody is onartuzumab.

In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin) d) eluting the anti-c-met antibody from the protein A affinity resin, e) loading a composition comprising the anti-c-met antibody on a weak AE resin (e.g., DEAE Sepharose Fast Flow or Capto DEAE) and f) recovering the anti-c-met antibody in the flow-through from the weak AE resin, and wherein the HCP (e.g., average HCP) is reduced to less than about 200 ng/mg. In some embodiments, the HCP (e.g., average HCP) is reduced to less than or equal to about any of 300 ng/mg, 275 ng/mg, 250 ng/mg, 225 ng/mg, 200 ng/mg, 190 ng/mg, 180 ng/mg, or 170 ng/mg. In some embodiments, the HCP (e.g., average HCP) is reduced to between about 150 ng/mg and about 190 ng/mg or between about 160 ng/mg and about 180 ng/mg. In some embodiments, the method comprises a) keeping the composition comprising the anti-c-met antibody at a temperature of greater than 28° C. and a pH between about pH 6 and about pH 8 for more than 6 hours b) centrifuging the composition comprising the anti-c-met antibody, c) loading the composition comprising the anti-c-met antibody on a protein A affinity resin (e.g., MabSelect SuRe™ resin) d) eluting the anti-c-met antibody from the protein A affinity resin, e) loading a composition comprising the anti-c-met antibody on a weak AE resin (e.g., DEAE Sepharose Fast Flow or Capto DEAE), and f) recovering the anti-c-met antibody in the flow-through from the weak AE resin, and wherein the HCP (e.g., average HCP) is reduced by greater than about 75%, 70%, 65%, 60%, or 55% compared to the same method in the absence of the flocculation step, Prosep vA as the protein A affinity chromatography resin, and/or a weak CE resin (e.g., CM Sepharose). In some embodiments, the steps are done sequentially. In some embodiments, the anti-c-met antibody is produced in E. coli. In some embodiments, the HCP and/or average HCP is ECP and/or average ECP. In some embodiments, the anti-c-met antibody is onartuzumab.

In some embodiments of any of the methods of purification described, the method further comprises loading a composition comprising the anti-c-met antibody on a strong CE resin and eluting the anti-c-met antibody. In some embodiments, the anti-c-met antibody is produced in E. coli. In some embodiments, the anti-c-met antibody is onartuzumab.

Strong CE exchange resins generally contain a sulfonium ion. Examples of strong CE resins are known in the art and include, but are not limited to, MiniS PC 3.2/3, Mini S 4.6/50 PE, Mono S 5/50GL, RESOURCE S, SOURCE 15S, SOURCE 30S, SP Sepharose Fast Flow, POROS HS 50, MacroCap SP, HiTrap SPFF, HiTrap Capto S, SP Sepharose XL, Toyopearl SP 550c, SP Sepharose BB, TSKGel SP-5PW-HR20, Toyopearl SP 650c, Toyopearl MegaCap II SP-550EC, Toyopearl SP-550C, Toyopearl GigaCap S-650M, Toyopearl SP-650M, Toyopearl SP-650S, TSKgel SP-3PW 30, TSKgel SP 5P@ 30, TSKgel SP-5PW 20, Capto S, and/or Fractogel SO3. In some embodiments, the strong CE resin is POROS HS 50 (sulfopropyl surface functionality attached to a crosslinked poly(styrene-divinylbenzene) support matrix). In some embodiments, the strong CE resin is SP Sepharose Fast Flow. In some embodiments, the strong CE resin is Toyopearl SP 550c

In some embodiments, the flow rate for the strong CE chromatography is between about any of 50-500 cm/hr, 50-250 cm/hr, and/or 250-500 cm/hour. In some embodiments, the flow rate is about any of 105 cm/hour, 125 cm/hour, 135 cm/hour, 145 cm/hour, 155 cm/hour, 165 cm/hour, 185 cm/hr, and/or 250 cm/hr.

In some embodiments, the conductivity for the strong CE chromatography is less than about 1.9 mS/cm at about pH 8.9-9.0 and/or less than about 2.4 mS/cm at pH 9.0 or greater. In some embodiments, the conductivity is between about 1.4 mS/cm and about 1.9 mS/cm at about pH 8.9-pH 9.0 or between about 1.4 mS/cm and about 1.9 mS/cm at about pH 8.9-pH 9.5.

The strong CE resin can be equilibrated with an equilibration buffer, and the unpurified or partially purified anti-c-met antibodies comprising various impurities (e.g., harvested cell proteins (e.g., ECP)) can then be loaded onto the equilibrated resin. As the anti-c-met antibodies flow through the resin, the anti-c-met antibodies and various impurities are adsorbed to the immobilized strong CE resin. The wash buffers can be used to remove some impurities, such as host cell impurities, but not anti-c-met antibodies. In some embodiments, the equilibration buffer is utilized as the wash buffer. The anti-c-met antibodies are eluted from the resin with the elution buffer.

The equilibration buffer for the strong CE chromatography may comprise MOPS. In some embodiments, the concentration of MOPS in the equilibration buffer is between about 0.01 M and about 0.1 M. For example, in some embodiments, the concentration of MOPS is about any of 0.01 M, 0.025 M, 0.05 M, 0.075 M, or 0.1M. In some embodiments, the pH of the equilibration buffer is about any of 7.0, 7.1, 7.2, 7.3, or 7.4.

The elution buffer for the strong CE chromatography may comprise MOPS and an acetate salt. In some embodiments, the salt is potassium acetate. In some embodiments, the salt is sodium acetate. In some embodiments, the concentration of MOPS in the equilibration buffer is between about 0.01 M and about 0.1 M. For example, in some embodiments, the concentration of MOPS is about any of 0.01 M, 0.025 M, 0.05 M, 0.075 M, or 0.1 M. In some embodiments, the concentration of the acetate salt is about any of 0.1 M, 0.15 M, 0.2 M, 0.25 M, or 0.3 M. In some embodiments, the pH of the equilibration buffer is about any of 7.0, 7.1, 7.2, 7.3, or 7.4.

In some embodiments, the method further comprises a flocculation step such as described above. In some embodiments, the method further comprises centrifugation. In some embodiments, the method further comprises protein A affinity chromatography as described above. In some embodiments, the method further comprises one or more additional ion-exchange chromatography steps such as any of those described herein. In some embodiments, the method further comprises ultrafiltration and/or diafiltration. In some embodiments, the method comprises a) the flocculation step followed by b) centrifugation step followed by c) affinity chromatography (e.g., protein A affinity chromatography) followed by d) weak anion exchange chromatography followed by e) strong cation exchange chromatography. For example, in some embodiments, methods of purifying a composition comprising an anti-c-met antibody comprising a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE), f) recovering the anti-c-met antibody in the flow-through from the weak AE resin, g) loading the composition comprising the anti-c-met antibody on a strong CE resin (e.g., SP Sepharose Flast Flow, POROS HS 50, or Toyopearl SP 550c) and h) eluting the anti-c-met antibody from the strong CE resin. The steps of the method of purifying the anti-c-met antibody can be completed in any order. In some embodiments, the steps are done sequentially. In some embodiments, the anti-c-met antibody is produced in E. coli. In some embodiments, the anti-c-met antibody is onartuzumab.

In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE) and recovering the anti-c-met antibody in the flow-through from the weak AE resin, d) loading the composition comprising the anti-c-met antibody on a strong CE resin (e.g., SP Sepharose Flast Flow, POROS HS 50, or Toyopearl SP 550c) and e) eluting the anti-c-met antibody from the strong CE resin, and wherein the HCP (e.g., average HCP) is reduced to less than about 70 ng/mg. In some embodiments, the HCP (e.g., average HCP) is reduced to less than or equal to about any of 60 ng/mg, 55 ng/mg, 50 ng/mg, 45 ng/mg, 40 ng/mg, 35 ng/mg, or 30 ng/mg. In some embodiments, the HCP (e.g., average HCP) is reduced to between about 30 ng/mg and about 50 ng/mg or between about 35 ng/mg and about 45 ng/mg. In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE), f) recovering the anti-c-met antibody in the flow-through from the weak AE resin, g) loading the composition comprising the anti-c-met antibody on a strong CE resin (e.g., SP Sepharose Flast Flow, POROS HS 50, or Toyopearl SP 550c), and e) eluting the anti-c-met antibody from the strong CE resin, and wherein the HCP (e.g., average HCP) is reduced by greater than about 85%, 80%, 75%, 70%, 65%, or 60% compared to the same method of purification in the absence of the flocculation step, Prosep vA as the protein A affinity chromatography resin, and/or a weak CE resin (e.g., CM Sepharose). In some embodiments, the steps are done sequentially. In some embodiments, the anti-c-met antibody is produced in E. coli. In some embodiments, the HCP and/or average HCP is ECP and/or average ECP. In some embodiments, the anti-c-met antibody is onartuzumab.

In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™resin), d) eluting the anti-c-met antibody from the protein A affinity resin, e) loading the composition comprising the anti-c-met antibody on a strong CE resin (e.g., SP Sepharose Flast Flow, POROS HS 50, or Toyopearl SP 550c), f) eluting the anti-c-met antibody from the strong CE resin, g) loading a composition comprising the anti-c-met antibody on a weak AE resin (e.g., DEAE Sepharose Fast Flow or Capto DEAE), and h) recovering the anti-c-met antibody in the flow-through from the weak AE resin, and wherein the HCP (e.g., average HCP) is reduced to less than about 70 ng/mg. In some embodiments, the HCP (e.g., average HCP) is reduced to less than or equal to about any of 60 ng/mg, 55 ng/mg, 50 ng/mg, 45 ng/mg, 40 ng/mg, 35 ng/mg, or 30 ng/mg. In some embodiments, the HCP (e.g., average HCP) is reduced to between about 30 ng/mg and about 50 ng/mg or between about 35 ng/mg and about 45 ng/mg. In some embodiments, the method comprises a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading the composition comprising the anti-c-met antibody on a strong CE resin (e.g., SP Sepharose Flast Flow, POROS HS 50, or Toyopearl SP 550c), f) eluting the anti-c-met antibody from the strong CE resin, g) loading a composition comprising the anti-c-met antibody on a weak AE resin (e.g., DEAE Sepharose Fast Flow or Capto DEAE), and h) recovering the anti-c-met antibody in the flow-through from the weak AE resin, and wherein the HCP (e.g., average HCP) is reduced by greater than about 85%, 80%, 75%, 70%, 65%, or 60% compared to the same method of purification in the absence of the flocculation step, Prosep vA as the protein A affinity chromatography resin, and/or a weak CE resin (e.g., CM Sepharose). In some embodiments, the steps are done sequentially. In some embodiments, the anti-c-met antibody is produced in E. coli. In some embodiments, the HCP and/or average HCP is ECP and/or average ECP. In some embodiments, the anti-c-met antibody is onartuzumab.

In some embodiments of any of the methods of purification described, the method further comprises loading a composition comprising the anti-c-met antibody on a strong AE resin and eluting the anti-c-met antibody. In some embodiments, the anti-c-met antibody is produced in E. coli. In some embodiments, the anti-c-met antibody is onartuzumab.

Strong AE exchange resins generally contain a quaternary ammonium ion. Examples of strong AE resins are known in the art and include, but are not limited to, Mini Q PC 3.2/3, Mini Q 4.6/50 PE, Mono Q 5/50 GL, Mono Q PC 1.6/5, RESOURCE Q, HiTrap Q HP, HiTrap Q FF, HiPrep SP FF, Q Sepharose Fast Flow, Capto Q, HiTrap Q XL, POROS HQ 50, Toyopearl SuperQ-650C, Toyopearl QAE-550C, Toyopearl Q-600CAR, Toyopeawrl GigaCap Q-650M, Toyopearl SuperQ-650M, Toyopearl Super Q-6505, TSKgel SuperQ-5PW 30, TSKgel SuperQ-5PW 20, and/or Fractogel TMAE. In some embodiments, the strong AE resin is Q Sepharose Fast Flow (—O—CH₂CHOHCH₂OCH₂CHOHCH₂N⁺(CH₃)₃ surface functionality attached to a highly cross-linked agarose support matrix). In some embodiments, the strong AE resin is Capto Q. In some embodiments, the strong AE resin is Q Sepharose Fast Flow.

In some embodiments, the flow rate for the strong AE chromatography is between about any of 50-500 cm/hr, 50-250 cm/hr, and/or 250-500 cm/hour. In some embodiments, the flow rate is about any of 105 cm/hour, 125 cm/hour, 135 cm/hour, 145 cm/hour, 155 cm/hour, 165 cm/hour, 185 cm/hr, and/or 250 cm/hr.

In some embodiments, the conductivity for the strong AE chromatography is less than about 1.9 mS/cm at about pH 8.9-9.0 and/or less than about 2.4 mS/cm at pH 9.0 or greater. In some embodiments, the conductivity is between about 1.4 mS/cm and about 1.9 mS/cm at about pH 8.9-pH 9.0 or between about 1.4 mS/cm and about 1.9 mS/cm at about pH 8.9-pH 9.5.

The strong AE resin can be equilibrated with a pre-equilibration buffer followed by an equilibration buffer, and the unpurified or partially purified anti-c-met antibodies comprising various impurities (e.g., harvested cell proteins (e.g., ECP)) can then be loaded onto the equilibrated resin. As the anti-c-met antibodies flow through the resin, the anti-c-met antibodies and various impurities are adsorbed to the immobilized strong AE resin. The wash buffers can be used to remove some impurities, such as host cell impurities, but not anti-c-met antibodies. In some embodiments, the equilibration buffer is utilized as the wash buffer. The anti-c-met antibodies are eluted from the resin with the elution buffer.

The pre-equilibration buffer for the strong AE chromatography may comprise Tris and a salt. Examples of salt useful in the pre-equilibration buffer include, but are not limited to, potassium chloride, sodium chloride, magnesium sulfate, sodium sulfate, sodium acetate, and/or sodium citrate. In some embodiments, the salt is potassium chloride. In some embodiments, the salt is sodium chloride. In some embodiments, the concentration of Tris in the equilibration buffer is between about 0.01 M and about 0.1 M. For example, in some embodiments, the concentration of Tris is about any of 0.01 M, 0.025 M, 0.05 M, 0.075 M, or 0.1 M. In some embodiments, the concentration of salt is between about 0.1 M and about 1.0 M. For example, in some embodiments, the concentration of salt is about any of 0.1 M, 0.25 M, 0.5 M, 0.75 M, or 1.0 M. In some embodiments, the pH of the pre-equilibration buffer is about any of 8.7, 8.8, 8.9, 9.0, 9.1, or 9.2.

The equilibration buffer for the strong AE chromatography may comprise Tris and a salt. Examples of salt useful in the equilibration buffer include, but are not limited to, potassium chloride, sodium chloride, magnesium sulfate, sodium sulfate, sodium acetate, and/or sodium citrate. In some embodiments, the salt is potassium chloride. In some embodiments, the salt is sodium chloride. In some embodiments, the concentration of Tris in the equilibration buffer is between about 0.01 M and about 0.1 M. For example, in some embodiments, the concentration of Tris is about any of 0.01 M, 0.025 M, 0.05 M, 0.075 M, or 0.1 M. In some embodiments, the concentration of salt is between about 0.01 M and about 0.1 M. For example, in some embodiments, the concentration of salt is about any of 0.01M, 0.025 M, 0.05 M, 0.075 M, or 0.1M. In some embodiments, the pH of the equilibration buffer is about any of 8.7, 8.8, 8.9, 9.0, 9.1, or 9.2.

The wash buffer for the strong AE chromatography may comprise Tris and a salt. Examples of salt useful in the wash buffer include, but are not limited to, potassium chloride, sodium chloride, magnesium sulfate, sodium sulfate, sodium acetate, and/or sodium citrate. In some embodiments, the salt is potassium chloride. In some embodiments, the salt is sodium chloride. In some embodiments, the concentration of Tris in the equilibration buffer is between about 0.01 M and about 0.1 M. For example, in some embodiments, the concentration of Tris is about any of 0.01 M, 0.025 M, 0.05 M, 0.075 M, or 0.1 M. In some embodiments, the concentration of salt is between about 0.01 M and 0.1 M. For example, in some embodiments, the concentration of salt is about any of 0.01 M, 0.025 M, 0.05 M, 0.075 M, or 0.1 M. In some embodiments, the pH of the wash buffer is about any of 8.7, 8.8, 8.9, 9.0, 9.1, or 9.2.

The elution buffer for the strong AE chromatography may comprise Tris and a salt. Examples of salt useful in the pre-equilibration buffer include, but are not limited to, potassium chloride, sodium chloride, magnesium sulfate, sodium sulfate, sodium acetate, and/or sodium citrate. In some embodiments, the salt is potassium chloride. In some embodiments, the salt is sodium chloride. In some embodiments, the concentration of Tris in the equilibration buffer is between about 0.01 M and about 0.1 M. For example, in some embodiments, the concentration of Tris is about any of 0.01 M, 0.025 M, 0.05 M, 0.075 M, or 0.1 M. In some embodiments, the concentration of salt is between about 0.015 M and 0.15 M. For example, in some embodiments, the concentration of salt is about any of 0.015 M, 0.045 M, 0.075 M, 0.095 M, or 0.115 M. In some embodiments, the pH of the wash buffer is about any of 8.7, 8.8, 8.9, 9.0, 9.1, or 9.2.

In some embodiments, the method further comprises a flocculation step such as described above. In some embodiments, the method further comprises centrifugation. In some embodiments, the method further comprises protein A affinity chromatography as described above. In some embodiments, the method further comprises one or more additional ion-exchange chromatography steps such as any of those described herein. In some embodiments, the method further comprises ultrafiltration and/or diafiltration. In some embodiments, the method comprises a) the flocculation step followed by b) centrifugation step followed by c) affinity chromatography (e.g., protein A affinity chromatography) followed by d) weak AE chromatography followed by e) strong CE chromatography followed by f) strong AE chromatography. For example, in some embodiments, methods of purifying a composition comprising an anti-c-met antibody comprising a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE), f) recovering the anti-c-met antibody in the flow-through from the weak AE resin, g) loading the composition comprising the anti-c-met antibody on a strong CE resin (e.g., SP Sepharose Flast Flow, POROS HS 50, or Toyopearl SP 550c), h) eluting the anti-c-met antibody from the strong CE resin, i) loading the composition comprising the anti-c-met antibody on a strong AE resin (e.g., Q Sepharose Fast Flow, Capto Q, or POROS HQ 50), and j) eluting the anti-c-met antibody from the strong AE resin. The steps of the method of purifying the anti-c-met antibody can be completed in any order. In some embodiments, the steps are done sequentially. In some embodiments, the anti-c-met antibody is produced in E. coli. In some embodiments, the anti-c-met antibody is onartuzumab.

In some embodiments, the method comprising a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE), f) recovering the anti-c-met antibody in the flow-through from the weak AE resin, g) loading the composition comprising the anti-c-met antibody on a strong CE resin (e.g., SP Sepharose Flast Flow, POROS HS 50, or Toyopearl SP 550c) h) eluting the anti-c-met antibody from the strong CE resin, i) loading the composition comprising the anti-c-met antibody on a strong AE resin (e.g., Q Sepharose Fast Flow, Capto Q, or POROS HQ 50) and j) eluting the anti-c-met antibody from the strong AE resin, and wherein the HCP (e.g., average HCP) is reduced to less than about 50 ng/mg. In some embodiments, the HCP (e.g., average HCP) is reduced to less than or equal to about any of 34 ng/mg, 30 ng/mg, 25 ng/mg, 20 ng/mg, 15 ng/mg, 14 ng/mg, 13 ng/mg, 12 ng/mg, 11 ng/mg, or 10 ng/mg. In some embodiments, the HCP (e.g., average HCP) is reduced to between about 1 ng/mg and about 15 ng/mg or between about 5 ng/mg and about 15 ng/mg. In some embodiments, the method comprising a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE), f) recovering the anti-c-met antibody in the flow-through from the weak AE resin, g) loading the composition comprising the anti-c-met antibody on a strong CE resin (e.g., SP Sepharose Flast Flow, POROS HS 50, or Toyopearl SP 550c) h) eluting the anti-c-met antibody from the strong CE resin, i) loading the composition comprising the anti-c-met antibody on a strong AE resin (e.g., Q Sepharose Fast Flow, Capto Q, or POROS HQ 50) and j) eluting the anti-c-met antibody from the strong AE resin, and wherein the HCP (e.g., average HCP) is reduced by greater than about 55%, 50%, 45%, 40%, 35%, or 30% compared to the same method of purification in the absence of the flocculation step, Prosep vA as the protein A affinity chromatography resin, and/or a weak CE resin (e.g., CM Sepharose). In some embodiments, the steps are done sequentially. In some embodiments, the anti-c-met antibody is produced in E. coli. In some embodiments, the HCP and/or average HCP is ECP and/or average ECP. In some embodiments, the anti-c-met antibody is onartuzumab.

In some embodiments of any of the methods described herein, the method further comprises ultrafiltration and/or diafiltration. In some embodiments, the method comprises a) the flocculation step followed by b) centrifugation step followed by c) affinity chromatography (e.g., protein A affinity chromatography) followed by d) weak AE chromatography followed by e) strong CE chromatography followed by f) strong AE chromatography followed by g) ultrafiltration and/or diafiltration. For example, in some embodiments, methods of purifying a composition comprising an anti-c-met antibody comprising a) keeping the composition comprising the anti-c-met antibody at a temperature of greater than 28° C. and a pH between about pH 6 and about pH 8 for more than 6 hours, b) centrifuging the composition comprising the anti-c-met antibody, c) loading the composition comprising the anti-c-met antibody on a protein A affinity resin (e.g., MabSelect SuRe™ resin), d) eluting the anti-c-met antibody from the protein A affinity resin, e) loading a composition comprising the anti-c-met antibody on a weak AE resin (e.g., DEAE Sepharose Fast Flow or Capto DEAE), f) recovering the anti-c-met antibody in the flow-through from the weak AE resin, g) loading the composition comprising the anti-c-met antibody on a strong CE resin (e.g., SP Sepharose Flast Flow, POROS HS 50, or Toyopearl SP 550c) h) eluting the anti-c-met antibody from the strong CE resin, i) loading the composition comprising the anti-c-met antibody on a strong AE resin (e.g., Q Sepharose Fast Flow, Capto Q, or POROS HQ 50), j) eluting the anti-c-met antibody from the strong AE resin, and k) subjecting the elutant from the strong AE resin comprising the anti-c-met antibody to ultrafiltration (e.g., 10 KDa regenerated cellulose ultrafiltration membrane) and/or diafiltration. The steps of the method of purifying the anti-c-met antibody can be completed in any order. In some embodiments, the steps are done sequentially. In some embodiments, the anti-c-met antibody is produced in E. coli.

In some embodiments of any of the methods of purifying, the HCP present in the composition comprising an anti-c-met antibody is less than or equal to about 50 ng/mg. In some embodiments of any of the methods of purifying, the average HCP present in a lot (e.g., batch) of the composition comprising an anti-c-met antibody is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the DNA levels in the composition comprising an anti-c-met antibody are less than or equal to about 0.3 pg/mg. In some embodiments of any of the methods of purifying, the average DNA levels in a lot (e.g., batch) of the composition comprising an anti-c-met antibody are less than or equal to about 0.3 pg/mg. In some embodiments, the DNA levels and/or average DNA levels are less than or equal to about any of 0.3 pg/mg, 0.25 pg/mg, 0.2 pg/mg, 0.15 pg/mg, or 0.1 pg/mg. In some embodiments, the DNA levels and/or average DNA levels are between about any of 0.001 pg/mg and 0.3 pg/mg, 0.001 pg/mg and 0.2 pg/mg, 0.001 pg/mg and 0.1 pg/mg, 0.01 pg/mg and 0.3 pg/mg, 0.01 pg/mg and 0.2 pg/mg, or 0.01 pg/mg and 0.1 pg/mg. In some embodiments, the DNA levels and/or average DNA levels are about any of 0.3, 0.25, 0.2, 0.15, or 0.1 pg/mg. In some embodiments, DNA levels are determined by PCR. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the leached protein A (LpA) in the composition comprising an anti-c-met antibody is less than or equal to about 2 ng/mg. In some embodiments of any of the methods of purifying, the average LpA in a lot (e.g., batch) of the composition comprising an anti-c-met antibody is less than or equal to about 2 ng/mg. In some embodiments, the LpA and/or average LpA is between about any of 0.001 ng/mg and 2 ng/mg, 0.01 ng/mg and 2 ng/mg, 0.1 ng/mg and 2 ng/mg, or 1 ng/mg and 2 ng/mg. In some embodiments, the LpA and/or average LpA is about any of 1, 1.25, 1.5, 1.75, or 2 ng/mg. In some embodiments, percentage of LpA is determined by Leached protein A ligand assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the Limulus Amebocyte Lysate (LAL) in the composition comprising an anti-c-met antibody is less than or equal to about 0.01 EU/mg. In some embodiments of any of the methods of purifying, the average LAL in a lot (e.g., batch) of the composition comprising an anti-c-met antibody is less than or equal to about 0.01 EU/mg. In some embodiments, the LAL and/or average LAL is less than or equal to about any of 0.007 EU/mg, 0.006 EU/mg, 0.005 EU/mg, 0.002 EU/mg, or 0.001 EU/mg. In some embodiments, the LAL and/or average LAL is between about any of 0.0001 EU/mg and 0.01 EU/mg, 0.0001 EU/mg and 0.007 EU/mg, 0.0001 EU/mg and 0.006 EU/mg, or 0.0001 EU/mg and 0.005 EU/mg. In some embodiments, the LAL and/or average LAL is about any of 0.01, 0.007, 0.006, 0.005, 0.004, 0.003, or 0.002 EU/mg. In some embodiments, percentage of LAL is determined by LAL assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the percentage of aggregates in the composition comprising an anti-c-met antibody is less than or equal to about 0.3%. In some embodiments of any of the methods of purifying, the average percentage of aggregates in a lot (e.g., batch) of the composition comprising an anti-c-met antibody is less than or equal to about 0.3%. In some embodiments, the percentage of aggregates and/or average percentage of aggregates is less than or equal to about any of 0.2% or 0.1%. In some embodiments, the percentage of aggregates and/or average percentage of aggregates is between about any of 0.001% and 0.3%, 0.01% and 0.3%, 0.001% and 0.2%, or 0.01% and 0.2%. In some embodiments, the percentage of aggregates and/or average percentage of aggregates is about any of 0.3%, 0.25%, 0.2%, 0.15%, or 0.1%. In some embodiments, percentage of aggregates is determined by size exclusion chromatography (SEC) assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the percentage of monomer in the composition comprising an anti-c-met antibody is greater than or equal to about 99.5%. In some embodiments of any of the methods of purifying, the average percentage monomer in a lot (e.g., batch) of the composition comprising an anti-c-met antibody is greater than or equal to about 99.5%. In some embodiments, the percentage of monomer and/or average percentage of monomer is greater than or equal to about any of 99.6%, 99.7%, 99.8%, or 99.9%. In some embodiments, the percentage of monomer and/or average percentage of monomer is between about any of 99.5% and 99.999%, 99.5% and 99.99%, 99.6% and 99.999%, 99.6% and 99.99%, 99.7% and 99.999%, 99.7% and 99.99%, 99.8% and 99.999%, 99.8% and 99.99%, or 99.9% and 99.999%, 99.9% and 99.99%. In some embodiments, the percentage of monomer and/or average percentage of monomer is about any of 99.5%, 99.6%, 99.7%, 99.8%, or 99.9%. In some embodiments, percentage of monomer is determined by SEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the percentage of fragments in the composition comprising an anti-c-met antibody is less than or equal to about 0.3%. In some embodiments of any of the methods of purifying, the average percentage of fragments in a lot (e.g., batch) of the composition comprising an anti-c-met antibody is less than or equal to about 0.3%. In some embodiments, the percentage of fragments and/or average percentage of fragments is less than or equal to about any of 0.2% or 0.1%. In some embodiments, the percentage of fragments and/or average percentage of fragments is between about any of 0.001% and 0.3%, 0.01% and 0.3%, 0.001% and 0.2%, or 0.01% and 0.2%. In some embodiments, the percentage of fragments and/or average percentage of fragments is about any of 0.3%, 0.25%, 0.2%, 0.15%, 0.1%, or 0%. In some embodiments, fragments are not detectable. In some embodiments, percentage of fragments is determined by SEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the percentage of acidic variants in the composition comprising an anti-c-met antibody is less than or equal to about 20%. In some embodiments of any of the methods of purifying, the average percentage of acidic variants in a lot (e.g., batch) of the composition comprising an anti-c-met antibody is less than or equal to about 20%. In some embodiments, the percentage of acidic variants and/or average percentage of acidic variants is less than or equal to about any of 20%, 18.5%, 17.5%, 15%, 12.5%. In some embodiments, the percentage of acidic variants and/or average percentage of acidic variants is between about any of 1% and 20%, 5% and 20%, or 10% and 20%. In some embodiments, the percentage of acidic variants and/or average percentage of acidic variants is about any of 20%, 18.5%, 17.5%, 15%, or 12.5%. In some embodiments, percentage of acidic variants is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the percentage of main peak in the composition comprising an anti-c-met antibody is greater than or equal to about 75%. In some embodiments of any of the methods of purifying, the average percentage of main peak in a lot (e.g., batch) of the composition comprising an anti-c-met antibody is greater than or equal to about 75%. In some embodiments, the percentage of main peak and/or average percentage of main peak greater than or equal to about any of 77.5%, 80%, 82.5%, or 85%. In some embodiments, the percentage of main peak and/or average percentage of main peak is between about any of 75% and 95%, 77.5% and 95%, 80% and 95%, 82.5% and 95%, or 85% and 95%. In some embodiments, the percentage of main peak and/or average percentage of main peak is about any of 75%, 77.5%, 80%, 82.5%, or 85%. In some embodiments, percentage of main peak is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the methods of purifying, the percentage of basic variants in the composition comprising an anti-c-met antibody is less than or equal to about 2.0%. In some embodiments of any of the methods of purifying, the average percentage of basic variants in a lot (e.g., batch) of the composition comprising an anti-c-met antibody is less than or equal to about 2.0%. In some embodiments, the percentage of basic variants and/or average percentage of basic variants is less than or equal to about any of 1.5%, 1.25%, 1.1%, or 1%. In some embodiments, the percentage of basic variants and/or average percentage of basic variants is between about any of 0.001% and 2%, 0.01% and 2%, 0.001% and 1.5%, or 0.01% and 1.5%, 0.001% and 1.0%, or 0.01% and 1.0%. In some embodiments, the percentage of basic variants and/or average percentage of basic variants is about any of 2%, 1.5%, 1.25%, 1.1%, or 1%. In some embodiments, percentage of basic variants is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

Further provided herein are purified anti-c-met antibodies and compositions comprising purified anti-c-met antibodies. In some embodiments, the purified anti-c-met antibodies are purified by any of the methods of purification described herein. In some embodiments, the purified anti-c-met antibodies are obtainable by any of the methods of purification described herein. In some embodiments, the HCP present in the composition comprising purified anti-c-met antibodies purified and/or obtainable by any of the methods of purification described herein is less than or equal to about 50 ng/mg. In some embodiments, the average HCP present in a lot (e.g., batch) of the composition comprising purified anti-c-met antibodies purified and/or obtainable by any of the methods of purification described herein is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

Provided herein are compositions comprising an anti-c-met antibody, wherein HCP present in the composition is less than or equal to about 50 ng/mg. Further provided herein are lots (e.g., batches) of a composition comprising an anti-c-met antibody, wherein the average HCP present in the lot (e.g., batch) is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the DNA levels in the composition comprising an anti-c-met antibody are less than or equal to about 0.3 pg/mg. In some embodiments of any of the compositions, the average DNA levels in a lot (e.g., batch) of the composition comprising an anti-c-met antibody are less than or equal to about 0.3 pg/mg. In some embodiments, the DNA levels and/or average DNA levels are less than or equal to about any of 0.3 pg/mg, 0.25 pg/mg, 0.2 pg/mg, 0.15 pg/mg, or 0.1 pg/mg. In some embodiments, the DNA levels and/or average DNA levels are between about any of 0.001 pg/mg and 0.3 pg/mg, 0.001 pg/mg and 0.2 pg/mg, 0.001 pg/mg and 0.1 pg/mg, 0.01 pg/mg and 0.3 pg/mg, 0.01 pg/mg and 0.2 pg/mg, or 0.01 pg/mg and 0.1 pg/mg. In some embodiments, the DNA levels and/or average DNA levels are about any of 0.3, 0.25, 0.2, 0.15, or 0.1 pg/mg. In some embodiments, DNA levels are determined by PCR. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the leached protein A (LpA) in the composition comprising an anti-c-met antibody is less than or equal to about 2 ng/mg. In some embodiments of any of the compositions, the average LpA in a lot (e.g., batch) of the composition comprising an anti-c-met antibody is less than or equal to about 2 ng/mg. In some embodiments, the LpA and/or average LpA is between about any of 0.001 ng/mg and 2 ng/mg, 0.01 ng/mg and 2 ng/mg, 0.1 ng/mg and 2 ng/mg, or 1 ng/mg and 2 ng/mg. In some embodiments, the LpA and/or average LpA is about any of 1, 1.25, 1.5, 1.75, or 2 ng/mg. In some embodiments, percentage of LpA is determined by Leached protein A ligand assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the Limulus Amebocyte Lysate (LAL) in the composition comprising an anti-c-met antibody is less than or equal to about 0.01 EU/mg. In some embodiments of any of the compositions, the average LAL in a lot (e.g., batch) of the composition comprising an anti-c-met antibody is less than or equal to about 0.01 EU/mg. In some embodiments, the LAL and/or average LAL is less than or equal to about any of 0.007 EU/mg, 0.006 EU/mg, 0.005 EU/mg, 0.002 EU/mg, or 0.001 EU/mg. In some embodiments, the LAL and/or average LAL is between about any of 0.0001 EU/mg and 0.01 EU/mg, 0.0001 EU/mg and 0.007 EU/mg, 0.0001 EU/mg and 0.006 EU/mg, or 0.0001 EU/mg and 0.005 EU/mg. In some embodiments, the LAL and/or average LAL is about any of 0.01, 0.007, 0.006, 0.005, 0.004, 0.003, or 0.002 EU/mg. In some embodiments, percentage of LAL is determined by LAL assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the percentage of aggregates in the composition comprising an anti-c-met antibody is less than or equal to about 0.3%. In some embodiments of any of the compositions, the average percentage of aggregates in a lot (e.g., batch) of the composition comprising an anti-c-met antibody is less than or equal to about 0.3%. In addition, provided herein are compositions comprising an anti-c-met antibody, wherein percentage of aggregates present in the composition is less than or equal to about 0.3%. Further provided herein are lots (e.g., batches) of a composition comprising an anti-c-met antibody, wherein the average percentage of aggregates present in the composition is less than or equal to about 0.3%. In some embodiments, the percentage of aggregates and/or average percentage of aggregates is less than or equal to about any of 0.2% or 0.1%. In some embodiments, the percentage of aggregates and/or average percentage of aggregates is between about any of 0.001% and 0.3%, 0.01% and 0.3%, 0.001% and 0.2%, or 0.01% and 0.2%. In some embodiments, the percentage of aggregates and/or average percentage of aggregates is about any of 0.3%, 0.25%, 0.2%, 0.15%, or 0.1%. In some embodiments, percentage of aggregates is determined by size exclusion chromatography (SEC) assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the percentage of monomer in the composition comprising an anti-c-met antibody is greater than or equal to about 99.5%. In some embodiments of any of compositions, the average percentage monomer in a lot (e.g., batch) of the composition comprising an anti-c-met antibody is greater than or equal to about 99.5%. In addition, provided herein are compositions comprising an anti-c-met antibody, wherein the percentage of monomer present in the composition is greater than or equal to about 99.5%. Further provided herein are lots (e.g., batches) of a composition comprising an anti-c-met antibody, wherein the average percentage of monomer present in the composition is greater than or equal to about 0.3%. In some embodiments, the percentage of monomer and/or average percentage of monomer is greater than or equal to about any of 99.6%, 99.7%, 99.8%, or 99.9%. In some embodiments, the percentage of monomer and/or average percentage of monomer is between about any of 99.5% and 99.999%, 99.5% and 99.99%, 99.6% and 99.999%, 99.6% and 99.99%, 99.7% and 99.999%, 99.7% and 99.99%, 99.8% and 99.999%, 99.8% and 99.99%, or 99.9% and 99.999%, 99.9% and 99.99%. In some embodiments, the percentage of monomer and/or average percentage of monomer is about any of 99.5%, 99.6%, 99.7%, 99.8%, or 99.9%. In some embodiments, percentage of monomer is determined by SEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the percentage of fragments in the composition comprising an anti-c-met antibody is less than or equal to about 0.3%. In some embodiments of any of the compositions, the average percentage of fragments in a lot (e.g., batch) of the composition comprising an anti-c-met antibody is less than or equal to about 0.3%. In addition, provided herein are compositions comprising an anti-c-met antibody, wherein percentage of fragments present in the composition is less than or equal to about 0.3%. Further provided herein are lots (e.g., batches) of a composition comprising an anti-c-met antibody, wherein the average percentage of fragments present in the composition is less than or equal to about 0.3%. In some embodiments, the percentage of fragments and/or average percentage of fragments is less than or equal to about any of 0.2% or 0.1%. In some embodiments, the percentage of fragments and/or average percentage of fragments is between about any of 0.001% and 0.3%, 0.01% and 0.3%, 0.001% and 0.2%, or 0.01% and 0.2%. In some embodiments, the percentage of fragments and/or average percentage of fragments is about any of 0.3%, 0.25%, 0.2%, 0.15%, 0.1%, or 0%. In some embodiments, fragments are not detectable. In some embodiments, percentage of fragments is determined by SEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the percentage of acidic variants in the composition comprising an anti-c-met antibody is less than or equal to about 20%. In some embodiments of any of the compositions, the average percentage of acidic variants in a lot (e.g., batch) of the composition comprising an anti-c-met antibody is less than or equal to about 20%. In addition, provided herein are compositions comprising an anti-c-met antibody, wherein percentage of acidic variants present in the composition is less than or equal to about 20%. Further provided herein are lots (e.g., batches) of a composition comprising an anti-c-met antibody, wherein the average acidic variants present in the composition is less than or equal to about 20%. In some embodiments, the percentage of acidic variants and/or average percentage of acidic variants is less than or equal to about any of 20%, 18.5%, 17.5%, 15%, 12.5%. In some embodiments, the percentage of acidic variants and/or average percentage of acidic variants is between about any of 1% and 20%, 5% and 20%, or 10% and 20%. In some embodiments, the percentage of acidic variants and/or average percentage of acidic variants is about any of 20%, 18.5%, 17.5%, 15%, or 12.5%. In some embodiments, percentage of acidic variants is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the percentage of main peak in the composition comprising an anti-c-met antibody is greater than or equal to about 75%. In some embodiments of any of the compositions, the average percentage of main peak in a lot (e.g., batch) of the composition comprising an anti-c-met antibody is greater than or equal to about 75%. In addition, provided herein are compositions comprising an anti-c-met antibody, wherein percentage of main peak present in the composition is greater than or equal to about 75%. Further provided herein are lots (e.g., batches) of a composition comprising an anti-c-met antibody, wherein the average percentage of main peak present in the composition is greater than or equal to about 75%. In some embodiments, the percentage of main peak and/or average percentage of main peak greater than or equal to about any of 77.5%, 80%, 82.5%, or 85%. In some embodiments, the percentage of main peak and/or average percentage of main peak is between about any of 75% and 95%, 77.5% and 95%, 80% and 95%, 82.5% and 95%, or 85% and 95%. In some embodiments, the percentage of main peak and/or average percentage of main peak is about any of 75%, 77.5%, 80%, 82.5%, or 85%. In some embodiments, percentage of main peak is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the percentage of basic variants in the composition comprising an anti-c-met antibody is less than or equal to about 2.0%. In some embodiments of any of the compositions, the average percentage of basic variants in a lot (e.g., batch) of the composition comprising an anti-c-met antibody is less than or equal to about 2.0%. In addition, provided herein are compositions comprising an anti-c-met antibody, wherein percentage of basic variants present in the composition is less than or equal to about 2.0%. Further provided herein are lots (e.g., batches) of a composition comprising an anti-c-met antibody, wherein the average percentage of basic variants present in the composition is less than or equal to about 2.0%. In some embodiments, the percentage of basic variants and/or average percentage of basic variants is less than or equal to about any of 1.5%, 1.25%, 1.1%, or 1%. In some embodiments, the percentage of basic variants and/or average percentage of basic variants is between about any of 0.001% and 2%, 0.01% and 2%, 0.001% and 1.5%, or 0.01% and 1.5%, 0.001% and 1.0%, or 0.01% and 1.0%. In some embodiments, the percentage of basic variants and/or average percentage of basic variants is about any of 2%, 1.5%, 1.25%, 1.1%, or 1%. In some embodiments, percentage of basic variants is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the compositions, the anti-c-met antibody (e.g., onartuzumab) concentration in the composition comprising an anti-c-met antibody is greater than or equal to about any of 0.5 mg/mL, 1 mg/mL, 1.5 mg/mL, or 2 mg/mL. In some embodiments of any of the compositions, the anti-c-met antibody (e.g., onartuzumab) concentration in a lot (e.g., batch) of the composition comprising an anti-c-met antibody is less than or equal to about any of 0.5 mg/mL, 1 mg/mL, 1.5 mg/mL, or 2 mg/mL.

Levels of HCP (e.g., ECP) can be measured by methods known in the art. For example, a multiproduct sandwich ELISA for E. Coli Proteins may be used to quantitate the levels of ECP. Affinity-purified goat anti-whole ECP antibodies are immobilized on microtiter plate wells. Dilutions of the pool samples are incubated in the wells, followed by an incubation with affinity-purified goat anti-whole ECP conjugated to horseradish peroxidase. The horseradish peroxidase enzymatic activity is detected with o-phenylenediamine dihydrochloride. The ECP is quantitated by reading absorbance at 490 nm in a microtiter plate reader. A 4-parameter computer curve fitting program is used to generate the standard curve, and automatically calculate the sample concentration. Prior to the assay, samples are diluted with assay diluent. Serial 2-fold dilutions in assay diluent may be performed so that the absorbance reading falls within the range of the standard curve. The assay range for the ELISA is typically 1.56 ng/mL to 100 ng/mL.

In addition, the DNA levels can be measured by methods known in the art including, but not limited to, PCR or rtPCT as described in the Examples. LpA levels can be measured by methods known in the art including, but not limited to, ELISA as described in the Examples. The kinetic chromogenic method LAL assay can be used to measure bacterial endotoxins, which is described herein as Limulus Amebocyte Lysate (LAL) as described in the Examples. Percentage of monomers, aggregate, and fragments can be measured by methods known in the art including, but not limited to, size exclusion chromatography as described in the Examples. Percentage main peak, acidic variant, and basic variant can be measured by methods known in the art including, but not limited to, cation-exchange chromatography as described in the Examples.

### III. RECOMBINANT METHODS

The anti-c-met antibody for use in the purified anti-c-met antibody compositions and/or methods of purification described herein may be produced recombinant methods and compositions, e.g., as described in U.S. Pat. No. 4,816,567. In one embodiment, isolated nucleic acid encoding an antibody is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In a further embodiment, a host cell comprises (e.g., has been transformed with): (1) a vector comprising a nucleic acid that encodes an amino acid sequence comprising the VL of the antibody and an amino acid sequence comprising the VH of the antibody and an amino acid sequence comprising the Fc region, or (2) a first vector comprising a nucleic acid that encodes an amino acid sequence comprising the VL of the antibody and a second vector comprising a nucleic acid that encodes an amino acid sequence comprising the VH of the antibody and a third vector comprising a nucleic acid that encodes an amino acid sequence comprising the Fc region. Production of a one-armed antibody is described, e.g., in WO2005/063816.

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523, WO/05/063816. (See also Charlton, Methods in Molecular Biology, Vol. 248 (B.K.C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

In one embodiment, the host cell is prokaryotic, e.g. E. coli. In one embodiment, a method of making an antibody is provided, wherein the method comprises culturing an E. coli host cell comprising a nucleic acid encoding the anti-c-met antibody under conditions suitable for expression of the anti-c-met antibody, and recovering the anti-c-met antibody from the E. coli host cell (or host cell culture medium) by a method described above. In some embodiments, the anti-c-met antibody is onartuzumab.

In one embodiment, the host cell is eukaryotic, e.g. a Chinese Hamster Ovary (CHO) cell or lymphoid cell (e.g., Y0, NS0, Sp20 cell). In one embodiment, a method of making an antibody is provided, wherein the method comprises culturing a host cell comprising a nucleic acid encoding the anti-c-met antibody under conditions suitable for expression of the anti-c-met antibody, and recovering the anti-c-met antibody from the host cell (or host cell culture medium) by a method described above.

For recombinant production of an antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

### IV. ANTI-C-MET ANTIBODIES

Provided herein are compositions comprising purified anti-c-met antibodies and/or anti-c-met antibodies for use in the methods of purification described herein. Useful anti-c-met antibodies include antibodies that bind with sufficient affinity and specificity to c-met and can reduce or inhibit one or more c-met activities. Anti-c-met antibodies of the purified anti-c-met antibody compositions and/or for use in the methods of purification can be used to modulate one or more aspects of HGF/c-met-associated effects, including but not limited to c-met activation, downstream molecular signaling (e.g., mitogen activated protein kinase (MAPK) phosphorylation), cell proliferation, cell migration, cell survival, cell morphogenesis and angiogenesis. These effects can be modulated by any biologically relevant mechanism, including disruption of ligand (e.g., HGF) binding to c-met, c-met phosphorylation and/or c-met multimerization. In some embodiments, the anti-c-met antibody is an antagonist anti-c-met antibody. In some embodiments, the anti-c-met antibody interferes with diseases or conditions wherein c-met/HGF activity is involved.

In some embodiments of any of the purified anti-c-met antibody compositions and/or methods of purification described herein, the anti-c-met antibody is an antagonist anti-c-met antibody. In some embodiments, the anti-c-met antibody is an anti-c-met antibody fragment. In some embodiments, the anti-c-met antibody is an IgG1 antibody. In some embodiments, the anti-c-met antibody is an IgG2 antibody. In some embodiments, the anti-c-met antibody has a single antigen binding arm specific for c-met.

In some embodiments, the anti-c-met antibody is monovalent. Monovalent antibodies can also be made by methods known in the art for example including, but not limited to, WO 2007/147901 (describing ionic interactions), WO 2007/059782, WO 2007/048037, WO 2008/145137 (nonglycosylated monovalent antibodies), WO 2009/089004 (describing electrostatic steering effects), WO 2010/129304 (describing methods for making heteromultimeric molecules by introducing substitutions in amino acids that are in contact at the interface between polypeptides), WO 2010/063785, WO 2011/133886, and/or WO 2005/063816, which are incorporated herein by reference in their entireties.

In some embodiments, the anti-c-met antibody fragment may comprise a single antigen binding arm and an Fc region. Anti-c-met antibody fragments are described herein and are known in the art, in the one-armed format. Accordingly, in some embodiments, the anti-c-met antibody fragment is a one-armed antibody (i.e., the heavy chain variable domain and the light chain variable domain form a single antigen binding arm) comprising an Fc region, wherein the Fc region comprises a first and a second Fc polypeptide, wherein the first and second Fc polypeptides are present in a complex. In some embodiments, the first and second Fc polypeptides form a Fc region that increases stability of the anti-c-met antibody compared to a Fab molecule comprising said antigen binding arm. In some embodiments, the anti-c-met antibody comprises (a) a first polypeptide comprising the amino acid sequence of SEQ ID NO:19, a CH1 sequence, and a first Fc polypeptide and (b) a second polypeptide comprising the amino acid sequence of SEQ ID NO:20 and CL1 sequence. In some embodiments, the anti-c-met antibody further comprises (c) a third polypeptide comprising a second Fc polypeptide.

In some embodiments, the anti-c-met antibody fragment of the purified anti-c-met antibody compositions and/or for use in the methods of purification comprises an antigen binding site of the bivalent antibody and thus retains the ability to bind antigen. In some embodiments, the anti-c-met antibody fragment comprises the Fc region and retains at least one of the biological functions normally associated with the Fc region when present in an bivalent antibody, such as FcRn binding, antibody half life modulation, ADCC function and complement binding. In some embodiments, the anti-c-met antibody fragment does not have ADCC function and/or complement binding activity. In some embodiments, the anti-c-met antibody fragment is a monovalent antibody that has an in vivo half life substantially similar to a bivalent antibody. For example, such an antibody fragment may comprise on antigen binding arm linked to an Fc sequence capable of conferring in vivo stability to the fragment. In some embodiments, an Fc polypeptide comprises part or all of a wild type hinge sequence (generally at its N terminus). In some embodiments, an Fc polypeptide does not comprise a functional or wild type hinge sequence.

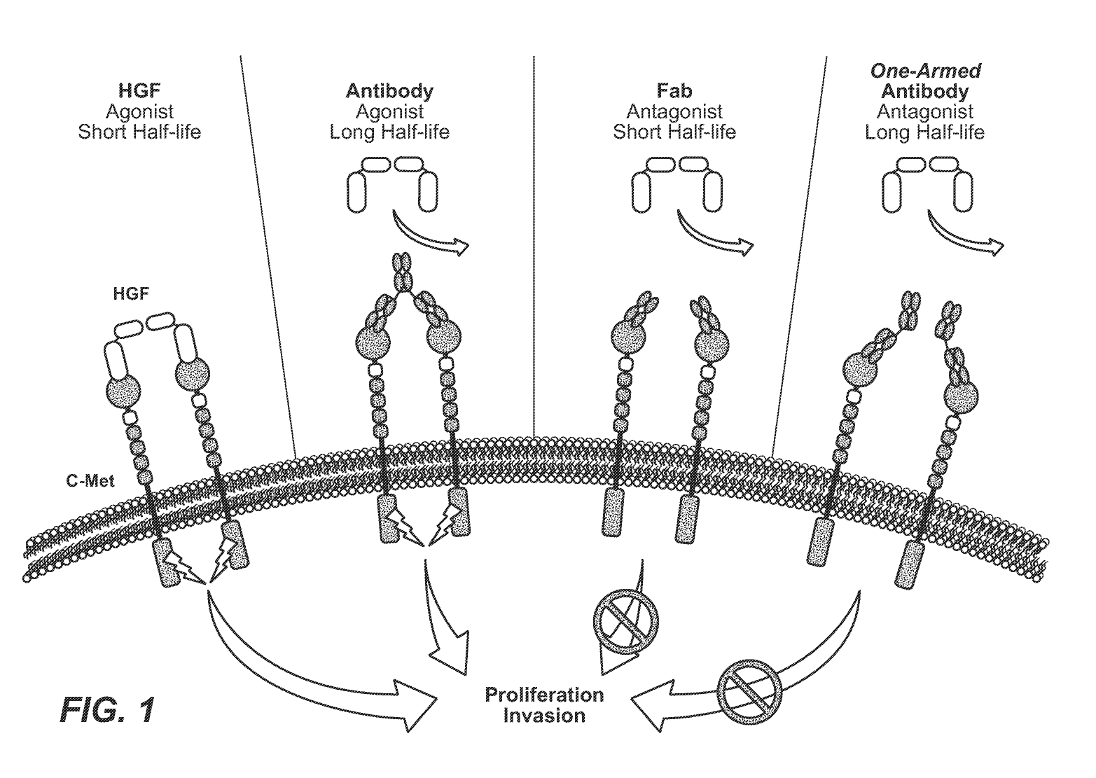

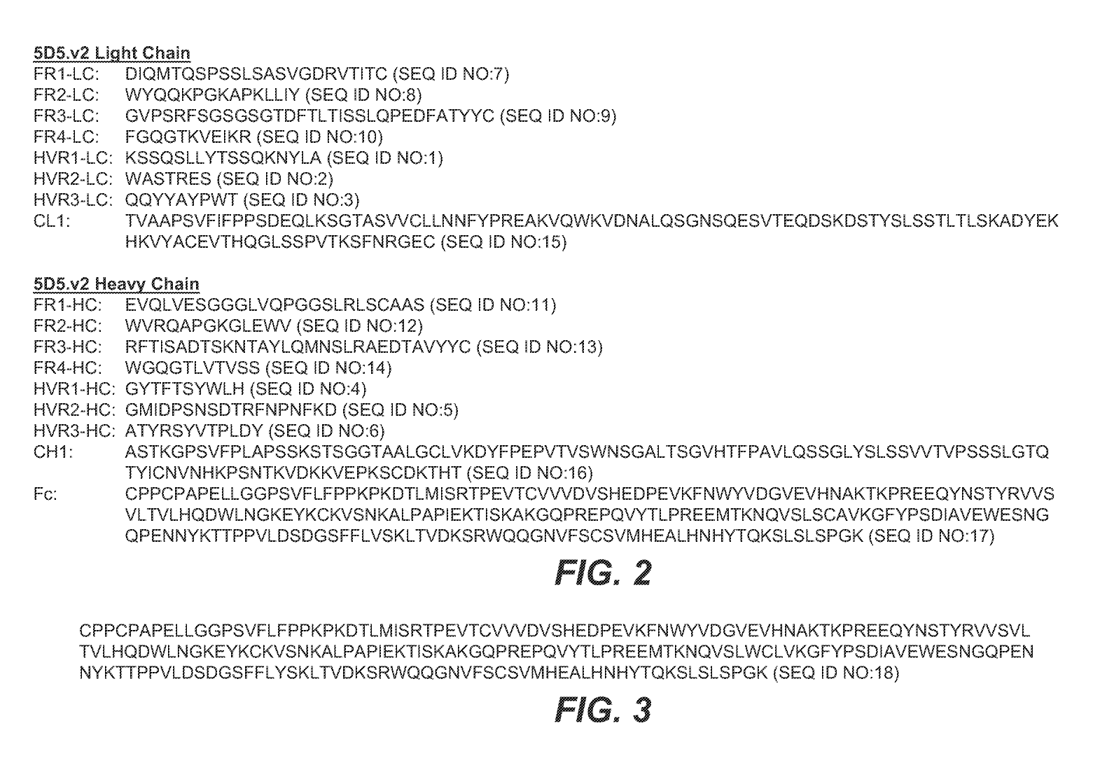

In some embodiments, the anti-c-met antibody fragment is a one-armed antibody as described in WO 2005/063816. In some embodiments, the Fc region of the anti-c-met antibodies comprises a first and a second Fc polypeptide, wherein the first and second polypeptide each comprises one or more mutations with respect to wild type human Fc. In some embodiments, a cavity mutation is T366S, L368A and/or Y407V. In some embodiments, a protuberance mutation is T366W. In some embodiments, the first polypeptide comprises the Fc sequence depicted in FIG. 1 and the second polypeptide comprises the Fc sequence depicted in FIG. 2. In some embodiments, the anti-c-met antibody may comprise at least one characteristic that promotes heterodimerization, while minimizing homodimerization, of the Fc sequences within the antibody fragment.

In some embodiments of any of the purified anti-c-met antibody compositions and/or methods of purification described herein, the anti-c-met antibody is an antagonist anti-c-met antibody. In some embodiments, blocking anti-c-met antibodies or antagonist anti-c-met antibodies completely inhibit the biological activity of the antigen. For treatment of pathological conditions requiring an antagonistic function and where bivalency of an anti-c-met antibody results in an undesirable agonistic effect upon binding to a target antigen (even though it is an antagonistic anti-c-met antibody as a Fab fragment), the monovalent trait of a one-armed antibody (i.e., an antibody comprising a single antigen binding arm) results in and/or ensures an antagonistic function upon binding of the anti-c-met antibody to a target molecule. Furthermore, the one-armed antibody comprising a Fc region is characterized by superior pharmacokinetic attributes (such as an enhanced half life and/or reduced clearance rate in vivo) compared to Fab forms having similar/substantially identical antigen binding characteristics, thus overcoming a major drawback in the use of conventional monovalent Fab antibodies.

Anti-c-met antibodies (which may be provided as one-armed antibodies) of the purified anti-c-met antibodies and/or for use in the methods of purification include those known in the art (see, e.g., Martens, T. et al., Clin. Cancer Res. 12 (20 Pt. 1):6144 (2006); U.S. Pat. No. 6,468,529; WO2006/015371; WO2007/063816, and WO2010/045345, which are incorporated by reference in their entirety). In some embodiments, the anti-c-met antibody of the purified anti-c-met antibodies and/or for use in the methods of purification comprises one or more of the HVR sequences of the monoclonal antibody produced by the hybridoma cell line deposited under American Type Culture Collection (ATCC) Accession Number ATCC HB-11894 (hybridoma 1A3.3.13) or HB-11895 (hybridoma 5D5.11.6). In some embodiments, the anti-c-met antibody is a one-armed antibody comprising one or more of the HVRs of the light chain variable domain and/or one or more of the HVRs of the heavy chain variable domain of ATCC Accession Number ATCC HB-11894 (hybridoma 1A3.3.13) or HB-11895 (hybridoma 5D5.11.6) and an Fc polypeptide.

In some embodiments of any of the purified anti-c-met antibody compositions and/or methods of purification, the anti-c-met antibody comprises a light chain variable domain comprising one or more of HVR1-LC, HVR2-LC and HVR3-LC sequence depicted in FIG. 1 (SEQ ID NOs:1-3). In some embodiments, the anti-c-met antibody comprises a heavy chain variable domain comprising one or more of HVR1-HC, HVR2-HC and HVR3-HC sequence depicted in FIG. 1 (SEQ ID NOs:4-6). In some embodiments, the anti-c-met antibody comprises a light chain variable domain comprising one or more of HVR1-LC, HVR2-LC and HVR3-LC sequence depicted in FIG. 1 (SEQ ID NOs:1-3) and one or more of HVR1-HC, HVR2-HC and HVR3-HC sequence depicted in FIG. 1 (SEQ ID NOs:4-6). In some embodiments, the heavy chain variable domain comprises one or more of HVR1-HC, HVR2-HC and HVR3-HC sequence depicted in FIG. 1 (SEQ ID NOs:4-6) and one or more of FR1-HC, FR2-HC, FR3-HC and FR4-HC sequence depicted in FIG. 1 (SEQ ID NOs:11-14). In some embodiments, the light chain variable domain comprises one or more of HVR1-LC, HVR2-LC and HVR3-LC sequence depicted in FIG. 1 (SEQ ID NOs:1-3) and one or more of FR1-LC, FR2-LC, FR3-LC and FR4-LC sequence depicted in FIG. 1 (SEQ ID NOs:7-10). In some embodiments, the anti-c-met antibody is a one-armed antibody comprising one or more of the HVRs of the light chain variable domain (SEQ ID NOs:1-3) and/or one or more of the HVRs of the heavy chain variable domain (SEQ ID NOs:4-6) and an Fc polypeptide.

In some embodiments of any of the purified anti-c-met antibody compositions and/or methods of purification described herein, the anti-c-met antibody comprises: (a) at least one, two, three, four, or five HVR sequences selected from the group consisting of: (i) HVR-L1 comprising sequence A1-A17, wherein A1-A17 is KSSQSLLYTSSQKNYLA (SEQ ID NO:23) (ii) HVR-L2 comprising sequence B1-B7, wherein B1-B7 is WASTRES (SEQ ID NO:24); (iii) HVR-L3 comprising sequence C1-C9, wherein C1-C9 is QQYYAYPWT (SEQ ID NO:25); (iv) HVR-H1 comprising sequence D1-D10, wherein D1-D10 is GYTFTSYWLH (SEQ ID NO:26); (v) HVR-H2 comprising sequence E1-E18, wherein E1-E18 is GMIDPSNSDTRFNPNFKD (SEQ ID NO:27); and (vi) HVR-H3 comprising sequence F1-F11, wherein F1-F11 is XYGSYVSPLDY (SEQ ID NO:28) and X is not R; and (b) at least one variant HVR, wherein the variant HVR sequence comprises modification of at least one residue of the sequence depicted in SEQ ID NOs:23, 24, 25, 26, 27, or 28. In some embodiments, HVR-L1 of the anti-c-met antibody comprises the sequence of SEQ ID NO:23. In some embodiments, HVR-L2 comprises the sequence of SEQ ID NO:24. In some embodiments, HVR-L3 comprises the sequence of SEQ ID NO:25. In some embodiments, HVR-H1 comprises the sequence of SEQ ID NO:26. In some embodiments, HVR-H2 comprises the sequence of SEQ ID NO:27. In some embodiments, HVR-H3 the sequence of SEQ ID NO:28. In some embodiments, HVR-H3 comprises TYGSYVSPLDY (SEQ ID NO: 29). In some embodiments, HVR-H3 comprises SYGSYVSPLDY (SEQ ID NO:30). In some embodiments, the anti-c-met antibody comprises these sequences (in combination as described herein) is humanized or human. In some embodiments, the anti-c-met antibody is a one-armed antibody comprising one or more of the HVRs of the light chain variable domain (SEQ ID NOs:23-25) and/or one or more of the HVRs of the heavy chain variable domain (SEQ ID NOs:26-30) and an Fc polypeptide.

Provided herein are also anti-c-met antibodies of the purified anti-c-met antibody compositions and/or for use in the methods of purification described herein comprising one, two, three, four, five or six HVRs, wherein each HVR comprises, consists or consists essentially of a sequence selected from the group consisting of SEQ ID NOs:23, 24, 25, 26, 27, 28, and 29, and wherein SEQ ID NO:23 corresponds to an HVR-L1, SEQ ID NO:24 corresponds to an HVR-L2, SEQ ID NO:25 corresponds to an HVR-L3, SEQ ID NO:26 corresponds to an HVR-H1, SEQ ID NO:27 corresponds to an HVR-H2, and SEQ ID NOs:26, 27, or 28 corresponds to an HVR-H3. In some embodiments, the anti-c-met antibody comprises HVR-L1, HVR-L2, HVR-L3, HVR-H1, HVR-H2, and HVR-H3, wherein each, in order, comprises SEQ ID NOs:23, 24, 25, 26, 27 and 29. In some embodiments, the anti-c-met antibody comprises HVR-L1, HVR-L2, HVR-L3, HVR-H1, HVR-H2, and HVR-H3, wherein each, in order, comprises SEQ ID NOs:23, 24, 25, 26, 27 and 30.

Variant HVRs can have modifications of one or more residues within the HVR. In some embodiments, a HVR-L2 variant comprises 1-5 (1, 2, 3, 4 or 5) substitutions in any combination of the following positions: B1 (M or L), B2 (P, T, G or S), B3 (N, G, R or T), B4 (I, N or F), B5 (P, I, L or G), B6 (A, D, T or V) and B7 (R, I, M or G). In some embodiments, a HVR-H1 variant comprises 1-5 (1, 2, 3, 4 or 5) substitutions in any combination of the following positions: D3 (N, P, L, S, A, I), D5 (I, S or Y), D6 (G, D, T, K, R), D7 (F, H, R, S, T or V) and D9 (M or V). In some embodiments, a HVR-H2 variant comprises 1-4 (1, 2, 3 or 4) substitutions in any combination of the following positions: E7 (Y), E9 (I), E10 (I), E14 (T or Q), E15 (D, K, S, T or V), E16 (L), E17 (E, H, N or D) and E18 (Y, E or H). In some embodiments, a HVR-H3 variant comprises 1-5 (1, 2, 3, 4 or 5) substitutions in any combination of the following positions: F1 (T, S), F3 (R, S, H, T, A, K), F4 (G), F6 (R, F, M, T, E, K, A, L, W), F7 (L, I, T, R, K, V), F8 (S, A), F10 (Y, N) and F11 (Q, S, H, F). Letter(s) in parenthesis following each position indicates an illustrative substitution (i.e., replacement) amino acid; as would be evident to one skilled in the art, suitability of other amino acids as substitution amino acids in the context described herein can be routinely assessed using techniques known in the art and/or described herein. In some embodiments, a HVR-L1 comprises the sequence of SEQ ID NO:23. In some embodiments, F1 in a variant HVR-H3 is T. In some embodiments, F1 in a variant HVR-H3 is S. In some embodiments, F3 in a variant HVR-H3 is R. In some embodiments, F3 in a variant HVR-H3 is S. In some embodiments, F7 in a variant HVR-H3 is T. In some embodiments, the anti-c-met antibody comprises a variant HVR-H3 wherein F1 is T or S, F3 is R or S, and F7 is T.

In some embodiments, the anti-c-met antibody of the purified anti-c-met antibody compositions and/or for use in the methods of purification comprises a variant HVR-H3 wherein F1 is T, F3 is R and F7 is T. In some embodiments, the anti-c-met antibody comprises a variant HVR-H3 wherein F1 is S. In some embodiments, the anti-c-met antibody comprises a variant HVR-H3 wherein F1 is T, and F3 is R. In some embodiments, the anti-c-met antibody comprises a variant HVR-H3 wherein F1 is S, F3 is R and F7 is T. In some embodiments, the anti-c-met antibody comprises a variant HVR-H3 wherein F1 is T, F3 is S, F7 is T, and F8 is S. In some embodiments, the anti-c-met antibody comprises a variant HVR-H3 wherein F1 is T, F3 is S, F7 is T, and F8 is A. In some embodiments, said variant HVR-H3 antibody further comprises HVR-L1, HVR-L2, HVR-L3, HVR-H1 and HVR-H2 wherein each comprises, in order, the sequence depicted in SEQ ID NOs:1, 2, 3, 4 and 5. In some embodiments, these antibodies further comprise a human subgroup III heavy chain framework consensus sequence. In some embodiments of these antibodies, the framework consensus sequence comprises substitution at position 71, 73 and/or 78. In some embodiments of these antibodies, position 71 is A, 73 is T and/or 78 is A. In some embodiments of these antibodies, these antibodies further comprise a human κI light chain framework consensus sequence.

In some embodiments, the anti-c-met antibody of the purified anti-c-met antibody compositions and/or for use in the methods of purification comprises a variant HVR-L2 wherein B6 is V. In some embodiments, said variant HVR-L2 anti-c-met antibody further comprises HVR-L1, HVR-L3, HVR-H1, HVR-H2 and HVR-H3, wherein each comprises, in order, the sequence depicted in SEQ ID NOs:23, 25, 26, 27 and 28. In some embodiments, said variant HVR-L2 anti-c-met antibody further comprises HVR-L1, HVR-L3, HVR-H1, HVR-H2 and HVR-H3, wherein each comprises, in order, the sequence depicted in SEQ ID NOs:23, 25, 26, 27 and 29. In some embodiments, said variant HVR-L2 anti-c-met antibody further comprises HVR-L1, HVR-L3, HVR-H1, HVR-H2 and HVR-H3, wherein each comprises, in order, the sequence depicted in SEQ ID NOs:23, 25, 26, 27 and 30. In some embodiments, these anti-c-met antibodies further comprise a human subgroup III heavy chain framework consensus sequence. In some embodiments of these anti-c-met antibodies, the framework consensus sequence comprises substitution at position 71, 73 and/or 78. In some embodiments of these anti-c-met antibodies, position 71 is A, 73 is T and/or 78 is A. In some embodiments of these anti-c-met antibodies, these antibodies further comprise a human κI light chain framework consensus sequence.

In some embodiments, the anti-c-met antibody of the purified anti-c-met antibody compositions and/or for use in the methods of purification comprises a variant HVR-H2 wherein E14 is T, E15 is K and E17 is E. In some embodiments, the anti-c-met antibody comprises a variant HVR-H2 wherein E17 is E. In some embodiments, said variant HVR-H3 anti-c-met antibody further comprises HVR-L1, HVR-L2, HVR-L3, HVR-H1, and HVR-H3 wherein each comprises, in order, the sequence depicted in SEQ ID NOs:23, 24, 25, 26, and 28. In some embodiments, said variant HVR-H2 anti-c-met antibody further comprises HVR-L1, HVR-L2, HVR-L3, HVR-H1, and HVR-H3, wherein each comprises, in order, the sequence depicted in SEQ ID NOs:23, 24, 25, 26, and 29. In some embodiments, said variant HVR-H2 anti-c-met antibody further comprises HVR-L1, HVR-L2, HVR-L3, HVR-H1, and HVR-H3, wherein each comprises, in order, the sequence depicted in SEQ ID NOs:23, 24, 25, 26 and 30. In some embodiments, these anti-c-met antibodies further comprise a human subgroup III heavy chain framework consensus sequence. In some embodiments of these anti-c-met antibodies, the framework consensus sequence comprises substitution at position 71, 73 and/or 78. In some embodiments of these anti-c-met antibodies, position 71 is A, 73 is T and/or 78 is A. In some embodiments of these antibodies, these anti-c-met antibodies further comprise a human κI light chain framework consensus sequence.

In some embodiments, the anti-c-met antibody of the purified anti-c-met antibody compositions and/or for use in the methods of purification comprises (a) a heavy chain variable domain comprising the sequence: EVQLVESGGGLVQPGGSLRLSCAASGYTFTSYWLHWVRQAPGKGLEWVGMIDPSNSDTRFNPN FKDRFTISADTSKNTAYLQMNSLRAEDTAVYYCATYRSYVTPLDYWGQGTLVTVSS (SEQ ID NO:19) and/or (b) a light chain variable domain comprising the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:20). In some embodiments, the anti-c-met antibody is a one-armed antibody comprising (a) a light chain variable domain (SEQ ID NO:20) and/or (b) a heavy chain variable domain (SEQ ID NO:19); and (c) a Fc polypeptide.

In some embodiments, the anti-c-met antibody of the purified anti-c-met antibody compositions and/or for use in the methods of purification comprises (a) HVR-H1, HVR-H2, and HVR-H3 of a heavy chain variable domain comprising the sequence: EVQLVESGGGLVQPGGSLRLSCAASGYTFTSYWLHWVRQAPGKGLEWVGMIDPSNSDTRFNPN FKDRFTISADTSKNTAYLQMNSLRAEDTAVYYCATYRSYVTPLDYWGQGTLVTVSS (SEQ ID NO:19) and/or (b) HVR-L1, HVR-L2, and HVR-L3 of a light chain variable domain comprising the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:20). In some embodiments, the anti-c-met antibody is a one-armed antibody comprising (a) a light chain variable domain (SEQ ID NO:20) and/or (b) a heavy chain variable domain (SEQ ID NO:19); and (c) a Fc polypeptide. In some embodiments, the Fc region is that of a human IgG (e.g., IgG1, 2, 3 or 4). In some embodiments, the first Fc polypeptide comprises the Fc sequence depicted in FIG. 1 (SEQ ID NO:17) and the second Fc polypeptide comprises the Fc sequence depicted in FIG. 2 (SEQ ID NO:18). In some embodiments, the first Fc polypeptide comprises the Fc sequence depicted in FIG. 2 (SEQ ID NO:18) and the second Fc polypeptide comprises the Fc sequence depicted in FIG. 1 (SEQ ID NO:17).

In some embodiments, the anti-c-met antibody of the purified anti-c-met antibody compositions and/or for use in the methods of purification is an anti-c-met antibody fragment, wherein the antibody fragment comprises (a) a first polypeptide comprising a heavy chain variable domain comprising SEQ ID NO:19, CH1 sequence (e.g., SEQ ID NO:16), and a first Fc polypeptide; and (b) a second polypeptide comprising a light chain variable domain comprising SEQ ID NO:20, and CL1 sequence (e.g., SEQ ID NO:15). In some embodiments, the Fc region is that of a human IgG (e.g., IgG1, 2, 3 or 4). In some embodiments, the first Fc polypeptide comprises the Fc sequence depicted in FIG. 1 (SEQ ID NO:17). In some embodiments, the first Fc polypeptide comprises the Fc sequence depicted in FIG. 2 (SEQ ID NO:18).

In some embodiments, the anti-c-met antibody of the purified anti-c-met antibody compositions and/or for use in the methods of purification is an anti-c-met antibody fragment, wherein the antibody fragment comprises (a) a first polypeptide comprising a heavy chain variable domain comprising SEQ ID NO:19, CH1 sequence (e.g., SEQ ID NO:16), and a first Fc polypeptide; (b) a second polypeptide comprising a light chain variable domain comprising SEQ ID NO:20, and CL1 sequence (e.g., SEQ ID NO:15); and (c) a third polypeptide comprising a second Fc polypeptide, wherein the heavy chain variable domain and the light chain variable domain are present as a complex and form a single antigen binding arm and wherein the first and second Fc polypeptides are present in a complex. In some embodiments, the first and second Fc polypeptides form a Fc region that increases stability of said antibody fragment compared to a Fab molecule comprising said antigen binding arm. In some embodiments, the Fc region is that of a human IgG (e.g., IgG1, 2, 3 or 4). In some embodiments, the first Fc polypeptide comprises the Fc sequence depicted in FIG. 1 (SEQ ID NO:17) and the second Fc polypeptide comprises the Fc sequence depicted in FIG. 2 (SEQ ID NO:18). In some embodiments, the first Fc polypeptide comprises the Fc sequence depicted in FIG. 2 (SEQ ID NO:18) and the second Fc polypeptide comprises the Fc sequence depicted in FIG. 1 (SEQ ID NO:17).

In some embodiments, the anti-c-met antibody or anti-c-met antibody fragment thereof, wherein the antibody comprises (a) a first polypeptide comprising a heavy chain variable domain comprising SEQ ID NO:19, CH1 sequence, and a first Fc polypeptide; (b) a second polypeptide comprising a light chain variable domain comprising SEQ ID NO:20, and CL1 sequence; and (c) a third polypeptide comprising a second Fc polypeptide, wherein the heavy chain variable domain and the light chain variable domain are present as a complex and form a single antigen binding arm, wherein the first and second Fc polypeptides are present in a complex and form a Fc region that increases stability of said antibody fragment compared to a Fab molecule comprising said antigen binding arm. In some embodiments, the Fc region is that of a human IgG (e.g., IgG1, 2, 3 or 4). In some embodiments, the first Fc polypeptide comprises the Fc sequence depicted in FIG. 1 (SEQ ID NO:17) and the second Fc polypeptide comprises the Fc sequence depicted in FIG. 2 (SEQ ID NO:18). In some embodiments, the first Fc polypeptide comprises the Fc sequence depicted in FIG. 2 (SEQ ID NO:18) and the second Fc polypeptide comprises the Fc sequence depicted in FIG. 1 (SEQ ID NO:17).

In some embodiments, the anti-c-met antibody comprises (a) a first polypeptide comprising a heavy chain, said polypeptide comprising the sequence: EVQLVESGGGLVQPGGSLRLSCAASGYTFTSYWLHWVRQAPGKGLEWVGMIDPSNSDTRFNPN FKDRFTISADTSKNTAYLQMNSLRAEDTAVYYCATYRSYVTPLDYWGQGTLVTVSSASTKGPS VFPLAPSSKSTSGGTAALGCLVKDYFPEPVTVSWNSGALTSGVHTFPAVLQSSGLYSLSSVVTVP SSSLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGK EYKCKVSNKALPAPIEKTISKAKGQPREPQVYTLPPSREEMTKNQVSLSCAVKGFYPSDIAVEWE SNGQPENNYKTTPPVLDSDGSFFLVSKLTVDKSRWQQGNVFSCSVMHEALHNHYTQKSLSLSPG K (SEQ ID NO:21); (b) a second polypeptide comprising a light chain, the polypeptide comprising the sequence DIQMTQSPSSLSASVGDRVTITCKSSQSLLYTSSQKNYLAWYQQKPGKAPKWYWASTRESGVP SRFSGSGSGTDFTLTISSLQPEDFATYYCQQYYAYPWTFGQGTKVEIKRTVAAPSVFIFPPSDEQL KSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:22); and a third polypeptide comprising a Fc sequence, the polypeptide comprising the sequence DKTHTCPPCPAPELLGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAKGQPREPQ VYTLPPSREEMTKNQVSLWCLVKGFYPSDIAVEWESNGQPENNYKTTPPVLDSDGSFFLYSKLT VDKSRWQQGNVFSCSVMHEALHNHYTQKSLSLSPGK (SEQ ID NO:18). In some embodiments, the heavy chain variable domain and the light chain variable domain are present as a complex and form a single antigen binding arm and wherein the first and second Fc polypeptides are present in a complex. In some embodiments, the first and second Fc polypeptides form a Fc region that increases stability of said antibody fragment compared to a Fab molecule comprising said antigen binding arm.

In some embodiments, the anti-c-met antibody of the purified anti-c-met antibody compositions and/or for use in the methods of purification is a monovalent antibody. In some embodiments, the anti-c-met antibody of the purified anti-c-met antibody compositions and/or for use in the methods of purification is a humanized, human or chimeric antibody.

In some embodiments, polynucleotides encoding any of the anti-c-met antibodies described herein are expressed such that the anti-c-met antibody is produced. In some embodiments, polynucleotides encoding any of the anti-c-met antibody are expressed in vitro or in vivo (for example, in CHO cells or E. coli cells).

In some embodiments, the anti-c-met antibody of the purified anti-c-met antibody compositions and/or for use in the methods of purification described herein is onartuzumab (interchangeably termed MetMAb), a one-armed antibody comprising a Fc region. A sequence of onartuzumab is shown in FIGS. 1 and 2. Onartuzumab (also termed OA5D5v2 and MetMAb) is also described in, e.g., WO2006/015371; WO2010/04345; and Jin et al, Cancer Res (2008) 68:4360. Biosimilar version of onartuzumab are also contemplated and encompassed herein for use in the pharmaceutical formulation.

In some embodiments, the anti-c-met antibody of the purified anti-c-met antibody compositions and/or for use in the methods of purification described herein specifically binds at least a portion of c-met Sema domain or variant thereof. In some embodiments, the anti-c-met antibody is an antagonist. In some embodiments, the anti-c-met antagonist antibody specifically binds at least one of the sequences selected from the group consisting of LDAQT (SEQ ID NO:31) (e.g., residues 269-273 of c-met), LTEKRKKRS (SEQ ID NO:32) (e.g., residues 300-308 of c-met), KPDSAEPM (SEQ ID NO: 33) (e.g., residues 350-357 of c-met) and NVRCLQHF (SEQ ID NO:34) (e.g., residues 381-388 of c-met). In some embodiments, the anti-c-met antagonist antibody specifically binds a conformational epitope formed by part or all of at least one of the sequences selected from the group consisting of LDAQT (SEQ ID NO:31) (e.g., residues 269-273 of c-met), LTEKRKKRS (SEQ ID NO:32) (e.g., residues 300-308 of c-met), KPDSAEPM (SEQ ID NO: 33) (e.g., residues 350-357 of c-met) and NVRCLQHF (SEQ ID NO:34) (e.g., residues 381-388 of c-met). In some embodiments, an antagonist antibody specifically binds an amino acid sequence having at least 50%, 60%, 70%, 80%, 90%, 95%, 98% sequence identity or similarity with the sequence LDAQT (SEQ ID NO:31), LTEKRKKRS (SEQ ID NO:32), KPDSAEPM (SEQ ID NO:33) and/or NVRCLQHF (SEQ ID NO:34). In some embodiments, the anti-c-met antibody is an antagonist anti-c-met antibody. In some embodiments, the anti-c-met antibody is a one-armed antibody. In order to screen for antibodies which bind to an epitope on an antigen bound by an antibody of interest, a routine cross-blocking assay such as that described in Antibodies, A Laboratory Manual, Cold Spring Harbor Laboratory, Ed Harlow and David Lane (1988), can be performed.

Other anti-c-met antibodies suitable for use in the methods of the invention are described herein and known in the art. For example, anti-c-met antibodies disclosed in WO05/016382 (including but not limited to antibodies 13.3.2, 9.1.2, 8.70.2, 8.90.3); an anti-c-met antibodies produced by the hybridoma cell line deposited with ICLC number PD 03001 at the CBA in Genoa, or that recognizes an epitope on the extracellular domain of the β chain of the HGF receptor, and said epitope is the same as that recognized by the monoclonal antibody); anti-c-met antibodies disclosed in WO2007/126799 (including but not limited to 04536, 05087, 05088, 05091, 05092, 04687, 05097, 05098, 05100, 05101, 04541, 05093, 05094, 04537, 05102, 05105, 04696, 04682); anti c-met antibodies disclosed in WO2009/007427 (including but not limited to an antibody deposited at CNCM, Institut Pasteur, Paris, France, on Mar. 14, 2007 under the number I-3731, on Mar. 14, 2007 under the number I-3732, on Jul. 6, 2007 under the number I-3786, on Mar. 14, 2007 under the number I-3724); an anti-c-met antibody disclosed in 20110129481; an anti-c-met antibody disclosed in US20110104176; an anti-c-met antibody disclosed in WO2009/134776; an anti-c-met antibody disclosed in WO2010/059654; an anti-c-met antibody disclosed in WO2011/020925 (including but not limited to an antibody secreted from a hybridoma deposited at the CNCM, Institut Pasteur, Paris, France, on Mar. 12, 2008 under the number I-3949 and the hybridoma deposited on Jan. 14, 2010 under the number I-4273); an anti-c-met antibody disclosed in WO 2011/110642; an anti-c-met antibody disclosed in WO 2011/090754; an anti-c-met antibody disclosed in WO2007/090807; an anti-c-met antibody disclosed in WO2012059561A1.

In some embodiments, the anti-c-met antibody is a monovalent antibody comprising heterodimers of a first protein chain comprising the variable domain of the heavy chain of an antibody of interest and the CH2 and CH3 domains of an IgG and a second protein chain comprising the variable domain of the light chain of the antibody of interest and the CH2 and CH3 domains of said IgG. In some embodiments, the anti-c-met antibody is a monovalent antibody comprising a light chain comprising a variable light chain region and a constant light chain region, wherein the constant light chain region is modified so that it does not contain amino acid capable of forming disulfide bonds. In some embodiments, the anti-c-met antibody is a monovalent antibody comprising a variable heavy chain region and a constant heavy chain region, wherein the constant heavy chain region is modified so that it does not contain amino acid capable of forming disulfide bonds. In some embodiments, the anti-c-met antibody is a monovalent antibody comprising knobs:holes-type mutations. In some embodiments, the anti-c-met antibody is a monovalent antibody comprising one or more CH3 mutations selected from the group consisting of R238Q, R238Q, D239E, K292R, Q302E, P328L, R285Q, S314N, N322K, M327V, K339R, Q349E, 1352V, R365H, F366Y, and P375L. In some embodiments, the anti-c-met antibody is a monovalent antibody comprising a light chain-Fc fusion. In some embodiments, the anti-c-met antibody is a monovalent antibody comprising a hinge deletion.

In some embodiments of any of the purified anti-c-met antibody compositions and/or methods of purification described herein, the anti-c-met antibody may interfere with HGF/c-met activation, including but not limited to interfering with HGF binding to the extracellular portion of c-met and receptor multimerization. In some embodiments, the anti-c-met antibody are useful in treating or diagnosing pathological conditions associated with abnormal or unwanted signaling of the HGF/c-met pathway. In some embodiments, the anti-c-met antibody may modulate the HGF/c-met pathway, including modulation of c-met ligand binding, c-met dimerization, activation, and other biological/physiological activities associated with HGF/c-met signaling. In some embodiments, the anti-c-met antibody may disrupt HGF/c-met signaling pathway. In some embodiments of any of the anti-c-met antibodies described herein, binding of the anti-c-met antibody to c-met inhibits c-met activation by HGF. In some embodiments of any of the anti-c-met antibodies, binding of the anti-c-met antibody to c-met in a cell inhibits proliferation, survival, scattering, morphogenesis and/or motility of the cell.

In some instances, it may be advantageous to have an anti-c-met antibody that does not interfere with binding of a ligand (such as HGF) to c-met. Accordingly, in some embodiments, the anti-c-met antibody does not bind an HGF binding site on c-met. In some embodiment, the anti-c-met antibody does not substantially inhibit HGF binding to c-met. In some embodiments, the anti-c-met antibody does not substantially compete with HGF for binding to c-met. In one example, the anti-c-met antibody can be used in conjunction with one or more other antagonists, wherein the antagonists are targeted at different processes and/or functions within the HGF/c-met axis. Thus, in some embodiments, the anti-c-met antibody binds to an epitope on c-met distinct from an epitope bound by another c-met antagonist (such as the Fab fragment of the monoclonal antibody produced by the hybridoma cell line deposited under American Type Culture Collection Accession Number ATCC HB-11894 (hybridoma 1A3.3.13)). In another embodiment, the anti-c-met antibody is distinct from (i.e., it is not) a Fab fragment of the monoclonal antibody produced by the hybridoma cell line deposited under American Type Culture Collection Accession Number ATCC HB-11894 (hybridoma 1A3.3.13).

In some embodiments, the anti-c-met antibody binds to c-met of a first animal species, and does not specifically bind to c-met of a second animal species. In some embodiments, the first animal species is human and/or primate (e.g., cynomolgus monkey), and the second animal species is murine (e.g., mouse) and/or canine. In some embodiments, the first animal species is human. In some embodiments, the first animal species is primate, for example cynomolgus monkey. In some embodiments, the second animal species is murine, for example mouse. In some embodiments, the second animal species is canine.

In some embodiments, the anti-c-met antibody elicits little to no immunogenic response in said subject. In some embodiments, the anti-c-met antibody elicits an immunogenic response at or less than a clinically-acceptable level.

In some embodiments of any of the purified anti-c-met antibody compositions and/or methods of purification, an altered antibody that possesses some but not all effector functions. In some embodiments, the anti-c-met antibody does not possess complement depletion and/or ADCC activity. In some embodiments, the Fc activities of the produced immunoglobulin are measured to ensure that only the desired properties are maintained (e.g., half-life but not complement depletion and/or ADCC activity). In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol 9:457-92 (1991). An example of an in vitro assay to assess ADCC activity of a molecule of interest is described in U.S. Pat. No. 5,500,362 or U.S. Pat. No. 5,821,337. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. PNAS (USA) 95:652-656 (1998). C1q binding assays may also be carried out to confirm that the antibody is unable to bind C1q and hence lacks CDC activity. To assess complement activation, a CDC assay, e.g. as described in Gazzano-Santoro et al., J. Immunol. Methods 202:163 (1996), may be performed. FcRn binding and in vivo clearance/half life determinations can also be performed using methods known in the art. In some embodiments, the anti-c-met antibody is glycosylated. In some embodiments, the anti-c-met antibody is substantially aglycosylated.

The anti-c-met antibodies of the purified anti-c-met antibody compositions and/or for use in the methods of purification can be characterized for their physical/chemical properties and biological functions by various assays known in the art. The purified anti-c-met antibodies can be further characterized by a series of assays including, but not limited to, N-terminal sequencing, amino acid analysis, non-denaturing size exclusion high pressure liquid chromatography (HPLC), mass spectrometry, ion exchange chromatography and papain digestion.

In some embodiments of any of the purified anti-c-met antibody compositions and/or methods of purification described herein, the anti-c-met antibody may be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue, or silver stain.

Further, in some embodiments of any of the purified anti-c-met antibody compositions and/or methods of purification described herein, the anti-c-met antibody may incorporate any of the features, singly or in combination, as described in Sections 1-8 below:

1. Antibody Affinity

In some embodiments, the anti-c-met antibody has a dissociation constant (Kd) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³ M, e.g., from 10⁻⁹M to 10⁻¹³ M).

Binding affinity of a ligand to its receptor can be determined using any of a variety of assays, and expressed in terms of a variety of quantitative values. Antigen binding assays are known in the art and can be used herein include without limitation any direct or competitive binding assays using techniques such as western blots, radioimmunoassays, enzyme-linked immunoabsorbent assay (ELISA), “sandwich” immunoassays, surface plasmon resonance based assay (such as the BIAcore assay as described in PCT Application Publication No. WO2005/012359), immunoprecipitation assays, fluorescent immunoassays, and protein A immunoassays.

Accordingly, in some embodiments, the binding affinity is expressed as Kd values and reflects intrinsic binding affinity (e.g., with minimized avidity effects). The anti-c-met antibody selected will normally have a sufficiently strong binding affinity for c-met, for example, the antibody may bind human c-met with a Kd value of between 100 nM⁻¹ pM.

2. Antibody Fragments

In some embodiments, the anti-c-met antibody is an antibody fragment. Antibody fragments include, but are not limited to, Fab, Fab′, Fab′-SH, F(ab′)₂, Fv, one-armed antibodies, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Other monovalent antibody forms are described in, e.g., WO2007048037, WO2008145137, WO2008145138, and WO2007059782. One-armed antibodies are described, e.g., in WO2005/063816. Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In some embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In some embodiments, the anti-c-met antibody is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-HVR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In some embodiments, the anti-c-met antibody is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

The anti-c-met antibody may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004).

In some phage display methods, repertoires of V_{H} and V_{L} genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In some embodiments, the anti-c-met antibody is a multispecific antibody, e.g. a bispecific antibody. Multispecific antibodies are monoclonal antibodies that have binding specificities for at least two different sites. In some embodiments, one of the binding specificities is for an antigen and the other is for any other antigen. In some embodiments, bispecific antibodies may bind to two different epitopes of an antigen. Bispecific antibodies may also be used to localize cytotoxic agents to cells which express an antigen. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to c-met as well as another, different antigen (see, US 2008/0069820, for example).

7. Antibody Variants

In some embodiments, amino acid sequence variants of the anti-c-met antibody are contemplated. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a. Substitution, Insertion, and Deletion Variants

In some embodiments, anti-c-met antibody variants having one or more amino acid substitutions are provided. Sites of interest for substitutional mutagenesis include the HVRs and FRs. Conservative substitutions are shown in Table 1 under the heading of “conservative substitutions.” More substantial changes are provided in Table 1 under the heading of “exemplary substitutions,” and as further described below in reference to amino acid side chain classes Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or SDRs (a-CDRs), with the resulting variant V_{H} or V_{L} being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized HVR residues involved in antigen binding may be specifically identified, e.g., using alanine scanning mutagenesis or modeling. CDR-H3 and CDR-L3 in particular are often targeted.

In some embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may be outside of HVR “hotspots” or SDRs. In some embodiments of the variant V_{H} and V_{L} sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b. Glycosylation Variants

In some embodiments, the anti-c-met antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody may be made in order to create antibody variants with certain improved properties.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c. Fc Region Variants

In some embodiments, one or more amino acid modifications may be introduced into the Fc region of the anti-c-met antibody, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In some embodiments, contemplated are antibody variants that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In some embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d. Cysteine Engineered Antibody Variants

In some embodiments, it may be desirable to create cysteine engineered antibodies, e.g., “thioMAbs,” in which one or more residues of the anti-c-met antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In some embodiments, any one or more of the following residues may be substituted with cysteine: V205 (Kabat numbering) of the light chain; A118 (EU numbering) of the heavy chain; and 5400 (EU numbering) of the heavy chain Fc region. Cysteine engineered antibodies may be generated as described, e.g., in U.S. Pat. No. 7,521,541.

e. Antibody Derivatives

In some embodiments, the anti-c-met antibody may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of the anti-c-met antibody and nonproteinaceous moiety that may be selectively heated by exposure to radiation are provided. In some embodiments, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

8. Immunoconjugates

Immunoconjugates comprising the anti-c-met antibody conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes are contemplated for use in the purified anti-c-met antibody compositions and/or methods of purification described herein.

In some embodiments, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some embodiments, an immunoconjugate comprises the anti-c-met antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In some embodiments, an immunoconjugate comprises the anti-c-met antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of the anti-c-met antibody and cytotoxic agent may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### V. PHARMACEUTICAL FORMULATIONS

Provided herein are also pharmaceutical formulations comprising the purified anti-c-met antibody compositions and/or antibodies purified by the methods described herein. In some embodiments, the pharmaceutical formulation is a stable liquid pharmaceutical formulation. In some embodiments, the anti-c-met antibody is an antagonist anti-c-met antibody. In some embodiments, the pharmaceutical formulation is a liquid pharmaceutical formulation. In some embodiments, the pharmaceutical formulation is suitable for administration to an individual (e.g., human)

In some embodiments of any of the pharmaceutical formulations, the HCP in the pharmaceutical formulation comprising a composition comprising the anti-c-met antibody is less than or equal to about 50 ng/mg. In some embodiments of any of the pharmaceutical formulations, the average HCP in a lot (e.g., batch) of the pharmaceutical formulation comprising a composition comprising the anti-c-met antibody is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the DNA levels in the composition comprising an anti-c-met antibody are less than or equal to about 0.3 pg/mg. In some embodiments of any of the pharmaceutical formulations, the average DNA levels in a lot (e.g., batch) of the composition comprising an anti-c-met antibody are less than or equal to about 0.3 pg/mg. In some embodiments, the DNA levels and/or average DNA levels are less than or equal to about any of 0.3 pg/mg, 0.25 pg/mg, 0.2 pg/mg, 0.15 pg/mg, or 0.1 pg/mg. In some embodiments, the DNA levels and/or average DNA levels are between about any of 0.001 pg/mg and 0.3 pg/mg, 0.001 pg/mg and 0.2 pg/mg, 0.001 pg/mg and 0.1 pg/mg, 0.01 pg/mg and 0.3 pg/mg, 0.01 pg/mg and 0.2 pg/mg, or 0.01 pg/mg and 0.1 pg/mg. In some embodiments, the DNA levels and/or average DNA levels are about any of 0.3, 0.25, 0.2, 0.15, or 0.1 pg/mg. In some embodiments, DNA levels are determined by PCR. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the leached protein A (LpA) in the composition comprising an anti-c-met antibody is less than or equal to about 2 ng/mg. In some embodiments of any of the pharmaceutical formulations, the average LpA in a lot (e.g., batch) of the composition comprising an anti-c-met antibody is less than or equal to about 2 ng/mg. In some embodiments, the LpA and/or average LpA is between about any of 0.001 ng/mg and 2 ng/mg, 0.01 ng/mg and 2 ng/mg, 0.1 ng/mg and 2 ng/mg, or 1 ng/mg and 2 ng/mg. In some embodiments, the LpA and/or average LpA is about any of 1, 1.25, 1.5, 1.75, or 2 ng/mg. In some embodiments, percentage of LpA is determined by leached protein A ligand assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the Limulus Amebocyte Lysate (LAL) in the composition comprising an anti-c-met antibody is less than or equal to about 0.01 EU/mg. In some embodiments of any of the pharmaceutical formulations, the average LAL in a lot (e.g., batch) of the composition comprising an anti-c-met antibody is less than or equal to about 0.01 EU/mg. In some embodiments, the LAL and/or average LAL is less than or equal to about any of 0.007 EU/mg, 0.006 EU/mg, 0.005 EU/mg, 0.002 EU/mg, or 0.001 EU/mg. In some embodiments, the LAL and/or average LAL is between about any of 0.0001 EU/mg and 0.01 EU/mg, 0.0001 EU/mg and 0.007 EU/mg, 0.0001 EU/mg and 0.006 EU/mg, or 0.0001 EU/mg and 0.005 EU/mg. In some embodiments, the LAL and/or average LAL is about any of 0.01, 0.007, 0.006, 0.005, 0.004, 0.003, or 0.002 EU/mg. In some embodiments, percentage of LAL is determined by LAL assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the percentage of aggregates in the composition comprising an anti-c-met antibody is less than or equal to about 0.3%. In some embodiments of any of the pharmaceutical formulations, the average percentage of aggregates in a lot (e.g., batch) of the composition comprising an anti-c-met antibody is less than or equal to about 0.3%. In addition, provided herein are pharmaceutical formulations comprising a composition comprising an anti-c-met antibody, wherein percentage of aggregates present in the composition is less than or equal to about 0.3%. Further provided herein are pharmaceutical formulations comprising a lot (e.g., batch) of a composition comprising an anti-c-met antibody, wherein the average percentage of aggregates present in the composition is less than or equal to about 0.3%. In some embodiments, the percentage of aggregates and/or average percentage of aggregates is less than or equal to about any of 0.2% or 0.1%. In some embodiments, the percentage of aggregates and/or average percentage of aggregates is between about any of 0.001% and 0.3%, 0.01% and 0.3%, 0.001% and 0.2%, or 0.01% and 0.2%. In some embodiments, the percentage of aggregates and/or average percentage of aggregates is about any of 0.3%, 0.25%, 0.2%, 0.15%, or 0.1%. In some embodiments, percentage of aggregates is determined by size exclusion chromatography (SEC) assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the percentage of monomer in the composition comprising an anti-c-met antibody is greater than or equal to about 99.5%. In some embodiments of any of pharmaceutical formulations, the average percentage monomer in a lot (e.g., batch) of the composition comprising an anti-c-met antibody is greater than or equal to about 99.5%. In addition, provided herein are pharmaceutical formulations comprising a composition comprising an anti-c-met antibody, wherein the percentage of monomer present in the composition is greater than or equal to about 99.5%. Further provided herein are pharmaceutical formulations comprising a lot (e.g., batch) of a composition comprising an anti-c-met antibody, wherein the average percentage of monomer present in the composition is greater than or equal to about 0.3%. In some embodiments, the percentage of monomer and/or average percentage of monomer is greater than or equal to about any of 99.6%, 99.7%, 99.8%, or 99.9%. In some embodiments, the percentage of monomer and/or average percentage of monomer is between about any of 99.5% and 99.999%, 99.5% and 99.99%, 99.6% and 99.999%, 99.6% and 99.99%, 99.7% and 99.999%, 99.7% and 99.99%, 99.8% and 99.999%, 99.8% and 99.99%, or 99.9% and 99.999%, 99.9% and 99.99%. In some embodiments, the percentage of monomer and/or average percentage of monomer is about any of 99.5%, 99.6%, 99.7%, 99.8%, or 99.9%. In some embodiments, percentage of monomer is determined by SEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the percentage of fragments in the composition comprising an anti-c-met antibody is less than or equal to about 0.3%. In some embodiments of any of the pharmaceutical formulations, the average percentage of fragments in a lot (e.g., batch) of the composition comprising an anti-c-met antibody is less than or equal to about 0.3%. In addition, provided herein are pharmaceutical formulations comprising a composition comprising an anti-c-met antibody, wherein percentage of fragments present in the composition is less than or equal to about 0.3%. Further provided herein are pharmaceutical formulations comprising a lot (e.g., batch) of a composition comprising an anti-c-met antibody, wherein the average percentage of fragments present in the composition is less than or equal to about 0.3%. In some embodiments, the percentage of fragments and/or average percentage of fragments is less than or equal to about any of 0.2% or 0.1%. In some embodiments, the percentage of fragments and/or average percentage of fragments is between about any of 0.001% and 0.3%, 0.01% and 0.3%, 0.001% and 0.2%, or 0.01% and 0.2%. In some embodiments, the percentage of fragments and/or average percentage of fragments is about any of 0.3%, 0.25%, 0.2%, 0.15%, 0.1%, or 0%. In some embodiments, fragments are not detectable. In some embodiments, percentage of fragments is determined by SEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the percentage of acidic variants in the composition comprising an anti-c-met antibody is less than or equal to about 20%. In some embodiments of any of the pharmaceutical formulations, the average percentage of acidic variants in a lot (e.g., batch) of the composition comprising an anti-c-met antibody is less than or equal to about 20%. In addition, provided herein are pharmaceutical formulations comprising a composition comprising an anti-c-met antibody, wherein percentage of acidic variants present in the composition is less than or equal to about 20%. Further provided herein are a pharmaceutical formulation comprising a lot (e.g., batch) of a composition comprising an anti-c-met antibody, wherein the average acidic variants present in the composition is less than or equal to about 20%. In some embodiments, the percentage of acidic variants and/or average percentage of acidic variants is less than or equal to about any of 20%, 18.5%, 17.5%, 15%, 12.5%. In some embodiments, the percentage of acidic variants and/or average percentage of acidic variants is between about any of 1% and 20%, 5% and 20%, or 10% and 20%. In some embodiments, the percentage of acidic variants and/or average percentage of acidic variants is about any of 20%, 18.5%, 17.5%, 15%, or 12.5%. In some embodiments, percentage of acidic variants is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the pharmaceutical formulations, the percentage of main peak in the composition comprising an anti-c-met antibody is greater than or equal to about 75%. In some embodiments of any of the pharmaceutical formulations, the average percentage of main peak in a lot (e.g., batch) of the composition comprising an anti-c-met antibody is greater than or equal to about 75%. In addition, provided herein are pharmaceutical formulations comprising a composition comprising an anti-c-met antibody, wherein percentage of main peak present in the composition is greater than or equal to about 75%. Further provided herein are pharmaceutical formulations comprising a lot (e.g., batch) of a composition comprising an anti-c-met antibody, wherein the average percentage of main peak present in the composition is greater than or equal to about 75%. In some embodiments, the percentage of main peak and/or average percentage of main peak greater than or equal to about any of 77.5%, 80%, 82.5%, or 85%. In some embodiments, the percentage of main peak and/or average percentage of main peak is between about any of 75% and 95%, 77.5% and 95%, 80% and 95%, 82.5% and 95%, or 85% and 95%. In some embodiments, the percentage of main peak and/or average percentage of main peak is about any of 75%, 77.5%, 80%, 82.5%, or 85%. In some embodiments, percentage of main peak is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

In some embodiments of any of the formulations, the percentage of basic variants in the composition comprising an anti-c-met antibody is less than or equal to about 2.0%. In some embodiments of any of the pharmaceutical formulations, the average percentage of basic variants in a lot (e.g., batch) of the composition comprising an anti-c-met antibody is less than or equal to about 2.0%. In addition, provided herein are pharmaceutical formulations comprising a composition comprising an anti-c-met antibody, wherein percentage of basic variants present in the composition is less than or equal to about 2.0%. Further provided herein are pharmaceutical formulations comprising a lot (e.g., batch) of a composition comprising an anti-c-met antibody, wherein the average percentage of basic variants present in the composition is less than or equal to about 2.0%. In some embodiments, the percentage of basic variants and/or average percentage of basic variants is less than or equal to about any of 1.5%, 1.25%, 1.1%, or 1%. In some embodiments, the percentage of basic variants and/or average percentage of basic variants is between about any of 0.001% and 2%, 0.01% and 2%, 0.001% and 1.5%, or 0.01% and 1.5%, 0.001% and 1.0%, or 0.01% and 1.0%. In some embodiments, the percentage of basic variants and/or average percentage of basic variants is about any of 2%, 1.5%, 1.25%, 1.1%, or 1%. In some embodiments, percentage of basic variants is determined by HPIEC assay. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

Pharmaceutical formulations are prepared by mixing such antibody having the desired degree of purity with one or more optional pharmaceutically acceptable carriers such as those described in Remington's Pharmaceutical Sciences 18th edition, Gennaro, A. Ed. (1990) in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The pharmaceutical formulation to be used for in vivo administration should be sterile. This can be achieved according to the procedures known to the skilled person for generating sterile pharmaceutical formulations suitable for administration to human subjects, including filtration through sterile filtration membranes, prior to, or following, preparation of the formulation.

The pharmaceutical formulation herein may also contain more than one active compound as necessary for the particular indication being treated, preferably those with complementary activities that do not adversely affect each other. Such molecules are suitably present in combination in amounts that are effective for the purpose intended.

In some embodiments, the pharmaceutical formulation comprises a composition comprising a purified anti-c-met antibody and/or an antibody purified by a method described herein, a polysorbate, a saccharide, and a buffer. Examples of polysorbate include, but are not limited to, polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and/or polysorbate 80 (polyoxyethylene (20) sorbitan monooleate). Saccharides include, but are not limited to, glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc. Examples of histidine buffers include, but are not limited to, histidine chloride, histidine succinate, histidine acetate, histidine phosphate, histidine sulfate. In some embodiments, the pharmaceutical formulation comprises (a) a composition comprising a purified anti-c-met antibody (e.g., onartuzumab) and/or anti-c-met antibody purified by a process described herein, wherein the anti-c-met antibody is present at a concentration between about 50 mg/mL and about 75 mg/mL; (b) a histidine acetate buffer at pH 5.0-5.4, wherein the histidine acetate buffer is at a concentration between about 1 mM and about 20 mM; (c) sucrose, wherein the sucrose is at a concentration between about 100 mM to about 150 mM; and (d) polysorbate 20, wherein the polysorbate 20 concentration is greater than 0.02% w/v. In some embodiments, the pharmaceutical formulation comprises (a) a composition comprising a purified anti-c-met antibody (e.g., onartuzumab) and/or anti-c-met antibody purified by a process described herein, wherein the anti-c-met antibody is present at a concentration of about 60 mg/mL; (b) a histidine acetate buffer at pH 5.4, wherein the histidine acetate buffer is at a concentration of about 10 mM; (c) sucrose, wherein the sucrose is at a concentration of about 120 mM; and (d) polysorbate 20, wherein the polysorbate 20 concentration is about 0.04% w/v. In some embodiments, the pharmaceutical formulation is diluted prior to administration (e.g., diluted to 1 mg/mL in saline).

Further, provided herein are vials and methods of filing a vial comprising the pharmaceutical formulation. In some embodiments, the pharmaceutical formulation is provided inside a vial with a stopper pierceable by a syringe, preferably in aqueous form. The vial is desirably stored at about 2-8° C. as well as up to 30° C. for 24 hours until it is administered to a subject in need thereof. The vial may for example be a 15 cc vial (for example for a 600 mg dose) or 20 cc vial (for example for a 900 mg dose).

### VI. USES AND METHODS OF TREATMENT

The purified anti-c-met antibody compositions, pharmaceutical formulations comprising purified anti-c-met antibody compositions, and/or anti-c-met antibodies purified by the methods provided herein comprising are useful for modulating disease states associated with dysregulation of the HGF/c-met signaling axis. The HGF/c-met signaling pathway is involved in multiple biological and physiological functions, including, e.g., cell proliferation and angiogenesis.

Provided herein are methods of inhibiting c-met activated cell proliferation, said method comprising contacting a cell or tissue with a purified anti-c-met antibody composition, a pharmaceutical formulation comprising a purified anti-c-met antibody composition, and/or anti-c-met antibody purified by the methods described herein comprising an effective amount of an anti-c-met antibody, whereby cell proliferation associated with c-met activation is inhibited. In some embodiments, the cell proliferative disorder is associated with increased expression or activity of c-met or hepatocyte growth, or both. In some embodiments, the cancer is c-met positive (expresses high levels of c-met, for example, by immunohistochemistry). In some embodiments, the cell proliferation is cancer. In some embodiments, the cancer is non-small cell lung cancer (NSCLC), glioblastoma, pancreatic cancer, sarcoma, renal cell carcinoma, hepatocellular carcinoma, gastric cancer, colorectal cancer, or breast cancer. In some embodiments, the cancer is stage IIIb and/or stage IV. In some embodiments, the cancer is locally advanced or metastatic cancer. In some embodiments, the therapy is second line or third line therapy (e.g., second line or third line NSCLC therapy). In some embodiments, the cancer is EGFR mutant. In some embodiments, the cancer is EGFR wild-type. In some embodiments, the cancer is c-met positive (expresses high levels of c-met, for example, by immunohistochemistry (IHC)).

Provided herein are methods of treating a pathological condition associated with dysregulation of c-met activation in a subject, said method comprising administering to the subject a purified anti-c-met antibody composition, a pharmaceutical formulation comprising a purified anti-c-met antibody composition, and/or anti-c-met antibody purified by the methods described herein comprising an effective amount of the c-met antibody, whereby said condition is treated. In some embodiments, the pathological condition is cancer. In some embodiments, the cancer is non-small cell lung cancer (NSCLC), glioblastoma, pancreatic cancer, sarcoma, renal cell carcinoma, hepatocellular carcinoma, gastric cancer, colorectal cancer, or breast cancer. In some embodiments, the cancer is stage IIIb and/or stage IV cancer. In some embodiments, the cancer is locally advanced or metastatic cancer. In some embodiments, the therapy is second line or third line therapy (e.g., second line or third line NSCLC therapy). Dysregulation of c-met activation (and thus signaling) can result from a number of cellular changes, including, for example, overexpression of HGF (c-met's cognate ligand) and/or c-met itself. In some embodiments, the cancer is EGFR mutant. In some embodiments, the cancer is EGFR wild-type. In some embodiments, the cancer is c-met positive (expresses high levels of c-met, for example, by IHC).

Also provided herein are methods of inhibiting the growth of a cell that expresses c-met or hepatocyte growth factor, or both, said method comprising contacting said cell with a purified anti-c-met antibody composition, a pharmaceutical formulation comprising a purified anti-c-met antibody composition, and/or antibody purified by the methods described herein comprising an anti-c-met antibody thereby causing an inhibition of growth of said cell. In some embodiments, the growth of said cell is at least in part dependent upon a growth potentiating effect of c-met or hepatocyte growth factor, or both. In some embodiments, the cell is contacted by HGF expressed by a different cell (e.g., through a paracrine effect).

Provided herein are also methods for treating or preventing cancer comprising administering a purified anti-c-met antibody (e.g., onartuzumab) composition, a pharmaceutical formulation comprising a purified anti-c-met antibody composition, and/or anti-c-met antibody purified by the methods described herein. In some embodiments, the cancer is non-small cell lung cancer (NSCLC), glioblastoma, pancreatic cancer, sarcoma, renal cell carcinoma, hepatocellular carcinoma, gastric cancer, colorectal cancer, or breast cancer. In some embodiments, the cancer is stage IIIb and/or stage IV cancer. In some embodiments, the cancer is locally advanced or metastatic cancer. In some embodiments, the therapy is second line or third line therapy (e.g., second line or third line NSCLC therapy). In some embodiments, the cancer is EGFR mutant. In some embodiments, the cancer is EGFR wild-type. In some embodiments, the cancer is c-met positive (expresses high levels of c-met, for example, by IHC). In some embodiments, the dose of anti-c-met antibody is about 15 mg/kg. In some embodiments, the dose of anti-c-met antibody is about 15 mg/kg administered day one of a 21 day cycle. In some embodiments, the dose of anti-c-met antibody is about 10 mg/kg. In some embodiments, the dose of anti-c-met antibody is about 10 mg/kg administered on day 1 and 15 of a 28 day cycle.

In some embodiments of any of the methods, the HCP in the composition comprising the anti-c-met antibody and/or the pharmaceutical formulation comprising the purified anti-c-met antibody composition is less than or equal to about 50 ng/mg. In some embodiments of any of the methods, the average HCP in a lot (e.g., batch) of the composition comprising the anti-c-met antibody and/or a lot (e.g., batch) of the pharmaceutical formulation comprising the purified anti-c-met antibody composition is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.2, about 8.3, and/or about 8.4. In some embodiments, the anti-c-met antibody is onartuzumab.

Methods described herein can be used to affect any suitable pathological state, for example, cells and/or tissues associated with dysregulation of the HGF/c-met signaling pathway. In some embodiments of any of the methods described herein, a cell that is targeted in a method described herein is a cancer cell. For example, a cancer cell can be one selected from the group consisting of a breast cancer cell, a colorectal cancer cell, a lung cancer cell, a papillary carcinoma cell (e.g., of the thyroid gland), a colon cancer cell, a pancreatic cancer cell, an ovarian cancer cell, a cervical cancer cell, a central nervous system cancer cell, an osteogenic sarcoma cell, a renal carcinoma cell, a hepatocellular carcinoma cell, a bladder cancer cell, a gastric carcinoma cell, a head and neck squamous carcinoma cell, a melanoma cell and a leukemia cell. In some embodiments, a cell that is targeted in a method described herein is a hyperproliferative and/or hyperplastic cell. In some embodiments, a cell that is targeted in a method described herein is a dysplastic cell. In yet another embodiment, a cell that is targeted in a method described herein is a metastatic cell.

In some embodiments of any of the methods, the method further comprises additional treatment steps. For example, in some embodiments, the method further comprises a step wherein a targeted cell and/or tissue (e.g., a cancer cell) is exposed to radiation treatment or a second therapeutic agent (e.g., chemotherapeutic agent). For example, methods are provided for treating or preventing cancer comprising administering (i) a purified anti-c-met antibody (e.g., onartuzumab) composition and/or anti-c-met antibody purified by the methods described herein and (ii) a second therapeutic agent. In some embodiments, the second therapeutic agent is an EGFR inhibitor (e.g., erlotinib), VEGF inhibitor (e.g., bevacizumab), or taxane (e.g., paclitaxel).

In some embodiments of any of the methods described herein, the method further comprises administering an effective amount of a second therapeutic agent. In some embodiments, the dose of anti-c-met antibody is about 15 mg/kg. In some embodiments, the dose of anti-c-met antibody is about 10 mg/kg.

In some embodiments, the second therapeutic agent is an EGFR inhibitor. In some embodiments, the EGFR inhibitor is erlotinib (N-(3-ethynylphenyl)-6,7-bis(2-methoxyethoxy)-4-quinazolinamine) In some embodiments, the dose of anti-c-met antibody is about 15 mg/kg administered day one of a 21 day cycle. For example, provided are methods of treating cancer (e.g., NSCLC) comprising administering (i) a purified anti-c-met antibody (e.g., onartuzumab) composition and/or anti-c-met antibody purified by the methods described herein, wherein the anti-c-met antibody is administered at a dose of 15 mg/kg every three weeks; and (ii) erlotinib (N-(3-ethynylphenyl)-6,7-bis(2-methoxyethoxy)-4-quinazolinamine), wherein erlotinib is administered at a dose of 150 mg, each day of a three week cycle.

In some embodiments, the second therapeutic agent is a taxane (e.g., paclitaxel). In some embodiments, the cancer is breast cancer. In some embodiments, the breast cancer is an ER-negative, PR-negative, and HER2-negative (ER-, PR-, and HER2-; or triple-negative) metastatic breast cancer. In some embodiments, the dose of anti-c-met antibody is about 10 mg/kg. on day 1 and day 15 of a 28-day cycle. For example, provided are methods for treating cancer (e.g., breast cancer) comprising administering (i) a purified anti-c-met antibody (e.g., onartuzumab) composition and/or anti-c-met antibody purified by the methods described herein, wherein the anti-c-met antibody is administered at a dose of 10 mg/kg on day 1 and day 15 of a 28-day cycle; and (ii) paclitaxel, wherein paclitaxel is administered at a dose of 90 mg/m² by IV infusion on day 1, day 8, and day 15 of the 28-day cycle. In some embodiments, the method increases survival of the patient, decreases the patient's risk of cancer recurrence and/or to increases the patient's likelihood of survival. In some embodiments, the method further comprises administration of an anti-VEGF antibody (e.g., bevacizumab). For example, provided are methods for treating cancer (e.g., breast cancer) comprising administering (i) a purified anti-c-met antibody (e.g., onartuzumab) composition and/or anti-c-met antibody purified by the methods described herein, wherein the anti-c-met antibody is administered at a dose of 10 mg/kg on day 1 and day 15 of a 28-day cycle; (ii) an anti-VEGF antibody (e.g., bevacizumab), wherein the anti-VEGF antibody is administered at a dose of 10 mg/kg on Day 1 and Day 15 of the 28-day cycle; and (iii) paclitaxel, wherein paclitaxel is administered at a dose of 90 mg/m² by IV infusion on Day 1, Day 8, and Day 15 of the 28-day cycle.

A purified anti-c-met antibody (e.g., onartuzumab) composition and/or anti-c-met antibody purified by the methods described herein can be used either alone or in combination with other agents in a therapy. For instance, a purified anti-c-met antibody (e.g., onartuzumab) composition and/or anti-c-met antibody purified by the methods described herein may be co-administered with a second therapeutic agent (e.g., another antibody, chemotherapeutic agent(s) (including cocktails of chemotherapeutic agents), other cytotoxic agent(s), anti-angiogenic agent(s), cytokines, and/or growth inhibitory agent(s)). In some embodiments, the second therapeutic agent is administered concurrently or sequentially. The second therapeutic agent can be administered separately from the purified anti-c-met antibody (e.g., onartuzumab) composition and/or anti-c-met antibody purified by the methods, but as a part of the same treatment regimen. Where the purified anti-c-met antibody (e.g., onartuzumab) composition and/or anti-c-met antibody purified by the methods described herein inhibit tumor growth, it may be particularly desirable to combine it with one or more other therapeutic agent(s) which also inhibits tumor growth. For instance, purified anti-c-met antibody (e.g., onartuzumab) composition and/or anti-c-met antibody purified by the methods described herein may be combined with an EGFR inhibitor, an anti-VEGF antibody and/or anti-ErbB antibodies in a treatment scheme, e.g. in treating any of the diseases described herein, including colorectal cancer, metastatic breast cancer and kidney cancer.

Such combined therapies noted above encompass combined administration (where two or more agents are included in the same or separate formulations), simultaneously, and separate administration, in which case, administration of the pharmaceutical formulation can occur prior to, and/or following, administration of the additional therapeutic agent and/or adjuvant.

Accordingly, in some embodiments of any of the methods described herein, the method comprises targeting a cell wherein c-met or hepatocyte growth factor, or both, is more abundantly expressed by said cell (e.g., a cancer cell) as compared to a normal cell of the same tissue origin. A c-met-expressing cell can be regulated by HGF from a variety of sources, i.e. in an autocrine or paracrine manner. C-met activation and/or signaling can also occur independent of ligand. Hence, in some embodiments, c-met activation in a targeted cell occurs independent of ligand.

The purified anti-c-met antibody (e.g., onartuzumab) composition and/or anti-c-met antibody purified by the methods described herein can be administered to a human subject for therapeutic purposes. Moreover, purified anti-c-met antibody (e.g., onartuzumab) composition and/or anti-c-met antibody purified by the methods described herein can be administered to a non-human mammal expressing an antigen with which the immunoglobulin cross-reacts (e.g., a primate, pig or mouse) for veterinary purposes or as an animal model of human disease.

The purified anti-c-met antibody (e.g., onartuzumab) composition and/or anti-c-met antibody purified by the methods described herein can be used to treat, inhibit, delay progression of, prevent/delay recurrence of, ameliorate, or prevent diseases, disorders or conditions associated with abnormal expression and/or activity of one or more antigen molecules, including but not limited to malignant and benign tumors; non-leukemias and lymphoid malignancies; neuronal, glial, astrocytal, hypothalamic and other glandular, macrophagal, epithelial, stromal and blastocoelic disorders; and inflammatory, angiogenic and immunologic disorders.

In some embodiments of any of the methods, an immunoconjugate comprising the purified anti-c-met antibody (e.g., onartuzumab) composition and/or anti-c-met antibody purified by the methods described herein conjugated with a cytotoxic agent is administered to the patient. In some embodiments, the immunoconjugate and/or antigen to which it is bound is/are internalized by the cell, resulting in increased therapeutic efficacy of the immunoconjugate in killing the target cell to which it binds. In some embodiments, the cytotoxic agent targets or interferes with nucleic acid in the target cell.

The purified anti-c-met antibody (e.g., onartuzumab) composition and/or anti-c-met antibody purified by the methods described herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. In some embodiments, the antibody is administered intravenously. Dosing can be by any suitable route, e.g. by injections, such as intravenous or subcutaneous injections, depending in part on whether the administration is brief or chronic. Various dosing schedules including, but not limited to, single or multiple administrations over various time-points, bolus administration, and pulse infusion are contemplated herein.

Purified anti-c-met antibody (e.g., onartuzumab) composition and/or anti-c-met antibody purified by the methods described herein are dosed and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The purified anti-c-met antibody (e.g., onartuzumab) composition and/or anti-c-met antibody purified by the methods described herein need not be, but is optionally formulated with one or more agents currently used to prevent or treat the disorder in question. The effective amount of such other agents depends on the amount of antibodies of in the formulation, the type of disorder or treatment, and other factors discussed above. These are generally used in the same dosages and with administration routes as used hereinbefore or about from 1 to 99% of the heretofore employed dosages described herein, or any dosage and by any route that is empirically/clinically determined to be appropriate.

For the prevention or treatment of disease, the appropriate dosage of the purified anti-c-met antibody (e.g., onartuzumab) composition and/or anti-c-met antibody purified by the methods described herein (when used alone or in combination with one or more additional therapeutic agents) will depend on the type of disease to be treated, the type of antibody, the severity and course of the disease, whether the purified anti-c-met antibody (e.g., onartuzumab) composition and/or anti-c-met antibody purified by the methods described herein is administered for preventive or therapeutic purposes, previous therapy, the patient's clinical history and response to the anti-c-met antibody, and the discretion of the attending physician. The purified anti-c-met antibody (e.g., onartuzumab) composition and/or anti-c-met antibody purified by the methods described herein are suitably administered to the patient at one time or over a series of treatments. Depending on the type and severity of the disease, about 10 mg/kg, about 15 mg/kg or greater (e.g., 15-20 mg/kg) dosage of the anti-c-met antibody is administered to the patient, whether, for example, by one or more separate administrations, or by continuous infusion. In some embodiments, the dose of anti-c-met antibody is about 15 mg/kg. In some embodiments, the dose of anti-c-met antibody is about 15 mg/kg administered day one of a 21 day cycle. In some embodiments, the dose of anti-c-met antibody is about 10 mg/kg. In some embodiments, the dose of anti-c-met antibody is about 10 mg/kg administered on day 1 and 15 of a 28 day cycle.

Doses may be administered intermittently, e.g. about any of every week, every two weeks, every three weeks, or every four weeks.

For repeated administrations over several days or longer, depending on the condition, the treatment would generally be sustained until a desired suppression of disease symptoms occurs. However, other dosage regimens may be useful. The progress of this therapy is easily monitored by conventional techniques and assays.

### VII. ARTICLES OF MANUFACTURE

Article of manufacture comprising the purified anti-c-met antibody (e.g., onartuzumab) composition, pharmaceutical formulations comprising the purified anti-c-met antibody composition, and/or anti-c-met antibody purified by the methods described herein and use thereof for the treatment, prevention and/or diagnosis of the disorders are provided. The article of manufacture comprises a container and a label or package insert on or associated with the container. Suitable containers include, for example, bottles, vials, syringes, IV solution bags etc. The containers may be formed from a variety of materials such as glass or plastic. The container holds the purified anti-c-met antibody (e.g., onartuzumab) composition and/or anti-c-met antibody purified by the methods described herein which is by itself or when combined with another composition effective for treating, preventing and/or diagnosing the condition and may have a sterile access port (for example the container may be an intravenous solution bag or a vial having a stopper pierceable by a hypodermic injection needle). For example, provided herein are articles of manufacture and kits comprising a container with a purified anti-c-met antibody (e.g., onartuzumab) composition and/or anti-c-met antibody purified by the methods described herein. The label or package insert indicates that the composition is used for treating the condition of choice, such as cancer. In some embodiments, the cancer is non-small cell lung cancer (NSCLC), glioblastoma, pancreatic cancer, sarcoma, renal cell carcinoma, gastric cancer, colorectal cancer, or breast cancer. In some embodiments, the cancer is stage IIIb and/or stage IV cancer. In some embodiments, the cancer is locally advanced or metastatic cancer. In some embodiments, the therapy is second line or third line therapy (e.g., second line or third line NSCLC therapy). In some embodiments, the cancer is EGFR mutant. In some embodiments, the cancer is EGFR wild-type. In some embodiments, the cancer is c-met positive (expresses high levels of c-met, for example, by immunohistochemistry). In some embodiments, the dose of anti-c-met antibody is about 15 mg/kg. In some embodiments, the dose of anti-c-met antibody is about 15 mg/kg administered day one of a 21 day cycle. In some embodiments, the dose of anti-c-met antibody is about 10 mg/kg. In some embodiments, the dose of anti-c-met antibody is about 10 mg/kg administered on day 1 and 15 of a 28 day cycle.

Provided are methods of packaging an article of manufacture comprising adding a composition comprising an anti-c-met antibody and/or pharmaceutical formulation comprising the purified anti-c-met antibody composition, wherein HCP in the composition and/or pharmaceutical formulation is less than or equal to about 50 ng/mg. Further, provided are methods of packaging an article of manufacture comprising adding a lot (e.g., batch) of composition comprising an anti-c-met antibody and/or lot (e.g., batch) of pharmaceutical formulation comprising the purified anti-c-met antibody composition, wherein average HCP in the lot is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.3, about 8.4, or about 8.5. In some embodiments, the anti-c-met antibody is onartuzumab.

Provided are also containers (e.g., vials) comprising compositions comprising an anti-c-met antibody and/or pharmaceutical formulations comprising the anti-c-met antibody composition, wherein HCP in the composition or pharmaceutical formulation is present in the composition in less than or equal to about 50 ng/mg. Also provided are also containers (e.g., vials) comprising a lot (e.g., batch) of compositions comprising an anti-c-met antibody and/or a lot (e.g., batch) of pharmaceutical formulations comprising the anti-c-met antibody composition, wherein average HCP in the lot is less than or equal to about 50 ng/mg. In some embodiments, the HCP and/or average HCP is less than or equal to about any of 34 ng/mg, 30 ng/mg, 25 ng/mg, 20 ng/mg, 19 ng/mg, 18 ng/mg, 17 ng/mg, 16 ng/mg, 15 ng/mg, 14 ng/mg, 13 ng/mg, 12 ng/mg, 11 ng/mg, 10 ng/mg, or 9 ng/mg. In some embodiments, the HCP and/or average HCP is between about any of 5 ng/mg and 20 ng/mg, 5 ng/mg and 25 ng/mg, 5 ng/mg and 15 ng/mg, 1 ng/mg and 30 ng/mg, 1 ng/mg and 25 ng/mg, 1 ng/mg and 20 ng/mg, 1 ng/mg and 15 ng/mg, or 1 ng/mg and 10 ng/mg. In some embodiments, the HCP and/or average HCP is about any of 5, 5.5, 6.5, 7, 7.5, 8, 8.5, 9, 9.5, 10, 10.5, 11, 11.5, 12, 12.5, 13, 13.5, 14, 14.5, 15, 15.5, 16, 16.5, 17, or 17.5 ng/mg. In some embodiments, the anti-c-met antibody is produced in E. coli. In some embodiments, the HCP and/or average HCP is ECP and/or average ECP. In some embodiments, the anti-c-met antibody is an antibody described in Section IV. In some embodiments, the anti-c-met antibody is about 100 kDa. In some embodiments, the anti-c-met antibody has a pI of about 8.3, about 8.4, or about 8.5. In some embodiments, the anti-c-met antibody is onartuzumab.

The article of manufacture in this embodiment may further comprise a package insert indicating that the first and second antibody compositions can be used to treat a particular condition, e.g. cancer. In some embodiments, the cancer is non-small cell lung cancer (NSCLC), glioblastoma, pancreatic cancer, sarcoma, renal cell carcinoma, gastric cancer, colorectal cancer, or breast cancer. In some embodiments, the cancer is stage IIIb and/or stage IV. In some embodiments, the cancer is locally advanced or metastatic cancer. In some embodiments, the therapy is second line or third line therapy (e.g., second line or third line NSCLC therapy). In some embodiments, the cancer is EGFR mutant. In some embodiments, the cancer is EGFR wild-type. In some embodiments, the cancer is c-met positive (expresses high levels of c-met, for example, by immunohistochemistry). In some embodiments, the dose of anti-c-met antibody is about 15 mg/kg. In some embodiments, the dose of anti-c-met antibody is about 15 mg/kg administered day one of a 21 day cycle.

Alternatively, or additionally, in some embodiments of any of the articles of manufacture, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Moreover, the article of manufacture may comprise (a) a first container with a purified anti-c-met antibody (e.g., onartuzumab) composition and/or anti-c-met antibody purified by the methods described herein contained therein; and (b) a second container with a composition contained therein, wherein the composition comprises a further cytotoxic agent.

In some embodiments, the second therapeutic agent is an EGFR inhibitor. In some embodiments, the EGFR inhibitor is erlotinib (N-(3-ethynylphenyl)-6,7-bis(2-methoxyethoxy)-4-quinazolinamine) In some embodiments, the article of manufacture comprises instructions for administration of about 15 mg/kg administered day one of a 21 day cycle of anti-c-met antibody formulation and 150 mg, each day of a three week cycle of erlotinib. In some embodiments, the article of manufacture comprises instructions for the treatment of cancer (e.g., NSCLC).

In some embodiments, the second therapeutic agent is a taxane (e.g., paclitaxel). In some embodiments, the article of manufacture comprises instructions for administration of about 10 mg/kg. on day 1 and day 15 of a 28-day cycle of the anti-c-met antibody formulation and 90 mg/m² by IV infusion on day 1, day 8, and day 15 of the 28-day cycle of paclitaxel. In some embodiments, the article of manufacture comprises a third container with a composition contained therein, wherein the composition comprises a third therapeutic agent, wherein the third therapeutic agent is an anti-VEGF antibody (e.g., bevacizumab). In some embodiments, the article of manufacture comprises instructions for administration of about 10 mg/kg. on day 1 and day 15 of a 28-day cycle of the anti-c-met antibody formulation, 90 mg/m² by IV infusion on day 1, day 8, and day 15 of the 28-day cycle of paclitaxel, and 10 mg/kg on Day 1 and Day 15 of the 28-day cycle of the anti-VEGF antibody (e.g., bevacizumab). In some embodiments, the article of manufacture comprises instructions for the treatment of cancer. In some embodiments, the cancer is breast cancer (e.g., ER-negative, PR-negative, and HER2-negative (ER-, PR-, and HER2-; or triple-negative) metastatic breast cancer). In some embodiments, the method increases survival of the patient, decreases the patient's risk of cancer recurrence and/or to increases the patient's likelihood of survival.

It is understood that any of the above articles of manufacture may include an immunoconjugate of the purified anti-c-met antibody (e.g., onartuzumab) composition and/or anti-c-met antibody purified by the methods described herein in place or in addition to the anti-c-met antibody.

Further provided herein are methods of making any of the articles of manufacture described herein.

The following are examples of the purified anti-c-met antibody (e.g., onartuzumab) composition and/or methods of purifying antic-met-antibodies. It is understood that various other embodiments may be practiced, given the general description provided above.

### EXAMPLES

### Examples

### Onartuzumab Purification Process

Materials and Methods

E. Coli Protein (ECP) Level Assay

A sandwich ELISA was used to detect and quantify E. coli proteins (ECPs) when present in product samples. Affinity-purified antibodies specific to ECPs were immobilized onto microtiter plate wells. ECPs, if present in the sample, bind to the coated antibody. Bound ECPs were detected with anti-ECP conjugated to horseradish peroxidase (HRP), which reacts with substrate 3,3′,5,5′-tetramethylbenzidine (TMB) and produces a colorimetric signal. The anti-ECP reagents were developed in-house against a complex mixture of E coli proteins. A five-parameter curve-fitting program was used to generate a standard curve, and sample concentrations are extrapolated from the standard curve.

DNA Level Assay

To detect and quantify E. coli DNA in product samples, DNA from samples was extracted and subjected to TaqMan real-time polymerase chain reaction (PCR) using PCR primers and probe. The amplicons (amplified product) were quantified in direct proportion to the increase in fluorescence emission measured continuously during the DNA amplification. A standard curve was used to quantify the amount of E. coli DNA in the sample.

LpA Level Assay

This test procedure was performed using a sandwich ELISA to detect and quantify protein A when present in product samples. Chicken anti-staphylococcal protein A antibody as immobilized on microtiter wells. Samples, standards, and controls were pre-treated before incubation in the wells, where the protein A binds to the coated antibody. The bound protein A was detected with chicken anti-protein A conjugated to HRP, which reacts with substrate 3,3′,5,5′-TMB and produces a colorimetric signal. This pre-treatment was based on the dissociation of protein A from the protein A/IgG complex, making protein A fully accessible to its detection reagents (Zhu-Shimoni et al., J. Immunol. Methods 341:59-67 (2009). Thus it allowed the protein A to be detected without interference from excess product molecules in the sample. Specific ligand (e.g., ProSep-vA or MabSelect SuRe™) corresponding to the ligand immobilized on the protein A column was used as the standard in the assay. A five-parameter curve-fitting program was used to generate a standard curve, and sample concentrations were extrapolated from the standard curve.

LAL Level Assay

Bacterial endotoxins are lipopolysaccharide (LPS) components of the cell walls of gram-negative bacteria that can be released by destruction of the microbial cell or by shedding from live cells. The kinetic chromogenic method was used for the detection and quantification of bacterial endotoxins by Limulus Amebocyte Lysate (LAL). This assay was qualified according to USP and Ph. Eur. requirements.

The kinetic chromogenic method was based on the activation of a proenzyme in the LAL reagent by the presence of bacterial endotoxin. Upon activation, the enzyme catalyses the cleavage of a chromophore, producing a yellow color that was quantified spectrophotometrically. The rate of color change was directly proportional to the amount of endotoxin present and the reaction time. A standard curve was generated from the log/log correlation between the endotoxin concentration and the reaction time needed to produce a significant amount of color.

Monomer, Fragment and Aggregate Assay

Size-exclusion chromatography was used to monitor the size heterogeneity of onartuzumab under native conditions by employing the TSK-GEL G3000SW_{XL} column to separate onartuzumab high-molecular-weight species (aggregates), main peak (monomer), and low-molecular-weight species (fragments).

Main Peak, Acidic Variant, and Basic Variant Assay

Cation-exchange chromatography was used to quantitatively monitor charge heterogeneity by employing the Dionex ProPac weak cation-exchange column to separate onartuzumab into an acidic region, a main peak, and a basic region.

Results

Onartuzumab is a one-armed, monovalent anti-c-met antibody currently produced in Escherichia coli (E. coli). Given the need to minimize aggregation of monovalent antibodies (formation of multimer and oligomers), to maintain monovalent structure (rather than formation of an agonist bivalent antibody with two heavy chain and two light chains), and/or due to the very similar electrostatic properties of onartuzumab and host cell impurities/contaminants, multiple onartuzumab purification processes were pursued as detailed in Table 2.

TABLE 2
Onartuzumab Purification Process.

| | Process A | Process B | Process C | Process D | Process E | Process F |
|---|---|---|---|---|---|---|
| Extraction | Cell Paste | Homogenization | Homogenization | Homogenization | Homogenization | Homogenization |
| | Resuspension | | | | | |
| | Homogenization | | | | | |
| | Cationic | Cationic | Cationic | Cationic | Cationic | Cationic |
| | Polymer | Polymer/ | Polymer/ | Polymer/ | Polymer/ | Polymer/ |
| | | Dilution | Dilution | Dilution | Dilution | Dilution |
| | | | | | Flocculation | Flocculation |
| | | | | | Step/Dilution | Step/Dilution |
| | Centrifugation | Centrifugation | Centrifugation | Centrifugation | Centrifugation | Centrifugation |
| Chrom 1 | Protein A | Protein A | Protein A | Protein A | Protein A | Protein A |
| | Resin 1 | Resin 1 | Resin 1 | Resin 1 | Resin 2 | Resin 2 |
| Chrom 2 | Strong Cation | Weak CE | Weak CE | Weak CE | Weak Anion | Weak AE |
| | Exchange (CE) | | | | Exchange (AE) | |
| Chrom 3 | N/A | Strong CE | Strong CE | Strong CE | Strong CE | Strong CE |
| Final | HIC | HIC | Strong AE | Strong AE | Strong AE | Strong AE |
| Chrom | Resin 1 | Resin 2 | | | | |
| Buffer | UFDF | UFDF | UFDF | UFDF | UFDF | UFDF |
| Exchange | | | | | | |

The processes as described above yielded batches of compositions comprising onartuzumab with the attributes as described in Table 3.

TABLE 3

| | Process A | Process B | Process C | Process D | Process E | Process F |
|---|---|---|---|---|---|---|
| ECP (ng/mg) | 435 | 150 | 33-34 | 17-33 | 7-15 | 6-10 |
| DNA (pg/mg) | | <0.3 | <0.3 | <0.1-<0.3 | <0.2-<0.3 | |
| LpA (ng/mg) | | <2 | <2 | <2 | <2 | |
| LAL (EU/mg) | 0.04 | 0.01 | <0.002-0.001 | <0.001-0.005 | <0.007 | |
| Aggregates (%) | 0.1 | 0.3 | 0.2 | 0.2-0.3 | 0.1-0.2 | |
| Monomer (%) | 99 | 99.2-99.3 | 99.5-99.7 | 99.6-99.7 | 99.8-99.9 | |
| Fragment (%) | <1 | 0.4-0.5 | 0.1-0.3 | 0.1-0.2 | 0.0 | |
| Acidic Variant (%) | | 23-24 | 13.8-16.1 | 11.0-12.4 | 15.9-19.9 | |
| Main Peak (%) | | 73 | 82.0-84.6 | 85.8-86.3 | 78.9-83.4 | |
| Basic Variant (%) | | 3 | 1.1-2.0 | 1.8-2.0 | 0.5-1.3 | |

In comparing Process A and Process B, the differences resulted in a significant improvement in purification process and/or purity of the composition comprising onartuzumab observed as outlined in Table 4.

TABLE 4

| | | | Process Differences | |
|---|---|---|---|---|
| | Process A | Process B | Process A/Process B | Results |
| Extraction | Cell paste | Homogenization | (1) Eliminated cell paste | (1) Step elimination; faster |
| | resuspension | | collection & Resuspension | processing |
| | Homogenization | | | |
| | Cationic Polymer | Cationic Polymer/ | (1) Increased PEI | (1) Improved clarification |
| | | Dilution | concentration from 0.2% | |
| | | | to 0.4% | |
| | Centrifugation | Centrifugation | N/A | N/A |
| Chrom 1 | Protein A | Protein A | (1) Removed hazardous | (1) Reduced environmental |
| | Resin 1 | Resin 1 | waste component | impact; (2) smaller pool |
| | | | TMAC from wash 2 | volume |
| | | | buffer, (2) Changed end- | |
| | | | pool criteria | |
| Chrom 2 | Strong CE | Weak CE | (1) Changed resin from | (1) Enhanced ECP removal |
| | | | strong CE to weak CE; | and resin cleaning, greater |
| | | | (2) changed column | binding capacity for |
| | | | elution from gradient to | residual PEI; (2) improved |
| | | | step (3) changed pooling | process robustness and |
| | | | criteria | efficiency (3) reduced |
| | | | | aggregates |
| Chrom 3 | N/A | Strong CE | (1) Added a strong CE | (1) Enhanced ECP removal |
| | | | step | |
| Final | HIC | HIC | (1) Changed HIC resin | (1) Enhanced ECP removal |
| Chrom | Resin 1 | Resin 2 | to Phenyl Sepharose FF | and yield |
| | | | HiSub; changed | |
| | | | operation from bind & | |
| | | | elute to flow-through | |
| Buffer | UFDF | UFDF | N/A | N/A |
| Exchange | | | | |

As noted in Table 4, one difference in the purification of Process A compared to Process B was a change of chromatography step 2 (Chrom 2) from a strong CE column to a weak CE column. In developing Process B, potential CE resins were evaluated. A CE resin screen was performed using CM Sepharose FF (weak CE resin), SP Sepharose FF (strong CE resin), and SP XL resins (strong CE resin). The weak CE resin demonstrated better ECP clearance compared to SP Sepharose FF (strong CE resin) as shown in Table 5. Also, weak CE resin could be regenerated back to its original appearance while the other resins were left with a brownish color after base regeneration. Further, when pooling 0.5-0.5 OD, the last fractions from the weak CE resin and strong CE resin (SP Sepharose FF) runs have 50% aggregate. In contrast, when pooling 1-1 OD, this aggregate was removed from the pool and an aggregate level of less than 1% was seen in these pools without affecting the product yield significantly.

TABLE 5
Resin screen conditions:
Equil/wash: 25 mM MES, pH 6.5
Load: Pro A pool, pH 5.0
Elution: 15CV, 0-140 mM NaCl, 25 mM MES, pH 6.5
pool from 0.5-0.5 OD
Capacity: 20 g/l

| | CM Seph FF | | | SP Seph FF | | |
|---|---|---|---|---|---|---|
| | % | ECP, | % | % | ECP, | % |
| Load condition | rec | ppm | agg | rec | ppm | agg |
| pH 5, 2.8 mS/cm | 83% | 824 | 13.4% | 88% | 776 | 10.3% |
| pH 5, 5.5 mS/cm | 98% | 662 | 12.4% | 92% | 2171 | 12.9% |
| pH 5.5, 2.9 mS/cm | 86% | 652 | 13.5% | 91% | 907 | 15.0% |
| pH 6.5, 1.1 mS/cm | 99% | 624 | 13.1% | 95% | 856 | 15.9% |
| pH 6.5, 4.4 mS/cm | 94% | 464 | 12.9% | 93% | 892 | 6.0% |

| | SPXL | | |
|---|---|---|---|
| Load condition | % rec | ECP, ppm | % agg |
| pH 5, 2.8 mS/cm | 76% | 737 | 1.1% |
| pH 5, 5.5 mS/cm | 80% | 662 | 0.9% |
| pH 5.5, 2.9 mS/cm | 85% | 655 | 0.9% |
| pH 6.5, 1.1 mS/cm | 80% | 658 | 1.0% |
| pH 6.5, 4.4 mS/cm | 42% | 356 | 1.2% |

% rec = % recovery; % agg = % aggregates

Further, in developing Process B, potential hydrophobic interactive chromatography (HIC) resins were evaluated for the final chromatography step. As shown in Table 6, HIC resins, Phenyl Sepharose FF HiSub from GE Health Science (Resin 1), Toyopearl Phenyl-650M from TOSOH (Resin 2), Toyopearl Hexyl-650C from TOSOH (Resin 3), and Toyopearl Butyl-650M from TOSOH (Resin 4), were evaluated via the AKTA scouting method and processed using the following run conditions: mode: flowthrough, pH 7.0, flow rate: 150 cm/hr, and max load density: 50 mg/ml. The resin was equilibration in 5 column volumes (CV) of buffer (0.3 M Na₂SO₄, 50 mM Na₃PO₄, pH 7.0). The sample, conditioned SP Sepharose XL pool (conditioned 1:1 with 0.6 M Na₂SO₄, 0.1 M Na₃PO₄, pH 7.0 buffer; starting pool criteria: 0.5 OD), was loaded onto the column, and the protein of interest (onartuzumab) was eluted using 15-20 CV of buffer (0.3 M Na₂SO₄, 50 mM Na₃PO₄, pH 7.0) with ending pool criteria of 0.5 OD.

Based on the results as shown in Table 6, the HIC resin, Phenyl Sepharose HiSub, had the best overall performance by achieving a step yield of 82% vs. 70% with HiPropyl (data not shown) and impurity clearance of 121 ppm ECP and 1.4% aggregates.

TABLE 6

| | | | | | Elution | | % |
|---|---|---|---|---|---|---|---|
| | Vol. | Prot. | Yield | Pre-Pool | Wash | ECP | Aggre- |
| Sample | (ml) | (mg/ml) | (%) | Vol (CV) | Vol (CV) | (ppm) | gates |
| Load (Cond. SP-XL Pool) | 12.0 | 3.26 | — | — | — | 152 | 2.6 |
| Pools | | | | | | | |
| Resin 1: Phenyl Sepharose HiSub | 21.2 | 1.51 | 82 | 10.0 | 20.0 | 121 | 1.4 |
| Resin 2: Toyopearl Phenyl | 19.4 | 1.98 | 98 | 7.13 | 15.0 | 164 | 1.8 |
| Resin 3: Toyopearl Hexyl | 20.9 | 1.59 | 85 | 8.70 | 18.4 | 149 | 0.9 |
| Resin 4: Toyopearl Butyl | 17.9 | 1.71 | 78 | 8.64 | 16.8 | 181 | 1.3 |

In comparing Process B and Process C, the differences resulted in a significant improvement in purification process and/or purity of the composition comprising onartuzumab observed as outlined in Table 7.

TABLE 7

| | | | Process Differences | |
|---|---|---|---|---|
| | Process B | Process C | Process B/Process C | Results |
| Extraction | Homogenization | Homogenization | N/A | N/A |
| | Cationic Polymer/ | Cationic Polymer/ | N/A | N/A |
| | Dilution | Dilution | | |
| | Centrifugation | Centrifugation | N/A | N/A |
| Chrom 1 | Protein A | Protein A | N/A | N/A |
| | Resin 1 | Resin 1 | | |
| Chrom 2 | Weak CE | Weak CE | (1) Increased wash and | (1) Improved |
| | | | elution buffer pH from | process robustness |
| | | | 6.5 to 7.1; changed buffer | and efficiency |
| | | | components from MES to | |
| | | | MOPS | |
| Chrom 3 | Strong CE | Strong CE | (1) Load pH increased | (1) Enhanced ECP |
| | | | from 6.5 to 7.0 | removal |
| Final | HIC | Strong AE | (1) Changed resin from | (1) Enhanced ECP |
| Chrom | Resin 2 | | HIC Resin 2 to strong | removal and |
| | | | AE; changed operation | improved process |
| | | | from flow-through to bind | robustness and |
| | | | & elute | efficiency |
| Buffer | UFDF | UFDF | N/A | N/A |
| Exchange | | | | |

In developing Process C, to eliminate the required pH adjustment of strong CE resin load, the buffers used in the weak CE and strong CE columns were changed from MES to MOPS. This also had an advantage facilitating ease of processing. Table 8 below shows a comparison of MOPS and MES with further purification on the weak CE resin resulted in similar ECP values. Comparable results were seen when changing from Process B conditions (25 mM MES, 60 mM NaOAc pH 6.5) to 25 mM MOPS, 50 mM NaOAc pH 7.1.

TABLE 8

| | | | CMFF | |
|---|---|---|---|---|
| | | | | ECP |
| Load | Equil | Elute | % Yield | ppm |
| 1K-10 | 25 mM MES, | 25 mM MES, 60 mM | 83% | 805 |
| | pH 6.5 | NaOAc, pH 6.5 | | |
| 1K-10 | 25 mM MOPS, | 25 mM MOPS, 50 mM | 82% | 794 |
| | pH 7.1 | NaOAc, pH 7.1 | | |

In addition, when running the strong CE resin with load at either pH 6.5 or 7.0, the higher pH load appeared to give better ECP clearance as shown Table 9. Further, the yields were comparable as shown in Table 9.

TABLE 9

| Load pH | ECP (ppm) | Yield (%) | Aggregate (%) |
|---|---|---|---|
| 6.5 | 368 | 78 | 0.5 |
| 7.0 | 281 | 79 | 0.6 |

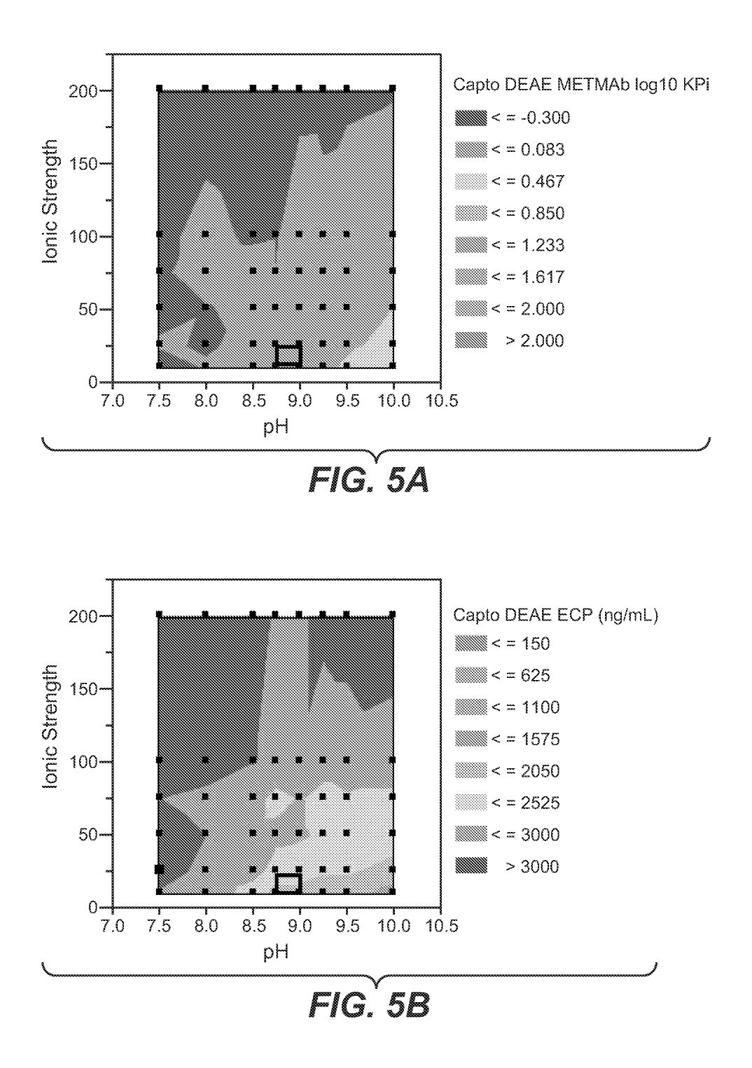

The strong AE resin (Q Sepharose FF) run under the gradient elution conditions as shown in FIG. 4 resulted in good resolution of ECP and aggregate. The chromatogram in FIG. 4 includes traces for ECP in ng/mL and % aggregate (Note that OA5D5 in FIG. 4 is onartuzumab). The distribution of the ECP and the aggregate indicated that the strong AE resin would adequately remove ECP and could replace the HIC resin as the final chromatography step. See also Table 10.

TABLE 10

| | Onartuzumab | Volume | Onartuzumab | Yield | ECP | Aggregation |
|---|---|---|---|---|---|---|
| Sample | (mg/mL) | (mL) | (mg) | (%) | (ppm) | (%) |
| Strong AE Load | 1.2 | 892 | 1087 | NA | 571 | 0.9 |
| (Weak CE Pool) | | | | | | |
| Strong AE Pool | 2.3 | 404 | 922 | 85 | 91 | 0.4 |
| 0 to 1 OD | | | | | | |

The following conditions were studied to determine if the parameters and operating range of the strong AE resin could be run without affecting product purity and recovery. Runs were done with 40 mM, 45 mM and 50 mM NaCl in the elution buffer. The pH of the elution buffer was tested at 8.7, 8.9 and 9.2. The salt concentration of the wash buffer was tested at 10 mM, 25 mM and 30 mM NaCl. The effect of under-loading the strong AE column was also tested by a run with a 15 g/L load density. All runs proved the robustness of the final strong AE resin operating conditions as shown in Table 11.

TABLE 11

| | | Cond. | Yield | ECP | Aggregate |
|---|---|---|---|---|---|
| | pH | (mS/cm) | (%) | (ppm) | (%) |
| Cond. Elution Buffer | | | | | |
| 40 mM NaCl | 8.9 | 5.0 | 75 | 24 | 0.2 |
| 45 mM NaCl | 8.9 | 5.5 | 82 | 36 | 0.5 |
| 50 mM NaCl | 8.9 | 6.1 | 85 | 65 | 1.6 |
| pH Elution Buffer | | | | | |
| 45 mM NaCl, | 8.7 | 5.8 | 92 | 54 | 1.0 |
| 45 mM NaCl | 8.9 | 5.5 | 82 | 36 | 0.5 |
| 45 mM NaCl | 9.2 | 5.5 | 62 | 39 | 0.8 |
| Cond. Wash Buffer | | | | | |
| 10 mM NaCl | 9.1 | 1.5 | 78 | 45 | 0.4 |
| 25 mM NaCl | 9.1 | 3.2 | 82 | 36 | 0.5 |
| 30 mM NaCl | 9.1 | 3.7 | 85 | 35 | 0.6 |
| Loading Density | | | | | |
| 30 g/L | — | — | 75 | 27 | 0.2 |
| 15 g/L resin | — | — | 78 | 18 | 0.2 |

In comparing Process C and Process D, the differences resulted in a significant improvement in purification process and/or purity of the composition comprising onartuzumab observed as outlined in Table 12.

TABLE 12

| | | | Process Differences | |
|---|---|---|---|---|
| | Process C | Process D | Process C/Process D | Results |
| Extraction | Homogenization | Homogenization | N/A | N/A |
| | Cationic Polymer/ | Cationic Polymer/ | (1) Increased | (1) Improved |
| | Dilution | Dilution | dilution by 10% | product recovery |
| | Centrifugation | Centrifugation | N/A | N/A |
| Chrom 1 | Protein A | Protein A | N/A | N/A |
| | Resin 1 | Resin 1 | | |
| Chrom 2 | Weak CE | Weak CE | N/A | N/A |
| Chrom 3 | Strong CE | Strong CE | N/A | N/A |
| Final Chrom | Strong AE | Strong AE | N/A | N/A |
| Buffer Exchange | UFDF | UFDF | N/A | N/A |

In comparison to Process C, the protein A pool product recovery of Process D was increased approximately 10% when utilizing a 10% increase in dilution prior to centrifugation (average protein A pool mass (normalized): Process C-1X and Process D-1.1X). In this example, the net improvement in product recovery over the centrifugation step translated downstream to a net increase in product recovery over protein A.

In comparing Process D and Process E, the differences resulted in a significant improvement in purification process and/or purity of the composition comprising onartuzumab observed as outlined in Table 13.

TABLE 13

| | | | Process Differences | |
|---|---|---|---|---|
| | Process D | Process E | Process D/Process E | Results |
| Extraction | Homogenization | Homogenization | N/A | N/A |
| | Cationic Polymer/ | Cationic Polymer/ | N/A | N/A |
| | Dilution | Dilution | | |
| | | Flocculation | (1) Added a flocculation | (1) Enhanced impurity |
| | | Step/Dilution | step, (2) increased | removal and increased |
| | | | dilution by 82% | pool stability (2) |
| | | | | Improved product |
| | | | | recovery |
| | Centrifugation | Centrifugation | (1) Increased feed flow | (1) Improved process |
| | | | rate by 2-fold | efficiency |
| Chrom 1 | Protein A | Protein A | (1) Changed protein A | (1) Increased product |
| | Resin 1 | Resin 2 | Resin 1 to protein A | binding capacity, |
| | | | Resin 2, (2) removed | reduced ECP, color, and |
| | | | EDTA from | leached protein A ligand |
| | | | equilibration, wash1, | (2) reduced |
| | | | wash 3 buffers, (3) | environmental impact, |
| | | | lowered feed, wash 1, | (3) increased product |
| | | | and elution flow rates | binding capacity (4) |
| | | | (4) Changed | improved resin cleaning, |
| | | | regeneration buffer to | (5) reduced ECP and |
| | | | NaOH, (5) changed | pool conductivity |
| | | | elution buffer to glycine | |
| | | | phosphate | |
| Chrom 2 | Weak CE | Weak AE | (1) Changed from weak | (1) Increased ECP and |
| | | | CE to weak AE; | product variant removal; |
| | | | changed from bind & | improved process |
| | | | elute to flow-through | robustness and |
| | | | | efficiency |
| Chrom 3 | Strong CE | Strong CE | (1) Reduced gradient | (1) Improved process |
| | | | volume by 6 CV's, (2) | efficiency, (2) improved |
| | | | Decreased max resin | process robustness |
| | | | load density by 23% | |
| Final | Strong AE | Strong AE | (1) Decreased max resin | (1) Improved process |
| Chrom | | | load density by 33%, (2) | robustness, (2) |
| | | | Increased adjusted pool | improved facility fit |
| | | | pH from 6.0 to 7.3 | |
| Buffer | UFDF | UFDF | N/A | N/A |
| Exchange | | | | |

A flocculation step was added to Process D. Holding the centrate at elevated temperatures as shown in Table 14 for prolonged periods as in Process E resulted in flocculation of some impurities that otherwise eluted in the protein A pool. However, the flocculation step results in increased turbidity which impedes the protein A loading processes. By testing multiple temperatures and times used to induce the flocculation step upstream, any added turbidity could be minimized and/or removed using the existing centrifugation and filtration techniques in the process without compromising the enhanced purification.

TABLE 14

| | | Centrate Turbidity | | | | E. coli Protein (ECP) (ng/mL) | |
|---|---|---|---|---|---|---|---|
| | | | Load Start | Load End | | | Protein A |
| Temp | Time at | Centrate (no | on Protein | on Protein | | | Resin 1 |
| (° C.) | Temp (hr) | filtration) | A Resin 2 | A Resin 2 | Pool | Centrate | MSS Pool |
| 5 | 5 | Clear | Clear | Turbid | Clear | 7,270,000 | 11,300 |
| 5 | 26 | Clear | Clear | Turbid | | 6,750,000 | 11,000 |
| 15 | 0.5 | Clear | Clear | Turbid | | 6,420,000 | 11,300 |
| | | | | | | | (756 ppm) |
| 15 | 24 | Turbid | Clear | Clear | | 6,260,000 | 8,800 |
| | | | | | | | (643 ppm) |
| 30 | 4 | Very | Clear | Clear | | 6,360,000 | 9,300 |
| | | turbid | | | | | (654 ppm) |
| 30 | 27 | Very | Clear | Clear | | 8,110,000 | 6,700 |
| | | turbid | | | | | (497 ppm) |

In addition, the protein A resin was changed between Process D and Process E after screening different protein A resins. A comparison of protein A resins as shown in Table 15 shows that protein A Resin 2 (MabSelect Sure™) resulted in significantly lower ECP's compared to protein A Resin 1 and Prosep Ulta Plus (PUP). Additionally, protein A Resin 2 cleared PEI to below detectable levels, while protein A Resin 1 and PUP did not. Residual PEI can be problematic because residual PEI can out-compete product for binding domains on the downstream resins, thereby reducing product binding capacity and resulting in erratic behavior. The presence of even small concentrations of residual PEI can be detrimental to the purification efficiency. In the Process D, which uses protein A Resin 1 as the protein A resin, the product must first be processed over the weak CE step to achieve levels of PEI comparable to protein A Resin 2. The ability of protein A Resin 2 to clear residual cationic polymer flocculant (PEI) from the protein A resin load comprising onartuzumab was unique and unexpected. The efficacy of protein A Resin 2 is valuable because of the enhanced flexibility and process robustness it affords. Further, protein A Resin 2 did not leach protein A ligand (results <2 ng/mg) compared to protein A Resin 1 which averages 21 ng/mg, and protein A Resin 2 pools have reduced color compared with Prosep vA and PUP (data not shown).

TABLE 15

| | Average ECP | Polyethyleneimine |
|---|---|---|
| Sample | (ng/mg) | (PEI) (μg/mL) |
| Protein A Resin 1 Pool #1 | 1400 | 87 |
| Protein A Resin 1 Pool #2 | 1400 | 128 |
| PUP Pool | 1200 | 54 |
| Protein A Resin 2 Pool #1 | 960 | <30 |
| Protein A Resin 2 Pool #2 | 910 | N/A |
| Prosep vA Pool¹ | N/A | 500 |
| Weak CE Pool¹ | N/A | <30 |

¹For comparison, the results from a separate experiment are shown where the protein A resin 1 pool is subsequently processed over the weak CE column.

Further, a comparison between protein A elution buffers showed that glycine/phosphoric acid resulted in adjusted pools with lower conductivity (after adjustment to high pH for loading the downstream weak AE resin) and comparable pool volume, pool pH, titrant volume and yield to acetate/acetic acid elution buffers as shown in Table 16. The reduction in adjusted pool conductivity realized with the glycine/phosphoric acid elution buffer represented a significant improvement manufacturing efficiency as the pool did not require a 1:1 dilution, resulting in a 50% reduction in load volume/load process time compared to Process D.

TABLE 16

| | | | | | | | 150 mM glycine/ | | |
|---|---|---|---|---|---|---|---|---|---|
| | | | | | | | 38 mM | 15 mM | 6.5 mM |
| | | | | | | 50 mM | phosphoric | phosphoric | phosphoric |
| | Composition | 100 mM acetate | | | | acetate | acid | acid | acid |
| Elution | pH | 2.9 | 3.3 | 3.7 | 4.1 | 3.3 | 3.3 | 3.7 | 4.1 |
| Buffer | | (n = 2) | | | | | | | |
| | Conductivity | | 0.55 | 0.86 | 1.68 | 0.39 | 1.96 | 0.85 | 0.38 |
| | (mS/cm) | | | | | | | | |
| Protein A | Pool Volume | 1X, 1X | 1X | 1.2X | 6.3X | 1X | 1.2X | 1.5X | 5.2X |
| Resin 2 | (normalized) | | | | | | | | |
| Pool | Adjusted Pool | 5.2, 4.3 | 4.9 | 4.8 | 4.7 | 4.2 | 3.1 | 2.0 | 0.7 |
| | Conductivity | | | | | | | | |
| | (mS/cm) | | | | | | | | |
| | Yield (%) | 113, 110 | 110 | 110 | 85 | 109 | 110 | 110 | 87 |

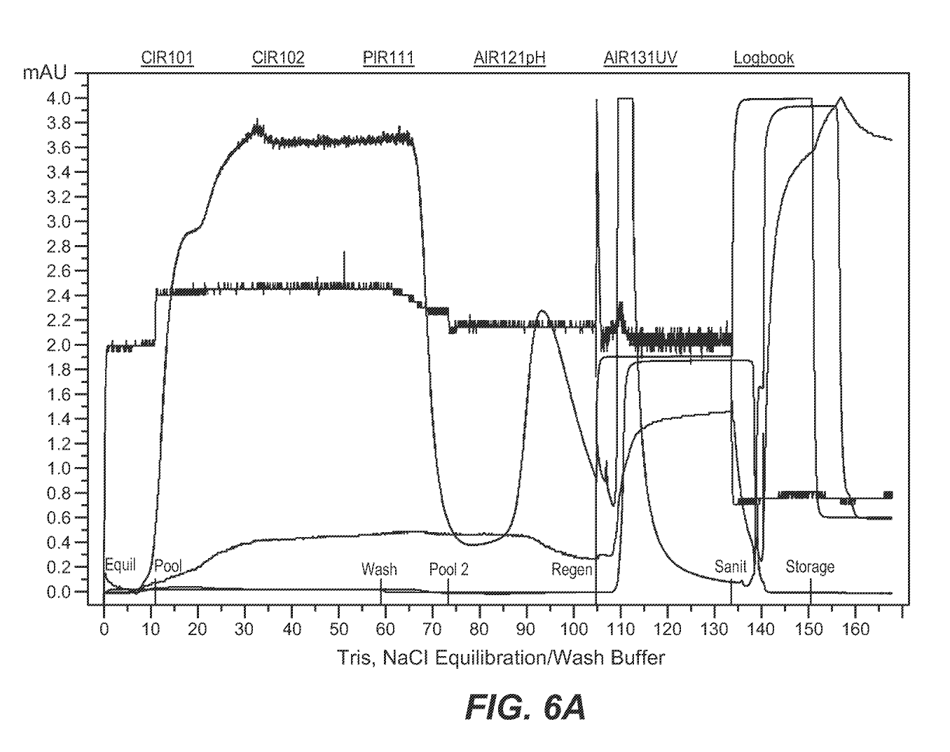

The second chromatography step (Chrom 2) was also changed between Process D and Process E. A high throughput robot screen of 28 resins was conducted in an effort to identify a more effective alternative to the weak CE resin (Chrom 2 step). The weak CE resin was the least effective step at removing ECP and was previously largely necessitated due to its ability to handle residual PEI. With residual PEI no longer an issue due to protein A Resin 2, a more effective Chrom 2 resin was desired. Initially, 12 AE resins, 8 CE resins, and 8 HIC resins were screened for product binding. From this screen, 8 AE resins, 8 CE resins, and 4 HIC resins were further tested for ECP binding using protein A Resin 2 pool as load. For each resin, 48 conditions were tested resulting in the collection of over 2300 data points. Surprisingly, for virtually all tested resins, there was a strong correlation between product and ECP adsorption. These observations, coupled with the results of other analysis performed on final chromatography (Final Chrom) pool (data not shown), suggest the problematic ECP's (i.e. those retained throughout the process) share similar electrostatic and hydrophobic properties to the product, thus making for an exceptionally challenging separation. From the robot screen, the only resin type to show a discernable difference between the onartuzumab product and ECP were weak AE resins, and even here the operating window was small (see graph above for Capto DEAE and blue box for operating window) as shown in FIG. 5.

In comparing Process E and Process F, the differences resulted in a significant improvement in purification process and/or purity of the composition comprising onartuzumab observed as outlined in Table 17.

TABLE 17

| | | | Process Differences | |
|---|---|---|---|---|
| | Process E | Process F | Process E/Process F | Results |
| Extraction | Homogenization | Homogenization | N/A | N/A |
| | Cationic Polymer/ | Cationic Polymer/ | N/A | N/A |
| | Dilution | Dilution | | |
| | Flocculation | Flocculation | N/A | N/A |
| | Step/Dilution | Step/Dilution | | |
| | Centrifugation | Centrifugation | N/A | N/A |
| Chrom 1 | Protein A | Protein A | (1) Increased max load | (1) Improved |
| | Resin 2 | Resin 2 | density by 10% | facility fit |
| Chrom 2 | Weak AE | Weak AE | (1) Changed equilibration | (1) Improved yield |
| | | | buffer from Tris NaCl to | and process |
| | | | glycine phosphate Tris; | robustness and |
| | | | changed end pooling | efficiency, (2) |
| | | | criteria, (2) increased max | Improved facility |
| | | | load density by 10% | fit |
| Chrom 3 | Strong CE | Strong CE | (1) Reduced load | (1) improved |
| | | | conductivity | process robustness |
| Final | Strong AE | Strong AE | (1) Reduced load | (1) Improved |
| Chrom | | | conductivity, (2) Increased | process robustness, |
| | | | max load density by 15% | (2) improved |
| | | | | facility fit |
| Buffer | UFDF | UFDF | N/A | N/A |
| Exchange | | | | |

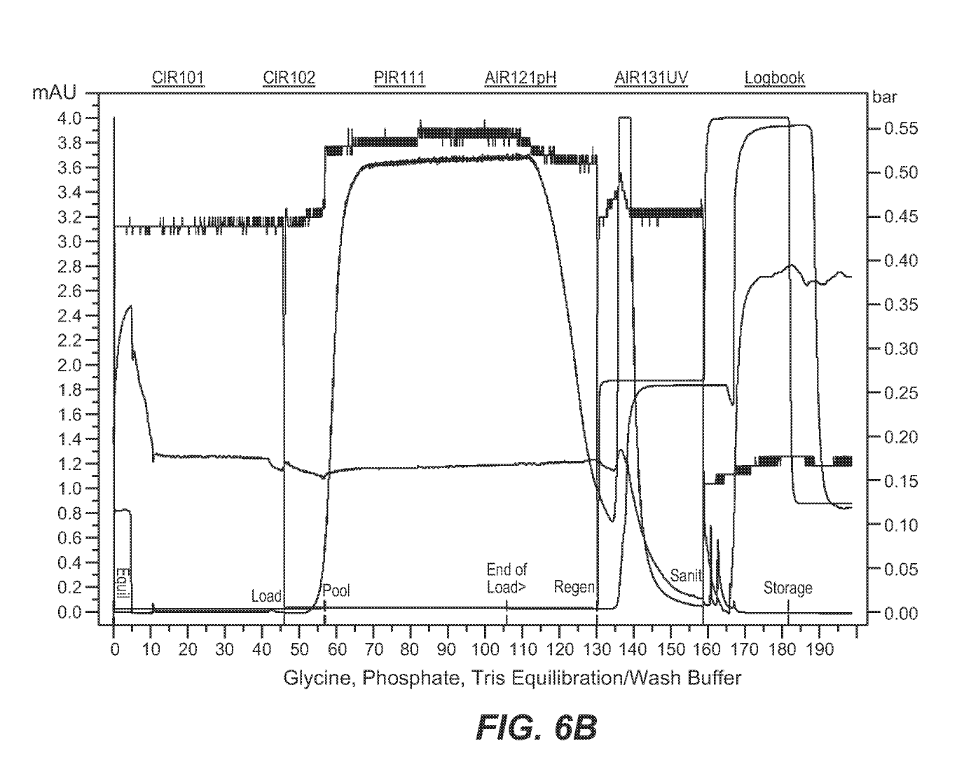

A comparison between weak AE equilibration/wash buffers showed that glycine, phosphate, Tris (GPT) buffer resulted in a more box-like, flow-through step by eliminating the inflection on the leading edge and separated wash peak on the backside of the chromatogram. GPT was a more effective buffer in Process F and the benefits of using it included a 25% reduction in pool and buffer volume, reduced variability in chromatogram shape due to small fluctuations in load pH, and robust end-pooling based on optical density instead of volume as shown in FIG. 6.

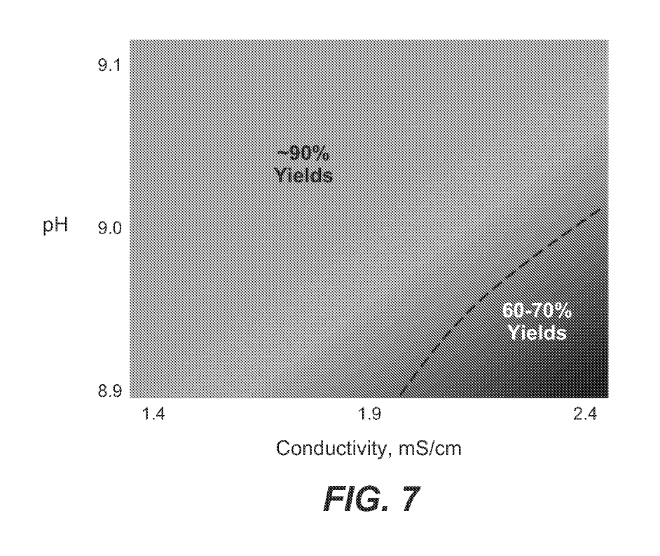

A fractional factorial multi-variate DOE performed on the strong AE final chromatography step revealed an unfavorable interaction between load conductivity and load pH in the lower right-hand corner of the allowable range as shown in FIG. 7. Operating in the vicinity of this corner showed significantly lower yields (60-70%) compared to the other conditions (˜90%). In the vicinity of this corner, and consistent with the loss in yield, a significant breakthrough of the onartuzumab protein was observed in the absorbance signal on the chromatogram (data not shown) toward the end of the load phase, suggesting a reduction in binding capacity due to insufficient charge-charge interactions between the product and resin. To mitigate the risk of premature breakthrough and subsequent yields loss, the target operating conditions for conductivity were left-shifted to avoid the vicinity of the corner in Process F.

### PARTIAL REFERENCE LIST

Angeloni, D. et al. (2003). J Biol Chem. 279(5):3726-3732.
Antipenko, A. et al. (2003). Neuron 39, 589-598.
Bardelli, A. et al. (1997). Oncogene 15, 3103-3111.
Bertotti, A., and Comoglio, P. M. (2003). Trends Biochem Sci 28, 527-533.
Bladt, F. et al. (1995). Nature 376, 768-771.
Blechman, J. M. et al. (1995). Cell 80, 103-113.
Boix, L. et al. (1994). Hepatology 19, 88-91.
Bottaro, D. P. et al. (1991). Science 251, 802-804.
Bussolino, F. et al. (1992). J Cell Biol 119, 629-641.
Coltella, N. et al. (2003). Faseb J 17, 1162-1164.
Cooper, C. S. et al. (1984). Nature 311, 29-33.
Di Renzo, M. F. et al. (1995). Clin Cancer Res 1, 147-154.
Ferguson, K. M. et al. (2003). Mol Cell 11, 507-517.
Furge, K. A. et al. (2000). Oncogene 19, 5582-5589.
Garrett, T. P. et al. (2002). Cell 110, 763-773.
Gherardi, E. et al. (2003). Proc Natl Acad Sci USA.
Giancotti, F. G., and Ruoslahti, E. (1999). Science 285, 1028-1032.
Giordano, S. et al. (2002). Nat Cell Biol 4, 720-724.
Giordano, S. et al. (1989). Oncogene 4, 1383-1388.
Giordano, S. et al. (2000). Faseb J 14, 399-406.
Hamanoue, M. et al. (1996). J Neurosci Res 43, 554-564.
Hartmann, G. et al. (1994). J Biol Chem 269, 21936-21939.
Jeffers, M. et al. (1996). Mol Cell Biol 16, 1115-1125.
Jeffers, M., Schmidt et al. (1997). Proc Natl Acad Sci USA 94, 11445-11450.
Jin, L. et al. (1997). Cancer 79, 749-760.
Kuniyasu, H. et al. (1993). Int J Cancer 55, 72-75.
Lev, S., et al. (1992). J Biol Chem 267, 10866-10873.
Liu, C. et al. (1992). Oncogene 7, 181-185.
Lokker, N. A. et al. (1992). Embo J 11, 2503-2510.
Lorenzato, A. et al. (2002). Cancer Res 62, 7025-7030.
Love, C. A. et al. (2003). Nat Struct Biol 10, 843-848.
Maina, F. et al. (1996). Cell 87, 531-542.
Matsumoto, K., and Nakamura, T. (1993). Exs 65, 225-249.
Maulik, G. et al. (2002). Cytokine Growth Factor Rev 13, 41-59.
Meiners, S. et al. (1998). Oncogene 16, 9-20.
Morello, S. et al. (2001). J Cell Physiol 189, 285-290.
Naka, D. et al. (1992). J Biol Chem 267, 20114-20119.
Naldini, L. et al. (1991). Embo J 10, 2867-2878.
Natali, P. G. et al. (1996). Int J Cancer 69, 212-217.
Nguyen, L. et al. (1997). J Biol Chem 272, 20811-20819.
Nusrat, A. et al. (1994). J Clin Invest 93, 2056-2065.
Ogiso, H. et al. (2002). Cell 110, 775-787.
Olivero, M. et al. (1996). Br J Cancer 74, 1862-1868.
Olivero, M. et al. (1999). Int J Cancer 82, 640-643.
Orian-Rousseau, V. et al. (2002). Genes Dev 16, 3074-3086.
Park, M. et al. (1986). Cell 45, 895-904.
Peek, M. et al. (2002). J Biol Chem 277, 47804-47809.
Pelicci, G. et al. (1995). Oncogene 10, 1631-1638.
Plotnikov, A. N. et al. (1999). Cell 98, 641-650.
Ponzetto, C. et al. (1994). Cell 77, 261-271.
Ponzetto, C. et al. (1996). J Biol Chem 271, 14119-14123.
Robertson, S. C. et al. (2000). Trends Genet 16, 265-271.
Royal, I., and Park, M. (1995). J Biol Chem 270, 27780-27787.
Schmidt, C. et al. (1995). Nature 373, 699-702.
Schmidt, L. et al. (1997). Nat Genet 16, 68-73.
Schmidt, L. et al. (1999). Oncogene 18, 2343-2350.
Suzuki, K. et al. (1994). Hepatology 20, 1231-1236.
Tamagnone, L. et al. (1999). Cell 99, 71-80.
Tempest, P. R. et al. (1988). Br J Cancer 58, 3-7.
Trusolino, L. et al. (2001). Cell 107, 643-654.
Uehara, Y. et al. (1995). Nature 373, 702-705.
Van Vactor, D. V., and Lorenz, L. J. (1999). Curr Biol 9, R201-204.
Weidner, K. M. et al. (1996). Nature 384, 173-176.
Wiesmann, C. et al. (1997). Cell 91, 695-704.
Wiesmann, C. et al. (1999). Nature 401, 184-188.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A composition comprising an anti-c-met antibody, wherein host cell protein (HCP) is present in less than or equal to about 50 ng/mg, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

2. A composition comprising an anti-c-met antibody, wherein HCP is present in less than or equal to about 50 ng/mg, the DNA levels in the composition comprising an anti-c-met antibody are less than or equal to about 0.3 pg/mg, the LpA in the composition comprising an anti-c-met antibody is less than or equal to about 2 ng/mg, the Limulus Amebocyte Lysate (LAL) in the composition comprising an anti-c-met antibody is less than or equal to about 0.01 EU/mg, the percentage of aggregates in the composition comprising an anti-c-met antibody is less than or equal to about 0.3%, the percentage of monomer in the composition comprising an anti-c-met antibody is greater than or equal to about 99.5%, the percentage of fragments in the composition comprising an anti-c-met antibody is less than or equal to about 0.3%, the percentage of acidic variants in the composition comprising an anti-c-met antibody is less than or equal to about 20%, the percentage of main peak in the composition comprising an anti-c-met antibody is greater than or equal to about 75%, and the percentage of basic variants in the composition comprising an anti-c-met antibody is less than or equal to about 2.0%, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

3. A composition comprising an anti-c-met antibody, wherein HCP is present in less than or equal to about 15 ng/mg, the DNA levels in the composition comprising an anti-c-met antibody are less than or equal to about 0.3 pg/mg, the LpA in the composition comprising an anti-c-met antibody is less than or equal to about 2 ng/mg, the Limulus Amebocyte Lysate (LAL) in the composition comprising an anti-c-met antibody is less than or equal to about 0.01 EU/mg, the percentage of aggregates in the composition comprising an anti-c-met antibody is less than or equal to about 0.3%, the percentage of monomer in the composition comprising an anti-c-met antibody is greater than or equal to about 99.5%, the percentage of fragments in the composition comprising an anti-c-met antibody is less than or equal to about 0.3%, the percentage of acidic variants in the composition comprising an anti-c-met antibody is less than or equal to about 20%, the percentage of main peak in the composition comprising an anti-c-met antibody is greater than or equal to about 75%, and the percentage of basic variants in the composition comprising an anti-c-met antibody is less than or equal to about 2.0%, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

4. A method of purifying an anti-c-met antibody comprising keeping a composition comprising the anti-c-met antibody at a temperature of greater than 28° C. and a pH between about pH 6 and about pH 8 for more than 6 hours, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

5. The method of claim 4, wherein the method further comprises centrifuging the composition comprising the anti-c-met antibody.

6. The method of any one of claims 4-5, wherein the method further comprises loading the composition comprising the anti-c-met antibody on MabSelect SuRe resin and eluting the anti-c-met antibody.

7. A method of purifying an anti-c-met antibody comprising loading a composition comprising an anti-c-met antibody on MabSelect SuRe resin and eluting the anti-c-met antibody, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

8. The method of any one of claims 4-7, wherein the method further comprises loading the composition comprising the anti-c-met antibody on a weak anion exchange resin and recovering the anti-c-met antibody in the flow-through.

9. The method of claim 8, wherein the weak anion exchange resin is run in flow-through mode.

10. A method of purifying an anti-c-met antibody comprising loading a composition comprising an anti-c-met antibody on a weak anion exchange resin and recovering the anti-c-met antibody in the flow-through, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

11. The method of claim 10, wherein the weak anion exchange resin is run in flow-through mode.

12. The method of any one of claims 4-11, wherein the method further comprises loading the composition comprising the anti-c-met antibody on a strong cation exchange resin and eluting the anti-c-met antibody.

13. The method of any one of claims 4-12, wherein the method further comprises loading the composition comprising the anti-c-met antibody on a strong anion exchange resin and eluting the anti-c-met antibody.

14. The method of any one of claims 4-13, wherein the method further comprises ultrafiltering and/or diafiltering the composition comprising the anti-c-met antibody.

15. A composition comprising an anti-c-met antibody purified or obtainable by any of the methods of claims 4-14, wherein the anti-c-met antibody comprises a HVR-L1 comprising sequence KSSQSLLYTSSQKNYLA (SEQ ID NO:1), a HVR-L2 comprising sequence WASTRES (SEQ ID NO:2), a HVR-L3 comprising sequence QQYYAYPWT (SEQ ID NO:3), a HVR-H1 comprising sequence GYTFTSYWLH (SEQ ID NO:4), a HVR-H2 comprising sequence GMIDPSNSDTRFNPNFKD (SEQ ID NO:5), and a HVR-H3 comprising sequence ATYRSYVTPLDY (SEQ ID NO:6), wherein the anti-c-met antibody comprises a single antigen binding arm and comprises a Fc region, wherein the Fc region comprises a first and a second Fc polypeptide, and wherein the first and second Fc polypeptides are present in a complex.

16. The composition of claim 15, wherein host cell protein (HCP) is present in less than or equal to about 50 ng/mg.

17. The composition of claim 1-2 or 16, wherein the HCP is present in between about 1 ng/mg and 15 ng/mg.

18. The composition of any one of claim 1-3 or 16-17, wherein the HCP is E. coli protein (ECP).

19. The composition or method of any one of claims 1-18, wherein the anti-c-met antibody comprises (a) a heavy chain variable domain comprising the sequence: EVQLVESGGGLVQPGGSLRLSCAASGYTFTSYWLHWVRQAPGKGLEWVGMIDPSNSDTRFNPN FKDRFTISADTSKNTAYLQMNSLRAEDTAVYYCATYRSYVTPLDYWGQGTLVTVSS (SEQ ID NO:19) and (b) a light chain variable domain comprising the sequence: DIQMTQSPSSLSASVGDRVTITCKSSQSLLYTSSQKNYLAWYQQKPGKAPKLLIYWASTR ESGVPSRFSGSGSGTDFTLTISSLQPEDFATYYCQQYYAYPWTFGQGTKVEIKR (SEQ ID NO:20).

20. The composition or method of claim 19, wherein the Fc region increases stability of said antibody fragment compared to a Fab molecule comprising said antigen binding arm.

21. The composition or method of any one of claims 1-20, wherein the first Fc polypeptide comprises the Fc sequence depicted in FIG. 1 (SEQ ID NO: 17) and the second Fc polypeptide comprises the Fc sequence depicted in FIG. 2 (SEQ ID NO: 18).

22. The composition or method of any one of claims 1-21, wherein the anti-c-met antibody is onartuzumab.

23. The composition or method of any one of claims 1-22, wherein the anti-c-met antibody binds the same epitope as onartuzumab.

24. The composition or method of any one of claims 1-23, wherein the anti-c-met antibody has a pI of between about 8.0 and about 8.5.

25. A pharmaceutical formulation comprising the composition of any one of claim 1-3 or 15-24.

26. A method of inhibiting c-met activated cell proliferation, said method comprising contacting a cell or tissue with an effective amount of the pharmaceutical formulation of claim 25.

27. A method of modulating a disease associated with dysregulation of the HGF/c-met signaling axis, said method comprising administering to a subject an effective amount of the pharmaceutical formulation of claim 25.

28. A method of treating a subject having a proliferative disorder, said method comprising administering to the subject an effective amount of the pharmaceutical formulation of claim 25.

29. The method of claim 28, wherein the proliferative disorder is cancer.

30. The method of claim 29, wherein the cancer is lung cancer, glioblastoma, pancreatic cancer, sarcoma, renal cell carcinoma, hepatocellular carcinoma, gastric cancer, colorectal cancer, and/or breast cancer.

31. The method of any one of claims 26-30, further comprising administering a second therapeutic agent.

32. An article of manufacture comprising a container with the pharmaceutical formulation of claim 25 contained therein.

33. A method of making the article of manufacture of claim 32.


SEQUENCE LISTING (34 sequences)

SEQ ID NO: 1 - protein, 17 aa, Artificial Sequence
  Description of Artificial Sequence Synthetic peptide
KSSQSLLYTSSQKNYLA

SEQ ID NO: 2 - protein, 7 aa, Artificial Sequence
  Description of Artificial Sequence Synthetic peptide
WASTRES

SEQ ID NO: 3 - protein, 9 aa, Artificial Sequence
  Description of Artificial Sequence Synthetic peptide
QQYYAYPWT

SEQ ID NO: 4 - protein, 10 aa, Artificial Sequence
  Description of Artificial Sequence Synthetic peptide
GYTFTSYWLH

SEQ ID NO: 5 - protein, 18 aa, Artificial Sequence
  Description of Artificial Sequence Synthetic peptide
GMIDPSNSDTRFNPNFKD

SEQ ID NO: 6 - protein, 12 aa, Artificial Sequence
  Description of Artificial Sequence Synthetic peptide
ATYRSYVTPLDY

SEQ ID NO: 7 - protein, 23 aa, Artificial Sequence
  Description of Artificial Sequence Synthetic peptide
DIQMTQSPSSLSASVGDRVTITC

SEQ ID NO: 8 - protein, 15 aa, Artificial Sequence
  Description of Artificial Sequence Synthetic peptide
WYQQKPGKAPKLLIY

SEQ ID NO: 9 - protein, 32 aa, Artificial Sequence
  Description of Artificial Sequence Synthetic polypeptide
GVPSRFSGSGSGTDFTLTISSLQPEDFATYYC

SEQ ID NO: 10 - protein, 11 aa, Artificial Sequence
  Description of Artificial Sequence Synthetic peptide
FGQGTKVEIKR

SEQ ID NO: 11 - protein, 25 aa, Artificial Sequence [sequence omitted]

SEQ ID NO: 12 - protein, 13 aa, Artificial Sequence
  Description of Artificial Sequence Synthetic peptide
WVRQAPGKGLEWV

SEQ ID NO: 13 - protein, 30 aa, Artificial Sequence
  Description of Artificial Sequence Synthetic polypeptide
RFTISADTSKNTAYLQMNSLRAEDTAVYYC

SEQ ID NO: 14 - protein, 11 aa, Artificial Sequence
  Description of Artificial Sequence Synthetic peptide
WGQGTLVTVSS

SEQ ID NO: 15 - protein, 106 aa, Artificial Sequence
  Description of Artificial Sequence Synthetic polypeptide
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 108 aa, Artificial Sequence
  Description of Artificial Sequence Synthetic polypeptide
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 17 - protein, 222 aa, Artificial Sequence
  Description of Artificial Sequence Synthetic polypeptide
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 18 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 119 aa, Artificial Sequence [sequence omitted]

SEQ ID NO: 20 - protein, 114 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKR

SEQ ID NO: 21 - protein, 449 aa, Artificial Sequence
  Description of Artificial Sequence Synthetic polypeptide
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 22 - protein, 220 aa, Artificial Sequence
  Description of Artificial Sequence Synthetic polypeptide
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 17 aa, Artificial Sequence
  Description of Artificial Sequence Synthetic peptide
KSSQSLLYTSSQKNYLA

SEQ ID NO: 24 - protein, 7 aa, Artificial Sequence
  Description of Artificial Sequence Synthetic peptide
WASTRES

SEQ ID NO: 25 - protein, 9 aa, Artificial Sequence
  Description of Artificial Sequence Synthetic peptide
QQYYAYPWT

SEQ ID NO: 26 - protein, 10 aa, Artificial Sequence
  Description of Artificial Sequence Synthetic peptide
GYTFTSYWLH

SEQ ID NO: 27 - protein, 18 aa, Artificial Sequence
  Description of Artificial Sequence Synthetic peptide
GMIDPSNSDTRFNPNFKD

SEQ ID NO: 28 - protein, 11 aa, Artificial Sequence
  Description of Artificial Sequence Synthetic peptide
XYGSYVSPLDY

SEQ ID NO: 29 - protein, 11 aa, Artificial Sequence
  Description of Artificial Sequence Synthetic peptide
TYGSYVSPLDY

SEQ ID NO: 30 - protein, 11 aa, Artificial Sequence
  Description of Artificial Sequence Synthetic peptide
SYGSYVSPLDY

SEQ ID NO: 31 - protein, 5 aa, Artificial Sequence
  Description of Artificial Sequence Synthetic peptide
LDAQT

SEQ ID NO: 32 - protein, 9 aa, Artificial Sequence
  Description of Artificial Sequence Synthetic peptide
LTEKRKKRS

SEQ ID NO: 33 - protein, 8 aa, Artificial Sequence
  Description of Artificial Sequence Synthetic peptide
KPDSAEPM

SEQ ID NO: 34 - protein, 8 aa, Artificial Sequence
  Description of Artificial Sequence Synthetic peptide
NVRCLQHF